(19) United States
(12) Patent Application Publication
(10) Pub. No.: US 2016/0031941 A1
(43) Pub. Date: Feb. 4, 2016

(54) TARGETING PEPTIDES THAT BIND S. MUTANS, CONSTRUCTS COMPRISING SUCH PEPTIDES AND USES THEREOF

(71) Applicant: C3 JIAN, INC., Marina Del Rey, CA (US)

(72) Inventors: Randal H. Eckert, Rancho Palos Verdes, CA (US); Christopher W. Kaplan, Los Angeles, CA (US); Pierre A. Kyme, Los Angeles, CA (US); Brian C. Varnum, Santa Monica, CA (US)

(21) Appl. No.: 14/794,609

(22) Filed: Jul. 8, 2015

Related U.S. Application Data
(60) Provisional application No. 62/023,678, filed on Jul. 11, 2014.

(51) Int. Cl. C07K 7/08; A61K 38/10; G01N 33/569; A61K 41/00; A61K 8/64; A61K 47/48; A61K 38/08

(52) U.S. Cl. CPC C07K 7/08; A61K 47/48246; A61K 38/10; A61K 38/08; A61K 41/0057; A61K 8/64; G01N 33/56944; A61Q 11/00

(57) ABSTRACT

In certain embodiments, novel targeting peptides that specifically/preferentially bind to S. mutans are provided. The targeting peptides can be attached to effectors (e.g., detectable labels, drugs, antimicrobial peptides, etc.) to form chimeric constructs for specifically/preferentially delivering the effector to and/or into the target organism. In certain embodiments the targeting peptides attached, e.g., to antimicrobial peptides can be used to selectively inhibit and/or kill S. mutans and, when used in the oral cavity of a mammal, can be effective to reduce the incidence and/or severity of dental caries and/or the incidence and/or severity of periodontal disease.

### CROSS-REFERENCE TO RELATED APPLICATIONS

This application claims benefit of and priority to U.S. Ser. No. 62/023,678, filed on Jul. 11, 2014, which is incorporated herein by reference in its entirety for all purposes.

### STATEMENT OF GOVERNMENTAL SUPPORT

[Not Applicable]

### BACKGROUND

Antibiotic research at the industrial level was originally focused on the identification of refined variants of already existing drugs. This resulted in the development of antibiotics such as newer penicillins, cephalosporins, macrolides, and fluoroquinolones.

However, resistance to old and newer antibiotics among bacterial pathogens is evolving rapidly, as exemplified by extended beta-lactamase (ESBL) and quinolone resistant gram-negatives, multi-resistant gonococci, methicillin resistant Staphylococcus aureus (MRSA), vancomycin resistant enterococci (VRE), penicillin non-susceptible pneumococci (PNSP) and macrolide resistant pneumococci and streptococci (see, e.g., Panlilo et al. (1992) Infect. Control Hosp. Epidemiol., 13: 582-586; Morris et al. (1995) Ann Intern Med., 123: 250-259, and the like). An overuse, or improper use, of antibiotics is believed to be of great importance for triggering and spread of drug resistant bacteria. Microbes have, in many cases, adapted and are resistant to antibiotics due to constant exposure and improper use of the drugs.

Drug resistant pathogens represent a major economic burden for health-care systems. For example, postoperative and other nosocomial infections will prolong the need for hospital care and increase antibiotic drug expenses. It is estimated that the annual cost of treating drug resistant infections in the United States is approximately $5 billion.

### SUMMARY

In certain embodiments, novel targeting peptides that specifically/preferentially bind to microorganisms (e.g., S. mutans, and the like) are provided. The targeting moieties can be attached to effectors (e.g., detectable labels, drugs, antimicrobial peptides, etc.) to form chimeric constructs for specifically/preferentially delivering the effector to and/or into the target organism. In certain embodiments novel antimicrobial peptides that can be used to inhibit (e.g., kill and/or inhibit growth and/or proliferation) of certain microorganisms (e.g., certain bacteria, yeasts, fungi, molds, viruses, algae, protozoa, and the like) are provided.

Accordingly, in certain embodiments, a chimeric construct (chimeric moiety) is provided comprising one or more targeting peptides described herein attached to one or more effectors (e.g., antimicrobial peptides).

Various embodiments contemplated herein may include, but need not be limited to, one or more of the following:

### Embodiment 1

A targeting peptide that binds to Streptococcus mutans, where said peptide comprises or consists of the amino acid sequence X¹-X²-F-R-X⁵-X⁶-X⁷-R-X⁹-X¹⁰-X¹¹-X¹²-X¹³-X¹⁴-X¹⁵-X¹⁶ (SEQ ID NO:1) or the inverse of said amino acid sequence, wherein: X¹ is a polar amino acid, or A; X² is F, W, Q, A, or an analog thereof; X⁵ is a hydrophobic amino acid; X⁶ is a hydrophobic amino acid, N, Q, or an analog thereof; X⁷ is a polar amino acid, A, F, or an analog thereof; X⁹ is a polar amino acid, A or an analog thereof; X¹⁰ is a hydrophobic amino acid, Q, A, or an analog thereof; X¹¹ is a hydrophobic amino acid; X¹² is Q, A, or an analog thereof; X¹³ is a non-polar amino acid; X¹⁴ is a hydrophobic amino acid; X¹⁵ is a non-polar amino acid, N, S, D, or an analog thereof; X¹⁶ is a polar amino acid, F, A, or an analog thereof; and said peptide ranges in length up to 100 amino acids.

### Embodiment 2

The peptide of embodiment 1, wherein X¹ is A or T.

### Embodiment 3

The peptide according to any one of embodiments 1-2, wherein X² is F, W, Q, or A.

### Embodiment 4

The peptide of embodiment 3, wherein X² is F.

### Embodiment 5

The peptide according to any one of embodiments 1-4, wherein X⁵ is L, A, or an analogue thereof.

### Embodiment 6

The peptide of embodiment 5, wherein X⁵ is L or A.

### Embodiment 7

The peptide of embodiment 5, wherein X⁵ is L.

### Embodiment 8

The peptide according to any one of embodiments 1-7, wherein X⁶ is F, L, N, A, Q, or an analog thereof.

### Embodiment 9

The peptide of embodiment 8, wherein X⁶ is F, L, N, A, or Q.

### Embodiment 10

The peptide of embodiment 8, wherein X⁶ is a hydrophobic amino acid.

### Embodiment 11

The peptide of embodiment 8, wherein X⁶ is F.

### Embodiment 12

The peptide according to any one of embodiments 1-11, wherein X⁷ is a polar amino acid, A, or F.

### Embodiment 13

The peptide of embodiment 12, wherein X⁷ is a polar amino acid or A.

### Embodiment 14

The peptide of embodiment 12, wherein X⁷ is a N, A, S, D, or F.

### Embodiment 15

The peptide of embodiment 12, wherein X⁷ is N or A.

### Embodiment 16

The peptide of embodiment 12, wherein X⁷ is N.

### Embodiment 17

The peptide according to any one of embodiments 1-16, wherein X⁹ is a polar amino acid, or A.

### Embodiment 18

The peptide of embodiment 17, wherein X⁹ is S or A.

### Embodiment 19

The peptide of embodiment 17, wherein X⁹ is S.

### Embodiment 20

The peptide according to any one of embodiments 1-19, wherein X¹⁰ is a hydrophobic amino acid, Q, or A.

### Embodiment 21

The peptide of embodiment 20, wherein X¹⁰ is a hydrophobic amino acid.

### Embodiment 22

The peptide of embodiment 21, wherein X¹⁰ is F, L, or an analog thereof.

### Embodiment 23

The peptide of embodiment 21, wherein X¹⁰ is F, or L.

### Embodiment 24

The peptide of embodiment 21, wherein X¹⁰ is F.

### Embodiment 25

The peptide according to any one of embodiments 1-24, wherein X″ is T, A, or an analog thereof.

### Embodiment 26

The peptide of embodiment 25, wherein X″ is T or A.

### Embodiment 27

The peptide of embodiment 25, wherein X″ is T.

### Embodiment 28

The peptide according to any one of embodiments 1-27, wherein X¹² is Q or A.

### Embodiment 29

The peptide of embodiment 28, wherein X¹² is Q.

### Embodiment 30

The peptide according to any one of embodiments 1-29, wherein X¹³ is P, A, or an analog thereof.

### Embodiment 31

The peptide of embodiment 30, wherein X¹³ is P or A.

### Embodiment 32

The peptide of embodiment 30, wherein X¹³ is A.

### Embodiment 33

The peptide according to any one of embodiments 1-32, wherein X¹⁴ is L, A, or an analog thereof.

### Embodiment 34

The peptide of embodiment 33, wherein X¹⁴ is L or A.

### Embodiment 35

The peptide of embodiment 33, wherein X¹⁴ is L.

### Embodiment 36

The peptide according to any one of embodiments 1-35, wherein X¹⁵ is a non-polar amino acid, N, S, or D.

### Embodiment 37

The peptide of embodiment 36, wherein X¹⁵ is G, A, F, N, S, D, or an analog thereof.

### Embodiment 38

The peptide of embodiment 37, wherein X¹⁵ is G, A, F, N, S, or D.

### Embodiment 39

The peptide of embodiment 37, wherein X¹⁵ is G, or A.

### Embodiment 40

The peptide according to any one of embodiments 1-39, wherein X¹⁶ is X¹⁶ is a polar amino acid, F, or A.

### Embodiment 41

The peptide of embodiment 40, wherein X¹⁶ is a polar amino acid.

### Embodiment 42

The peptide of embodiment 41, wherein X¹⁶ is K, Q, or an analog thereof.

### Embodiment 43

The peptide of embodiment 41, wherein X¹⁶ is K or Q.

### Embodiment 44

The peptide of embodiment 41, wherein X¹⁶ is K.

### Embodiment 45

The peptide according to any one of embodiments 1-44, wherein said peptide does not comprise the amino acid sequence TFFRLFNRSFTQALGK.

### Embodiment 46

The peptide of embodiment 1, wherein said peptide comprises or consists of an amino acid sequence selected from the group consisting of AFFRAFNRAFAQALAK (SEQ ID NO:5), TFFRAFARAFAQAAAK (SEQ ID NO:6), AFFRAFARAFAQALAK (SEQ ID NO:7), AFFRLFARAFAQAAAK (SEQ ID NO:8), TLFRLLNRSLTQALGK (SEQ ID NO:9), TFFRLFNRSFTQALFK (SEQ ID NO:10), TFFRLFNRSLTQALGK (SEQ ID NO:11), TFFRLFNRSFTQALNK (SEQ ID NO:12), AFFRAFARAFAQAAAK (SEQ ID NO:13), AFFRAFNRAFAQAAAK (SEQ ID NO:14), TFFRLFNRSFTQALSK (SEQ ID NO:15), AFFRAFARSFAQAAAK (SEQ ID NO:16), AFFRAFARAFAQAAGK (SEQ ID NO:17), AFFRAFARAFTQAAAK (SEQ ID NO:18), TFFRLFNRSFTQALGQ (SEQ ID NO:19), TFFRLLNRSFTQALGK (SEQ ID NO:20), TWFRLFNRSFTQALGK (SEQ ID NO:21), AFFRAFARAFAQAFAK (SEQ ID NO:22), TQFRLFNRSFTQALGK (SEQ ID NO:23), TFFRLFNRSFTQALDK (SEQ ID NO:24), TFFRLFNRSFTQALAK (SEQ ID NO:25), TFFRLFNRSFTQALGE (SEQ ID NO:26), TFFRLFSRSFTQALGK (SEQ ID NO:27), TFFRLFNRSFTQALGA (SEQ ID NO:28), TFFRLFDRSFTQALGK (SEQ ID NO:29), TFFRLFNRSFTQALGF (SEQ ID NO:30), TFFRAFARSFTQAAAK (SEQ ID NO:31), TFFRLFARSFTQAAGK (SEQ ID NO:32), TFFRLFNRSFTQ L K (SEQ ID NO:33), TFFRLFNRSFTQALGS (SEQ ID NO:34), TLFRLFNRSFTQALGK (SEQ ID NO:35), TFFRLNFRSFTQALGK (SEQ ID NO:36), TFFRLFNRSQTQALGK (SEQ ID NO:37), TFFRLFAAAFTQALGK (SEQ ID NO:38), TFFRLFNRSFTQALGK (SEQ ID NO:39), TFFRLFNRSAAAALGK (SEQ ID NO:40), TFFRLFFRSNTQALGK (SEQ ID NO:41), TFFRLFNRSFTQPLGK (SEQ ID NO:42), TAFRLANRSATQALGK (SEQ ID NO:43), TFFRLFNRSFTQAAAA (SEQ ID NO:44), TFFRLQNRSFTQALGK (SEQ ID NO:45), TFFRLFNRSFTQALPK (SEQ ID NO:46), TYYRLFNRSFTQALGK (SEQ ID NO:47), TFFRLF RSFTQALGK (SEQ ID NO:48), and TQFRLQNRSQTQALGK (SEQ ID NO:49).

### Embodiment 47

The peptide of embodiment 46, wherein said peptide comprises or consists of the amino acid sequence AFFRAFNRAFAQALAK (SEQ ID NO:5).

### Embodiment 48

The peptide of embodiment 116, wherein said peptide comprises or consists of the amino acid sequence TFFRAFARAFAQAAAK (SEQ ID NO:6).

### Embodiment 49

The peptide of embodiment 116, wherein said peptide comprises or consists of the amino acid sequence AFFRAFARAFAQALAK (SEQ ID NO:7).

### Embodiment 50

The peptide of embodiment 116, wherein said peptide comprises or consists of the amino acid sequence AFFRLFARAFAQAAAK (SEQ ID NO:8).

### Embodiment 51

The peptide of embodiment 116, wherein said peptide comprises or consists of the amino acid sequence TLFRLLNRSLTQALGK (SEQ ID NO:9).

### Embodiment 52

The peptide of embodiment 116, wherein said peptide comprises or consists of the amino acid sequence TFFRLFNRSFTQALFK (SEQ ID NO:10).

### Embodiment 53

The peptide of embodiment 116, wherein said peptide comprises or consists of the amino acid sequence TFFRLFNRSLTQALGK (SEQ ID NO:11).

### Embodiment 54

The peptide of embodiment 116, wherein said peptide comprises or consists of the amino acid sequence TFFRLFNRSFTQALNK (SEQ ID NO:12).

### Embodiment 55

The peptide of embodiment 116, wherein said peptide comprises or consists of the amino acid sequence AFFRAFARAFAQAAAK (SEQ ID NO:13).

### Embodiment 56

The peptide of embodiment 116, wherein said peptide comprises or consists of the amino acid sequence AFFRAFNRAFAQAAAK (SEQ ID NO:14).

### Embodiment 57

The peptide of embodiment 116, wherein said peptide comprises or consists of the amino acid sequence TFFRLFNRSFTQALSK (SEQ ID NO:15).

### Embodiment 58

The peptide of embodiment 116, wherein said peptide comprises or consists of the amino acid sequence AFFRAFARSFAQAAAK (SEQ ID NO:16.

### Embodiment 59

The peptide of embodiment 116, wherein said peptide comprises or consists of the amino acid sequence AFFRAFARAFAQAAGK (SEQ ID NO:17).

### Embodiment 60

The peptide of embodiment 116, wherein said peptide comprises or consists of the amino acid sequence AFFRAFARAFTQAAAK (SEQ ID NO:18).

### Embodiment 61

The peptide of embodiment 116, wherein said peptide comprises or consists of the amino acid sequence TFFRLFNRSFTQALGQ (SEQ ID NO:19).

### Embodiment 62

The peptide of embodiment 116, wherein said peptide comprises or consists of the amino acid sequence TFFRLLNRSFTQALGK (SEQ ID NO:20).

### Embodiment 63

The peptide of embodiment 116, wherein said peptide comprises or consists of the amino acid sequence TWFRLFNRSFTQALGK (SEQ ID NO:21).

### Embodiment 64

The peptide of embodiment 116, wherein said peptide comprises or consists of the amino acid sequence AFFRAFARAFAQAFAK (SEQ ID NO:22).

### Embodiment 65

The peptide of embodiment 116, wherein said peptide comprises or consists of the amino acid sequence TQFRLFNRSFTQALGK (SEQ ID NO:23).

### Embodiment 66

The peptide of embodiment 116, wherein said peptide comprises or consists of the amino acid sequence TFFRLFNRSFTQALDK (SEQ ID NO:24).

### Embodiment 67

The peptide of embodiment 116, wherein said peptide comprises or consists of the amino acid sequence TFFRLFNRSFTQALAK (SEQ ID NO:25).

### Embodiment 68

The peptide of embodiment 116, wherein said peptide comprises or consists of the amino acid sequence TFFRLFNRSFTQALGE (SEQ ID NO:26).

### Embodiment 69

The peptide of embodiment 116, wherein said peptide comprises or consists of the amino acid sequence TFFRLFSRSFTQALGK (SEQ ID NO:27).

### Embodiment 70

The peptide of embodiment 116, wherein said peptide comprises or consists of the amino acid sequence TFFRLFNRSFTQALGA (SEQ ID NO:28).

### Embodiment 71

The peptide of embodiment 116, wherein said peptide comprises or consists of the amino acid sequence TFFRLFDRSFTQALGK (SEQ ID NO:29).

### Embodiment 72

The peptide of embodiment 116, wherein said peptide comprises or consists of the amino acid sequence TFFRLFNRSFTQALGF (SEQ ID NO:30).

### Embodiment 73

The peptide of embodiment 116, wherein said peptide comprises or consists of the amino acid sequence TFFRAFARSFTQAAAK (SEQ ID NO:31).

### Embodiment 74

The peptide of embodiment 116, wherein said peptide comprises or consists of the amino acid sequence TFFRLFARSFTQAAGK (SEQ ID NO:32).

### Embodiment 75

The peptide of embodiment 116, wherein said peptide comprises or consists of the amino acid sequence TFFRLFNRSFTQ L K (SEQ ID NO:33).

### Embodiment 76

The peptide of embodiment 116, wherein said peptide comprises or consists of the amino acid sequence TFFRLFNRSFTQALGS (SEQ ID NO:34).

### Embodiment 77

The peptide of embodiment 116, wherein said peptide comprises or consists of the amino acid sequence TLFRLFNRSFTQALGK (SEQ ID NO:35).

### Embodiment 78

The peptide of embodiment 116, wherein said peptide comprises or consists of the amino acid sequence TFFRLNFRSFTQALGK (SEQ ID NO:36).

### Embodiment 79

The peptide of embodiment 116, wherein said peptide comprises or consists of the amino acid sequence TFFRLFNRSQTQALGK (SEQ ID NO:37).

### Embodiment 80

The peptide of embodiment 116, wherein said peptide comprises or consists of the amino acid sequence TFFRLFAAAFTQALGK (SEQ ID NO:38).

### Embodiment 81

The peptide of embodiment 116, wherein said peptide comprises or consists of the amino acid sequence TFFRLFNRSFTQALGK (SEQ ID NO:39).

### Embodiment 82

The peptide of embodiment 116, wherein said peptide comprises or consists of the amino acid sequence TFFRLFNRSAAAALGK (SEQ ID NO:40).

### Embodiment 83

The peptide of embodiment 116, wherein said peptide comprises or consists of the amino acid sequence TFFRLFFRSNTQALGK (SEQ ID NO:41).

### Embodiment 84

The peptide of embodiment 116, wherein said peptide comprises or consists of the amino acid sequence TFFRLFNRSFTQPLGK (SEQ ID NO:42).

### Embodiment 85

The peptide of embodiment 116, wherein said peptide comprises or consists of the amino acid sequence TAFRLANRSATQALGK (SEQ ID NO:43).

### Embodiment 86

The peptide of embodiment 116, wherein said peptide comprises or consists of the amino acid sequence TFFRLFNRSFTQAAAA (SEQ ID NO:44).

### Embodiment 87

The peptide of embodiment 116, wherein said peptide comprises or consists of the amino acid sequence TFFRLQNRSFTQALGK (SEQ ID NO:45).

### Embodiment 88

The peptide of embodiment 116, wherein said peptide comprises or consists of the amino acid sequence TFFRLFNRSFTQALPK (SEQ ID NO:46).

### Embodiment 89

The peptide of embodiment 116, wherein said peptide comprises or consists of the amino acid sequence TYYRLFNRSFTQALGK (SEQ ID NO:47).

### Embodiment 90

The peptide of embodiment 116, wherein said peptide comprises or consists of the amino acid sequence TFFRLF RSFTQALGK (SEQ ID NO:48).

### Embodiment 91

The peptide of embodiment 116, wherein said peptide comprises or consists of the amino acid sequence TQFRLQNRSQTQALGK (SEQ ID NO:49).

### Embodiment 92

The peptide according to any one of embodiments 1-91, wherein the amino acid sequence of said peptide is the inverse of said sequence.

### Embodiment 93

The peptide according to any one of embodiments 1-92, wherein said peptide ranges in length up to 50 amino acids.

### Embodiment 94

The peptide according to any one of embodiments 1-92, wherein said peptide ranges in length up to 25 amino acids.

### Embodiment 95

The peptide according to any one of embodiments 1-92, wherein said peptide ranges in length up to 20 amino acids.

### Embodiment 96

The peptide according to any one of embodiments 1-95, wherein said peptide includes said amino acid sequence.

### Embodiment 97

The peptide according to any one of embodiments 1-92, wherein said peptide consists of said amino acid sequence.

### Embodiment 98

The peptide according to any one of embodiments 1-97, wherein said targeting peptide is an “L” peptide.

### Embodiment 99

The peptide according to any one of embodiments 1-97, wherein said targeting peptide is a “D” peptide.

### Embodiment 100

The peptide according to any one of embodiments 1-98, where said peptide is recombinantly expressed.

### Embodiment 101

The peptide according to any one of embodiments 1-99, where said peptide is chemically synthesized.

### Embodiment 102

The peptide according to any one of embodiments 1-101, where said peptide is purified ex vivo.

### Embodiment 103

The peptide according to any one of embodiments 1-97, wherein said targeting peptide is a beta peptide.

### Embodiment 104

The peptide according to any one of embodiments 1-103, wherein said peptide is attached to an effector moiety selected from the group consisting of a detectable label, a porphyrin or other photosensitizer, an antimicrobial peptide, an antibiotic, a ligand, a lipid or liposome, an agent that physically disrupts the extracellular matrix within a community of microorganisms, and a polymeric particle.

### Embodiment 105

The peptide of embodiment 104, wherein said peptide is attached to an antimicrobial peptide.

### Embodiment 106

The peptide of embodiment 105, wherein said peptide is attached to an antimicrobial peptide including or consisting of an amino acid sequence found in Table 4.

### Embodiment 107

The peptide of embodiment 105, wherein said peptide is attached to an antimicrobial peptide including or consisting of an amino acid sequence selected from the group consisting of G2 KNLRIIRKGIHIIKKY* (SEQ ID NO:2), Novispirin G10 KNLRRIIRKGIHIIKKYG (SEQ ID NO:49), Novispirin T10 KNLRRIIRKTIHIIKKYG (SEQ ID NO:50), Novispirin G7 KNLRRIGRKIIHIIKKYG (SEQ ID NO:51), Novispirin T7 KNLRRITRKIIHIIKKYG (SEQ ID NO:52), Ovispirin KNLRRIIRKIIHIIKKYG (SEQ ID NO:53), PGG GLLRRLRKKIGEIFKKYG (SEQ ID NO:54), Protegrin-1 RGGRLCYCRRRFCVCVGR* (SEQ ID NO:55), K-1 GLGRVIGRLIKQIIWRR (SEQ ID NO:56), K-2 VYRKRKSILKIYAKLKGWH (SEQ ID NO:57), K-7 NYRLVNAIFSKIFKKKFIKF (SEQ ID NO:58), K-8 KILKFLFKKVF (SEQ ID NO:59), K-9 FIRKFLKKWLL (SEQ ID NO:60), K-10 KLFKFLRKHLL (SEQ ID NO:61), K-11 KILKFLFKQVF (SEQ ID NO:62), K-12 KILKKLFKFVF (SEQ ID NO:63), K-13 GILKKLFTKVF (SEQ ID NO:64), K-14 LRKFLHKLF (SEQ ID NO:65), K-15 LRKNLRWLF (SEQ ID NO:66), K-16 FIRKFLQKLHL (SEQ ID NO:67), K-17 FTRKFLKFLHL (SEQ ID NO:68), K-18 KKFKKFKVLKIL (SEQ ID NO:69), K-19 LLKLLKLKKLKF (SEQ ID NO:70), K-20 FLKFLKKFFKKLKY (SEQ ID NO:71), K-21 GWLKMFKKIIGKFGKF (SEQ ID NO:72), K-22 GIFKKFVKILYKVQKL (SEQ ID NO:73), and B-33 FKKFWKWFRRF (SEQ ID NO:107).

### Embodiment 108

The peptide of embodiment 105, wherein said peptide is attached to an antimicrobial peptide including or consisting of an amino acid sequence selected from the group consisting of G2 KNLRIIRKGIHIIKKY* (SEQ ID NO:2), Novispirin G10 KNLRRIIRKGIHIIKKYG (SEQ ID NO:49), Novispirin T10 KNLRRIIRKTIHIIKKYG (SEQ ID NO:50), Novispirin G7 KNLRRIGRKIIHIIKKYG (SEQ ID NO:51), Novispirin T7 KNLRRITRKIIHIIKKYG (SEQ ID NO:52), Ovispirin KNLRRIIRKIIHIIKKYG (SEQ ID NO:53), PGG GLLRRLRKKIGEIFKKYG (SEQ ID NO:54), Protegrin-1 RGGRLCYCRRRFCVCVGR* (SEQ ID NO:55), and B-33 FKKFWKWFRRF (SEQ ID NO:107).

### Embodiment 109

The peptide of embodiment 105, wherein said peptide is attached to an antimicrobial peptide including or consisting of the amino acid sequence KLFKFLRKHLL (SEQ ID NO:226), or FLKFLKKFFKKLK (SEQ ID NO:227).

### Embodiment 110

The peptide of embodiment 105, wherein said peptide is attached to an antimicrobial peptide including or consisting of the amino acid sequence KNLRIIRKGIHIIKKY (SEQ ID NO:2).

### Embodiment 111

The peptide according to any one of embodiments 105-110, wherein said antimicrobial peptide is an “L” peptide.

### Embodiment 112

The peptide according to any one of embodiments 105-110, wherein said antimicrobial peptide is a “D” peptide.

### Embodiment 113

The peptide according to any one of embodiments 105-110, wherein said antimicrobial peptide is a beta peptide.

### Embodiment 114

The peptide according to any of embodiments 1-113, wherein said targeting peptide is chemically conjugated to said effector.

### Embodiment 115

The peptide of embodiment 114, wherein said targeting peptide is chemically conjugated to said effector via a linker.

### Embodiment 116

The peptide of embodiment 115, wherein said targeting peptide is chemically conjugated to said effector via a linker including a polyethylene glycol (PEG).

### Embodiment 117

The peptide of embodiment 115, wherein said targeting peptide is chemically conjugated to said effector via a non-peptide linker found in Table 5.

### Embodiment 118

The peptide according to any of embodiments 1-113, wherein said targeting peptide is linked directly to said effector (i.e., without a linker).

### Embodiment 119

The peptide according to any of embodiments 1-113, wherein said targeting peptide is linked to said effector via a peptide linkage.

### Embodiment 120

The peptide of embodiment 119, wherein said effector includes an antimicrobial peptide and said construct is a fusion protein.

### Embodiment 121

The peptide according to any one of embodiments 119 and 120, wherein said targeting peptide is attached to said effector by a peptide linker including or consisting of an amino acid sequence found in Table 5.

### Embodiment 122

The peptide of embodiment 121, wherein said peptide linker comprises or consists of the amino acid sequence GGG.

### Embodiment 123

The peptide according to any one of embodiments 1-122, wherein said peptide bears no terminal protecting groups.

### Embodiment 124

The peptide according to any one of embodiments 1-122, wherein said targeting peptide or a construct including said targeting peptide attached to said effector moiety bears one or more protecting groups.

### Embodiment 125

The peptide of embodiment 124, wherein said one or more protecting groups are independently selected from the group consisting of acetyl, amide, 3 to 20 carbon alkyl groups, Fmoc, Tboc, 9-fluoreneacetyl group, 1-fluorenecarboxylic group, 9-florenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl—Z), 2-bromobenzyloxycarbonyl (2-Br—Z), Benzyloxymethyl (Bom), t-butoxycarbonyl (Boc), cyclohexyloxy (cHxO), t-butoxymethyl (Bum), t-butoxy (tBuO), t-Butyl (tBu), and Trifluoroacetyl (TFA).

### Embodiment 126

The peptide of embodiment 124, wherein said targeting peptide or said targeting peptide attached to said effector moiety includes a protecting group at a carboxyl and/or amino terminus.

### Embodiment 127

The peptide of embodiment 126, wherein a carboxyl terminus is amidated.

### Embodiment 128

The peptide according to any one of embodiments 1-127, wherein said construct is functionalized with a polymer to increase serum halflife.

### Embodiment 129

The peptide of embodiment 128, wherein said polymer includes polyethylene glycol and/or a cellulose or modified cellulose.

### Embodiment 130

An antimicrobial peptide that ranges in length up to 100 amino acids, where said peptide comprises or consists of an amino acid sequence selected from the group consisting of FIGAIARLLSKIFGKR (SEQ ID NO:228), GIFSKLAGKKIKNLLISG (SEQ ID NO:229), GIFSKLAGKKIKNLLISGLKG (SEQ ID NO:230), GLFSKFVGKGIKNFLIKGVK (SEQ ID NO:231), KAYSTPRCKGLFRALMCWL (SEQ ID NO:232), KIFGAIWPLALGALKNLIK (SEQ ID NO:233), GWGSFFKKAAHVGKHVGKAALTHYL (SEQ ID NO:234), RGLRRLGRKIAHGVKKYG (SEQ ID NO:235), RGLRRLGRKIAHGVKKYGPTVLRIIRIAG (SEQ ID NO:236), KIAHGVKKYGPTVLRIIR (SEQ ID NO:237), LLGDFFRKSKEKIGKEFKRIVQRIKDFLRNLVPRTES (SEQ ID NO:238), FLPLIGRVLSGIL (SEQ ID NO:239), IGKFLKKAKKFGKAFVKILKK (SEQ ID NO:240), GKFLKKAKKFGKAFVKIL (SEQ ID NO:241), WFLKFLKKFFKKLKY (SEQ ID NO:242), RGLRRLGRKIAHGVKKY (SEQ ID NO:243), LLGDFFRKSKEKI (SEQ ID NO:244), and ILRWPWWPWRRK (SEQ ID NO:245).

### Embodiment 131

The antimicrobial peptide of embodiment 130, wherein said peptide comprises or consists of the amino acid sequence FIGAIARLLSKIFGKR (SEQ ID NO:228).

### Embodiment 132

The antimicrobial peptide of embodiment 130, wherein said peptide comprises or consists of the amino acid sequence 133: The antimicrobial peptide of embodiment 130, wherein said peptide comprises or consists of the amino acid sequence 134: The antimicrobial peptide of embodiment 130, wherein said peptide comprises or consists of the amino acid sequence 135: The antimicrobial peptide of embodiment 130, wherein said peptide comprises or consists of the amino acid sequence 136: The antimicrobial peptide of embodiment 130, wherein said peptide comprises or consists of the amino acid sequence 137: The antimicrobial peptide of embodiment 130, wherein said peptide comprises or consists of the amino acid sequence 138: The antimicrobial peptide of embodiment 130, wherein said peptide comprises or consists of the amino acid sequence 139: The antimicrobial peptide of embodiment 130, wherein said peptide comprises or consists of the amino acid sequence 140: The antimicrobial peptide of embodiment 130, wherein said peptide comprises or consists of the amino acid sequence 141: The antimicrobial peptide of embodiment 130, wherein said peptide comprises or consists of the amino acid sequence 142: The antimicrobial peptide of embodiment 130, wherein said peptide comprises or consists of the amino acid sequence 143: The antimicrobial peptide of embodiment 130, wherein said peptide comprises or consists of the amino acid sequence 144: The antimicrobial peptide of embodiment 130, wherein said peptide comprises or consists of the amino acid sequence GKFLKKAKKFGKAFVKIL (SEQ ID NO:241).

### Embodiment 145

The antimicrobial peptide of embodiment 130, wherein said peptide comprises or consists of the amino acid sequence WFLKFLKKFFKKLKY (SEQ ID NO:242).

### Embodiment 146

The antimicrobial peptide of embodiment 130, wherein said peptide comprises or consists of the amino acid sequence RGLRRLGRKIAHGVKKY (SEQ ID NO:243).

### Embodiment 147

The antimicrobial peptide of embodiment 130, wherein said peptide comprises or consists of the amino acid sequence LLGDFFRKSKEKI (SEQ ID NO:244).

### Embodiment 148

The antimicrobial peptide of embodiment 130, wherein said peptide comprises or consists of the amino acid sequence ILRWPWWPWRRK (SEQ ID NO:245).

### Embodiment 149

The antimicrobial peptide according to any one of embodiments 130-148, wherein said peptide ranges in length up to 50 amino acids, or up to 40 amino acids.

### Embodiment 150

The antimicrobial peptide according to any one of embodiments 130-149, wherein said peptide includes said amino acid sequence.

### Embodiment 151

The antimicrobial peptide according to any one of embodiments 130-149, wherein said peptide consists of said amino acid sequence.

### Embodiment 152

The antimicrobial peptide according to any one of embodiments 130-151, wherein said antimicrobial peptide bears one or more protecting groups.

### Embodiment 153

The antimicrobial peptide of embodiment 152, wherein said one or more protecting groups are independently selected from the group consisting of acetyl, amide, 3 to 20 carbon alkyl groups, Fmoc, Tboc, 9-fluoreneacetyl group, 1-fluorenecarboxylic group, 9-florenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl—Z), 2-bromobenzyloxycarbonyl (2-Br—Z), Benzyloxymethyl (Bom), t-butoxycarbonyl (Boc), cyclohexyloxy (cHxO), t-butoxymethyl (Bum), t-butoxy (tBuO), t-Butyl (tBu), and Trifluoroacetyl (TFA).

### Embodiment 154

The antimicrobial peptide of embodiment 152, wherein said antimicrobial peptide includes a protecting group at a carboxyl and/or amino terminus.

### Embodiment 155

The antimicrobial peptide of embodiment 154, wherein the carboxyl terminus of said peptide is amidated.

### Embodiment 156

The antimicrobial peptide according to any one of embodiments 130-155, wherein said peptide is functionalized with a polymer to increase serum halflife.

### Embodiment 157

The antimicrobial peptide of embodiment 156, wherein said polymer includes polyethylene glycol and/or a cellulose or modified cellulose.

### Embodiment 158

The antimicrobial peptide according to any one of embodiments 130-157, wherein said peptide is attached to a targeting peptide according to any one of embodiments 1-103.

### Embodiment 159

A pharmaceutical composition including a peptide according to any of embodiments 1-158 in a pharmaceutically acceptable carrier.

### Embodiment 160

The composition of embodiment 159, wherein said composition is formulated as a unit dosage formulation.

### Embodiment 161

The composition of embodiment 159, wherein said composition is formulated for administration by a modality selected from the group consisting of intraperitoneal administration, topical administration, oral administration, inhalation administration, transdermal administration, subdermal depot administration, and rectal administration.

### Embodiment 162

A method of killing or inhibiting the growth or proliferation of a bacterium, said method including: contacting said bacterium or a biofilm including said bacterium with a composition including a targeting peptide according to any one of embodiments 1-103 attached to an antimicrobial peptide, and/or to an antibiotic, and/or to a porphyrin or other photosensitizer; and/or contacting said bacterium or a biofilm including said bacterium with a composition including an antimicrobial peptide according to any one of embodiments 130-157.

### Embodiment 163

A method of reducing or preventing the formation of dental caries and/or the incidence or severity of periodontal disease in a mammal, said method including: administering to the oral cavity of said mammal a composition including a targeting peptide according to any one of embodiments 1-103 attached to an antimicrobial peptide, and/or to an antibiotic, and/or to a porphyrin or other photosensitizer; and/or administering to the oral cavity of said mammal a composition including an antimicrobial peptide according to any one of embodiments 130-156.

### Embodiment 164

The method according to any one of embodiments 162-163, wherein said bacterium or biofilm is in a human and/or said oral cavity is the oral cavity of a human.

### Embodiment 165

The method of embodiment 164, wherein said bacterium or biofilm is in the oral cavity of a human.

### Embodiment 166

The method according to any one of embodiments 162-165 wherein said contacting or administering to the oral cavity includes contacting the teeth and/or gums with said composition.

### Embodiment 167

The method according to any one of embodiments 162-166, wherein said composition includes an antimicrobial peptide according to any one of embodiments 130-157.

### Embodiment 168

The method according to any one of embodiments 162-166, wherein said composition includes a targeting peptide according to any one of embodiments 1-103 attached to an antimicrobial peptide.

### Embodiment 169

The method of embodiment 168, wherein said targeting peptide is attached to an antimicrobial peptide including or consisting of an amino acid sequence found in Table 4.

### Embodiment 170

The method of embodiment 168, wherein said targeting peptide is attached to an antimicrobial peptide including or consisting of an amino acid sequence selected from the group consisting of G2 KNLRIIRKGIHIIKKY* (SEQ ID NO:2), Novispirin G10 KNLRRIIRKGIHIIKKYG (SEQ ID NO:49), Novispirin T10 KNLRRIIRKTIHIIKKYG (SEQ ID NO:50), Novispirin G7 KNLRRIGRKIIHIIKKYG (SEQ ID NO:51), Novispirin T7 KNLRRITRKIIHIIKKYG (SEQ ID NO:52), Ovispirin KNLRRIIRKIIHIIKKYG (SEQ ID NO:53), PGG GLLRRLRKKIGEIFKKYG (SEQ ID NO:54), Protegrin-1 RGGRLCYCRRRFCVCVGR* (SEQ ID NO:55), K-1 GLGRVIGRLIKQIIWRR (SEQ ID NO:56), K-2 VYRKRKSILKIYAKLKGWH (SEQ ID NO:57), K-7 NYRLVNAIFSKIFKKKFIKF (SEQ ID NO:58), K-8 KILKFLFKKVF (SEQ ID NO:59), K-9 FIRKFLKKWLL (SEQ ID NO:60), K-10 KLFKFLRKHLL (SEQ ID NO:61), K-11 KILKFLFKQVF (SEQ ID NO:62), K-12 KILKKLFKFVF (SEQ ID NO:63), K-13 GILKKLFTKVF (SEQ ID NO:64), K-14 LRKFLHKLF (SEQ ID NO:65), K-15 LRKNLRWLF (SEQ ID NO:66), K-16 FIRKFLQKLHL (SEQ ID NO:67), K-17 FTRKFLKFLHL (SEQ ID NO:68), K-18 KKFKKFKVLKIL (SEQ ID NO:69), K-19 LLKLLKLKKLKF (SEQ ID NO:70), K-20 FLKFLKKFFKKLKY (SEQ ID NO:71), K-21 GWLKMFKKIIGKFGKF (SEQ ID NO:72), K-22 GIFKKFVKILYKVQKL (SEQ ID NO:73), and B-33 FKKFWKWFRRF (SEQ ID NO:107).

### Embodiment 171

The method of embodiment 168, wherein said targeting peptide is attached to an antimicrobial peptide including or consisting of an amino acid sequence selected from the group consisting of G2 KNLRIIRKGIHIIKKY* (SEQ ID NO:2), Novispirin G10 KNLRRIIRKGIHIIKKYG (SEQ ID NO:49), Novispirin T10 KNLRRIIRKTIHIIKKYG (SEQ ID NO:50), Novispirin G7 KNLRRIGRKIIHIIKKYG (SEQ ID NO:51), Novispirin T7 KNLRRITRKIIHIIKKYG (SEQ ID NO:52), Ovispirin KNLRRIIRKIIHIIKKYG (SEQ ID NO:53), PGG GLLRRLRKKIGEIFKKYG (SEQ ID NO:54), Protegrin-1 RGGRLCYCRRRFCVCVGR* (SEQ ID NO:55), and B-33 FKKFWKWFRRF (SEQ ID NO:107).

### Embodiment 172

The method of embodiment 168, wherein said peptide is attached to an antimicrobial peptide including or consisting of the amino acid sequence KLFKFLRKHLL (SEQ ID NO:226), or FLKFLKKFFKKLK (SEQ ID NO:227).

### Embodiment 173

The method of embodiment 168, wherein said peptide is attached to an antimicrobial peptide including or consisting of the amino acid sequence KNLRIIRKGIHIIKKY (SEQ ID NO:2).

### Embodiment 174

The method according to any one of embodiments 162-173, wherein said targeting peptide is attached to said antimicrobial peptide by a peptide linker including or consisting of an amino acid sequence found in Table 5.

### Embodiment 175

The method of embodiment 174, wherein said peptide linker comprises or consists of the amino acid sequence GGG.

### Embodiment 176

The method of embodiment 162, wherein said targeting peptide is attached to an antimicrobial peptide including or consisting of the amino acid sequence KNLRIIRKGIHIIKKY (SEQ ID NO:2).

### Embodiment 177

A method of detecting a bacterium and/or a bacterial film, said method including: contacting said bacterium or bacterial film with a composition including a targeting peptide according to any one of embodiments 1-103 attached to a detectable label; and detecting said detectable label wherein the quantity and/or location of said detectable label is an indicator of the presence of said bacterium and/or bacterial film.

### Embodiment 178

The method of embodiment 177, wherein said detectable label is a label selected from the group consisting of a radioactive label, a radio-opaque label, a fluorescent dye, a fluorescent protein, an enzymatic label, a colorimetric label, and a quantum dot.

### Embodiment 179

A composition including a targeting peptide according to any one of embodiments 1-103 attached to a photosensitizing agent.

### Embodiment 180

The composition of embodiment 179, wherein said photosensitizing agent is an agent selected from the group consisting of a porphyrinic macrocycle, a porphyrin, a chlorine, a crown ether, an acridine, an azine, a phthalocyanine, a cyanine, a cucumin, a psoralen, and a perylenequinonoid.

### Embodiment 181

The composition of embodiment 179, wherein said photosensitizing agent is an agent shown in any of FIGS. 1-12.

### Embodiment 182

The composition of embodiment 179, wherein said photosensitizing agent is attached to said targeting peptide by a non-peptide linker.

### Embodiment 183

The composition of embodiment 179, wherein said photosensitizing agent is attached to said targeting peptide by a linker including a polyethylene glycol (PEG).

### Embodiment 184

The composition of embodiment 179, wherein said photosensitizing agent is attached to said targeting peptide by a non-peptide linker found in Table 5.

### Embodiment 185

A method of inhibiting the growth or proliferation of a microorganism or a biofilm, said method including contacting said microorganism or biofilm with a composition according to any of embodiments 178-184.

### Embodiment 186

A method of reducing or preventing the formation of dental caries and/or the incidence or severity of periodontal disease in a mammal, said method including: administering to the oral cavity of said mammal a composition according to any of embodiments 178-184.

### Embodiment 187

The method according to any one of embodiments 185-186, further including exposing said microorganism and/or biofilm and/or composition to a light source.

### Embodiment 188

The method of embodiment 185, wherein said microorganism is a microorganism selected from the group consisting of a bacterium, a yeast, a fungus, a protozoan, and a virus.

### Embodiment 189

The method of embodiment 185, wherein said biofilm includes a bacterial film.

### Embodiment 190

The method of embodiment 185, wherein said biofilm is a biofilm on an implanted or implantable medical device.

### Embodiment 191

The method of embodiment 185, wherein said microorganism or biofilm is an organism or biofilm in an oral cavity.

### DEFINITIONS

The term “peptide” as used herein refers to a polymer of amino acid residues typically ranging in length from 2 to about 30, or to about 40, or to about 50, or to about 60, or to about 70 residues. In certain embodiments the peptide ranges in length from about 2, 3, 4, 5, 7, 9, 10, or 11 residues to about 60, 50, 45, 40, 45, 30, 25, 20, or 15 residues. In certain embodiments the peptide ranges in length from about 8, 9, 10, 11, or 12 residues to about 15, 20 or 25 residues. In certain embodiments the amino acid residues comprising the peptide are “L-form” amino acid residues, however, it is recognized that in various embodiments, “D” amino acids can be incorporated into the peptide. Peptides also include amino acid polymers in which one or more amino acid residues is an artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an α-ester, a β-ester, a thioamide, a phosphonamide, a carbomate, a hydroxylate, and the like (see, e.g., Spatola, (1983) Chem. Biochem. Amino Acids and Proteins 7: 267-357), where the amide is replaced with a saturated amine (see, e.g., Skiles et al., U.S. Pat. No. 4,496,542, which is incorporated herein by reference, and Kaltenbronn et al., (1990) Pp. 969-970 in Proc. 11th American Peptide Symposium, ESCOM Science Publishers, The Netherlands, and the like)).

The term “residue” as used herein refers to natural, synthetic, or modified amino acids. Various amino acid analogues include, but are not limited to 2-aminoadipic acid, 3-aminoadipic acid, beta-alanine (beta-aminopropionic acid), 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4 diaminobutyric acid, desmosine, 2,2′-diaminopimelic acid, 2,3-diaminopropionic acid, n-ethylglycine, n-ethylasparagine, hydroxylysine, allo-hydroxylysine, 3-hydroxyproline, 4-hydroxyproline, isodesmosine, allo-isoleucine, n-methylglycine, sarcosine, n-methylisoleucine, 6-n-methyllysine, n-methylvaline, norvaline, norleucine, ornithine, and the like. These modified amino acids are illustrative and not intended to be limiting.

“β-peptides” comprise of “β amino acids”, which have their amino group bonded to the β carbon rather than the α-carbon as in the 20 standard biological amino acids. The only commonly naturally occurring β amino acid is β-alanine.

Peptoids, or N-substituted glycines, are a specific subclass of peptidomimetics. They are closely related to their natural peptide counterparts, but differ chemically in that their side chains are appended to nitrogen atoms along the molecule's backbone, rather than to the α-carbons (as they are in natural amino acids).

The terms “conventional” and “natural” as applied to peptides herein refer to peptides, constructed only from the naturally-occurring amino acids: Ala, Cys, Asp, Glu, Glu, Phe, Gly, His, Ile, Lys, Leu, Met, Asn, Pro, Gln, Arg, Ser, Thr, Val, Trp, and Tyr. A compound of the invention “corresponds” to a natural peptide if it elicits a biological activity (e.g., antimicrobial activity) related to the biological activity and/or specificity of the naturally occurring peptide. The elicited activity may be the same as, greater than or less than that of the natural peptide. In general, such a peptoid will have an essentially corresponding monomer sequence, where a natural amino acid is replaced by an N-substituted glycine derivative, if the N-substituted glycine derivative resembles the original amino acid in hydrophilicity, hydrophobicity, polarity, etc. The following are illustrative, but non-limiting N-substituted glycine replacements: N-(1-methylprop-1-yl)glycine substituted for isoleucine (Ile), N-(prop-2-yl)glycine for valine (Val), N-benzylglycine for phenylanlaine (Phe), N-(2-hydroxyethyl)glycine for serine (Ser), and the like. In certain embodiments substitutions need not be “exact”. Thus for example, in certain embodiments N-(2-hydroxyethyl)glycine may substitute for Ser, Thr, Cys, and/or Met; N-(2-methylprop-1-yl)glycine may substitute for Val, Leu, and/or Ile. In certain embodiments N-(2-hydroxyethyl)glycine can be used to substitute for Thr and Ser, despite the structural differences: the side chain in N-(2-hydroxyethyl)glycine is one methylene group longer than that of Ser, and differs from Thr in the site of hydroxy-substitution. In general, one may use an N-hydroxyalkyl-substituted glycine to substitute for any polar amino acid, an N-benzyl- or N-aralkyl-substituted glycine to replace any aromatic amino acid (e.g., Phe, Trp, etc.), an N-alkyl-substituted glycine such as N-butylglycine to replace any nonpolar amino acid (e.g., Leu, Val, Ile, etc.), and an N-(aminoalkyl)glycine derivative to replace any basic polar amino acid (e.g., Lys and Arg).

Where an amino acid sequence is provided herein, L-, D-, or beta amino acid versions of the sequence are also contemplated as well as retro, inversion, and retro-inversion isoforms. In addition, conservative substitutions (e.g., in the binding peptide, and/or antimicrobial peptide, and/or linker peptide) are contemplated. Non-protein backbones, such as PEG, alkane, ethylene bridged, ester backbones, and other backbones are also contemplated. Also fragments ranging in length from about 5, 6, 7, 8, 9, 10, 11, 12, 13, 14, 15, 16, 17, 18, 19, 20, 21, 22, 23, 24, or 25 amino acids up to the full length minus one amino acid of the peptide are contemplated where the fragment retains at least 50%, preferably at least 60% 70% or 80%, more preferably at least 90%, 95%, 98%, 99%, or at least 100% of the activity (e.g., binding specificity and/or avidity, antimicrobial activity, etc.) of the full length peptide are contemplated.

In certain embodiments, conservative substitutions of the amino acids comprising any of the sequences described herein are contemplated. In various embodiments one, two, three, four, or five different residues are substituted. The term “conservative substitution” is used to reflect amino acid substitutions that do not substantially alter the activity (e.g., antimicrobial activity and/or specificity) of the molecule. Typically conservative amino acid substitutions involve substitution one amino acid for another amino acid with similar chemical properties (e.g. charge or hydrophobicity). Certain conservative substitutions include “analog substitutions” where a standard amino acid is replaced by a non-standard (e.g., rare, synthetic, etc.) amino acid differing minimally from the parental residue. Amino acid analogs are considered to be derived synthetically from the standard amino acids without sufficient change to the structure of the parent, are isomers, or are metabolite precursors. Examples of such “analog substitutions” include, but are not limited to, 1) Lys-Orn, 2) Leu-Norleucine, 3) Lys-Lys[TFA], 4) Phe-Phe[Gly], and 5) 6-amino butylglycine-ξ-amino hexylglycine, where Phe[gly] refers to phenylglycine (a Phe derivative with a H rather than CH₃ component in the R group), and Lys[TFA] refers to a Lys where a negatively charged ion (e.g., TFA) is attached to the amine R group. Other conservative substitutions include “functional substitutions” where the general chemistries of the two residues are similar, and can be sufficient to mimic or partially recover the function of the native peptide. Strong functional substitutions include, but are not limited to 1) Gly/Ala, 2) Arg/Lys, 3) Ser/Tyr/Thr, 4) Leu/Ile/Val, 5) Asp/Glu, 6) Gln/Asn, and 7) Phe/Trp/Tyr, while other functional substitutions include, but are not limited to 8) Gly/Ala/Pro, 9) Tyr/His, 10) Arg/Lys/His, 11) Ser/Thr/Cys, 12) Leu/Ile/Val/Met, and 13) Met/Lys (special case under hydrophobic conditions). Various “broad conservative substations” include substitutions where amino acids replace other amino acids from the same biochemical or biophysical grouping. This is similarity at a basic level and stems from efforts to classify the original 20 natural amino acids. Such substitutions include 1) nonpolar side chains: Gly/Ala/Val/Leu/Ile/Met/Pro/Phe/Trp, and/or 2) uncharged polar side chains Ser/Thr/Asn/Gln/Tyr/Cys. In certain embodiments broad-level substitutions can also occur as paired substitutions. For example, Any hydrophilic neutral pair [Ser, Thr, Gln, Asn, Tyr, Cys]+[Ser, Thr, Gln, Asn, Tyr, Cys] can may be replaced by a charge-neutral charged pair [Arg, Lys, His]+[Asp, Glu]. The following six groups each contain amino acids that, in certain embodiments, are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K), Histidine (H); 5) Isoleucine (I), Leucine (L), Methionine (M), Valine (V); and 6) Phenylalanine (F), Tyrosine (Y), Tryptophan (W). Where amino acid sequences are disclosed herein, amino acid sequences comprising, one or more of the above-identified conservative substitutions are also contemplated.

In certain embodiments, targeting peptides, antimicrobial peptides, and/or STAMPs compromising at least 80%, preferably at least 85% or 90%, and more preferably at least 95% or 98% sequence identity with any of the sequences described herein are also contemplated. The terms “identical” or percent “identity,” refer to two or more sequences that are the same or have a specified percentage of amino acid residues that are the same, when compared and aligned for maximum correspondence, as measured using one of the following sequence comparison algorithms or by visual inspection. With respect to the peptides of this invention sequence identity is determined over the full length of the peptide. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman (1981) Adv. Appl. Math. 2: 482, by the homology alignment algorithm of Needleman & Wunsch (1970) J. Mol. Biol. 48: 443, by the search for similarity method of Pearson & Lipman (1988) Proc. Natl. Acad. Sci., USA, 85: 2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

The term “specificity” when used with respect to the antimicrobial activity of a peptide indicates that the peptide preferentially inhibits growth and/or proliferation and/or kills a particular microbial species as compared to other related and/or unrelated microbes. In certain embodiments the preferential inhibition or killing is at least 10% greater (e.g., LD₅₀ is 10% lower), preferably at least 20%, 30%, 40%, or 50%, more preferably at least 2-fold, at least 5-fold, or at least 10-fold greater for the target species.

“Treating” or “treatment” of a condition as used herein may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

The term “consisting essentially of” when used with respect to an antimicrobial peptide (AMP) or AMP motif as described herein, indicates that the peptide or peptides encompassed by the library or variants, analogues, or derivatives thereof possess substantially the same or greater antimicrobial activity and/or specificity as the referenced peptide. In certain embodiments substantially the same or greater antimicrobial activity indicates at least 80%, preferably at least 90%, and more preferably at least 95% of the antimicrobial activity of the referenced peptide(s) against a particular bacterial species (e.g., S. mutans).

The term “STAMP” refers to Specifically Targeted Anti-Microbial Peptides. In various embodiments, a STAMP comprises one or more targeting peptides attached to one or more antimicrobial moieties (e.g., antimicrobial peptides (AMPs)). An MH-STAMP is a STAMP bearing two or more targeting domains (i.e., a multi-headed STAMP).

The terms “isolated” “purified” or “biologically pure” refer to material which is substantially or essentially free from components that normally accompany it as found in its native state. In the case of a peptide, an isolated (naturally occurring) peptide is typically substantially free of components with which it is associated in the cell, tissue, or organism. The term isolated also indicates that the peptide is not present in a phage display, yeast display, or other peptide library.

In various embodiments the amino acid abbreviations shown in Table 1 are used herein.

TABLE 1
Amino acid abbreviations.

Abbreviation

| Name | 3 Letter | 1 Letter |
|---|---|---|
| Alanine | Ala | A |
| βAlanine | βAla | |
| (NH₂—CH₂—CH₂—COOH) | | |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Homoserine | Hse | — |
| Isoleucine | Ile | I |
| Leucine | Leu | L |
| Lysine | Lys | K |
| Methionine | Met | M |
| Methionine sulfoxide | Met (O) | — |
| Methionine methylsulfonium | Met (S—Me) | — |
| Norleucine | Nle | — |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| episilon-aminocaproic acid | Ahx | J |
| (NH²—(CH₂)₅—COOH) | | |
| 4-aminobutanoic acid | gAbu | |
| (NH₂—(CH₂)₃—COOH) | | |
| tetrahydroisoquinoline-3- | | O |
| carboxylic acid | | |
| Lys(N(epsilon)-trifluoroacetyl) | | K[TFA] |
| α-aminoisobutyric acid | Aib | B |

### BRIEF DESCRIPTION OF THE DRAWINGS

FIG. 1 shows some illustrative porphyrins (compounds 92-99) suitable for use as targeting moieties and/or antimicrobial effectors.

FIG. 2 shows some illustrative porphyrins (compounds 100-118) suitable for use as targeting moieties and/or antimicrobial effectors.

FIG. 3 shows some illustrative porphyrins (in particular phthalocyanines) (compounds 119-128) suitable for use as targeting moieties and/or antimicrobial effectors.

FIG. 4 illustrates the structures of two phthalocyanines, Monoastral Fast Blue B and Monoastral Fast Blue G suitable for use as antimicrobial effectors.

FIG. 5 illustrates certain azine photosensitizers suitable for use as antimicrobial effectors in the compositions and methods described herein.

FIG. 6 shows illustrative cyanine suitable for use as antimicrobial effectors in the compositions and methods described herein.

FIG. 7 shows illustrative psoralen (angelicin) photosensitizers suitable for use as antimicrobial effectors in the compositions and methods described herein.

FIG. 8 shows illustrative hypericin and the perylenequinonoid pigments suitable for use as antimicrobial effectors in the compositions and methods described herein.

FIG. 9 shows illustrative acridines suitable for use as antimicrobial effectors in the compositions and methods described herein.

FIG. 10 illustrates the structure of the acridine Rose Bengal.

FIG. 11 illustrates various crown ethers suitable for use as antimicrobial effectors in the compositions and methods described herein.

FIG. 12 illustrates the structure of cumin.

FIG. 13 illustrates an example of a targeted light-activated porphyrin comprising a porphyrin coupled to a targeting peptide (SEQ ID NO:1).

FIG. 14 schematically shows some illustrative configurations for chimeric constructs described herein. A: Shows a single targeting peptide T1 attached to a single effector E1 by a linker/spacer L that, in certain embodiments, may be omitted. B: Shows multiple targeting peptides T1, T2, T3 attached directly to each other and attached by a linker L to a single effector E1. In various embodiments T1, T2, and T3, can be domains in a fusion protein. C: Shows multiple targeting peptides T1, T2, T3 attached to each other by linkers L and attached by a linker L to a single effector E1. In various embodiments T1, T2, and T3, can be domains in a fusion protein. D: Shows a single targeting peptide T1 attached by a linker L to multiple effectors E1, E2, and E3 joined directly to each other. E: Shows a single targeting peptide T1 attached by a linker L to multiple effectors E1, E2, and E3 joined to each other by linkers L. F: Shows multiple targeting peptides joined directly to each other and by a linker L to multiple effectors joined to each other by linkers L. G: Shows multiple targeting peptides joined to each other by linkers L and by a linker L to multiple effectors joined to each other by linkers L. In various embodiments T1, T2, and T3, and/or E1, E2, and E3 can be domains in a fusion protein. H: Illustrates a branched configuration where multiple targeting peptides are linked to a single effector. I: Illustrates a dual branched configuration where multiple targeting peptides are linked to multiple effectors. J: Illustrates a branched configuration where multiple targeting peptides are linked to multiple effectors where the effectors are joined to each other in a linear configuration. In any of these illustrative, but non-limiting embodiments, one or more linkers may be eliminated and the targeting peptide(s) can be linked directly to one or more effector(s).

### DETAILED DESCRIPTION

In various embodiments targeting peptides are provided that bind (e.g., that preferentially and/or specifically bind to a microorganism (e.g., a bacterium, a fungus, a yeast, etc.). One or more such targeting peptides can be attached to one or more “effector moieties” (e.g., a detectable label, a porphyrin or other photosensitizer, an antimicrobial peptide, an antibiotic, a ligand, a lipid or liposome, an agent that physically disrupts the extracellular matrix within a community of microorganisms, and a polymeric particle, etc.) to provide chimeric moieties that are capable of delivering the effector(s) to a target (e.g., a bacterium, a biofilm comprising the bacterium, etc.). IN certain embodiments, the targeting peptides are attached (directly or through a linker) to an antimicrobial peptide (AMP) thereby affording specificity/selectivity to the antimicrobial peptide. Such constructs may be designated as Specifically-Targeted Antimicrobial Peptides or “STAMPs”.

In various embodiments, targeting peptides include, but are not limited to peptides that preferentially bind particular microorganisms (e.g., S. mutans).

Certain preferred targeting peptides that bind, inter alia, Streptocossus mutans comprise or consist of the amino acid sequence
X¹-X²-F-R-X⁵-X⁶-X⁷-R-X⁹-X¹⁰-X¹¹-X¹²-X¹³-X¹⁴-X¹⁵-X¹⁶ (SEQ ID NO:1)
   or the inverse of said amino acid sequence, wherein X¹ is a polar amino acid, or A; X² is F, W, Q, A, or an analog thereof; X⁵ is a hydrophobic amino acid; X⁶ is a hydrophobic amino acid, N, Q, or an analog thereof; X⁷ is a polar amino acid, A, F, or an analog thereof; X⁹ is a polar amino acid, A or an analog thereof; X¹⁰ is a hydrophobic amino acid, Q, A, or an analog thereof; X¹¹ is a hydrophobic amino acid; X¹² is Q, A, or an analog thereof; X¹³ is a non-polar amino acid; X14 is a hydrophobic amino acid; X15 is a non-polar amino acid, N, S, D, or an analog thereof; X16 is a polar amino acid, F, A, or an analog thereof; and said peptide ranges in length up to 100 amino acids. The peptide does not comprise or consist of the amino acid sequence of C16 (TFFRLFNRSFTQALGK (SEQ ID NO:2).

In certain embodiments, X¹ is a polar amino acid or A, and in certain embodiments A or T; and/or X² is F, W, Q, A, and in certain embodiments F; and/or X⁵ is a hydrophobic amino acid in certain embodiments L or A; and in certain embodiments L; and/or X⁶ is a hydrophobic amino acid, N or Q, in certain embodiments F, L, N, A, or Q; in certain embodiments hydrophobic; and in certain embodiments F; and/or X⁷ is a polar amino acid, A, or F; in certain embodiments a polar amino acid or A; in certain embodiments N, A, S, D, or F; in certain embodiments N or A, and in certain embodiments N; and/or X⁹ is a polar amino acid or A, in certain embodiments S or A, and in certain embodiments preferably S; and/or X¹⁰ is a hydrophobic amino acid, Q, or A, in certain embodiments a hydrophobic amino acid, in certain embodiments F or L, and in certain embodiments F; X¹¹ is a hydrophobic amino acid, in certain embodiments T or A, and in certain embodiments T; and/or X¹² is a Q or A, and in certain embodiments Q; and/or X¹³ is a non-polar amino acid, in certain embodiments P or A, and in certain embodiments preferably A; and/or X¹⁴ is a hydrophobic amino acid, in certain embodiments L or A, and in certain embodiments L; and/or X¹⁵ is a non-polar amino acid, N, S, or D, in certain embodiments G, A, F, N, S, or D, and in certain embodiments G or A; and/or X¹⁶ is a polar amino acid, F, or A, in certain embodiments a polar amino acid, in certain embodiments K or Q, and in certain embodiments K.

In certain embodiments the targeting peptide comprises one or more of the amino acid sequences shown in Table 1.

TABLE 2
S. mutans targeting peptides. Anti-biofilm activity level (%
viability remaining for S. mutans) is shown for a construct comprising
the targeting peptide attached to an antimicrobial peptide
(KNLRIIRKGIHIIKKY (SEQ ID NO: 3)) by a GGG linker. It is noted
that the C16G2 construct (TFFRLFNRSFTQALGK GGG
KNLRIIRKGIHIIKKY(NH2) (SEQ ID NO: 4)) comprising the same
antimicrobial peptide and GGG linker showed 5-18% remaining activity
in the same assay.

| | | | % |
|---|---|---|---|
| | Amino Acid | | viability |
| Name | Sequence | SEQ ID NO | remaining |
| C16AG2 (N7,L14) | AFFRAFNRAFAQALAK | 5 | 16 |
| C16AG2 (T1) | TFFRAFARAFAQAAAK | 6 | 18 |
| C16AG2 (L14) | AFFRAFARAFAQALAK | 7 | 20 |
| C16AG2 (L5) | AFFRLFARAFAQAAAK | 8 | 21 |
| F2F6F10- | TLFRLLNRSLTQALGK | 9 | 26 |
| L2L6L10_C16G2 | | | |
| G15-F15_C16G2 | TFFRLFNRSFTQALFK | 10 | 29 |
| F10-L10_C16G2 | TFFRLFNRSLTQALGK | 11 | 30 |
| G15-N15_C16G2 | TFFRLFNRSFTQALNK | 12 | 30 |
| C16AG2 | AFFRAFARAFAQAAAK | 13 | 30 |
| C16AG2 (N7) | AFFRAFNRAFAQAAAK | 14 | 34 |
| G15-S15_C16G2 | TFFRLFNRSFTQALSK | 15 | 37 |
| C16AG2 (S9) | AFFRAFARSFAQAAAK | 16 | 38 |
| C16AG2 (G15) | AFFRAFARAFAQAAGK | 17 | 38 |
| C16AG2 (T11) | AFFRAFARAFTQAAAK | 18 | 39 |
| K16-Q16_C16G2 | TFFRLFNRSFTQALGQ | 19 | 42 |
| F6-L6_C16G2 | TFFRLLNRSFTQALGK | 20 | 43 |
| F2-W2_C16G2 | TWFRLFNRSFTQALGK | 21 | 45 |
| C16AG2 (F14) | AFFRAFARAFAQAFAK | 22 | 46 |
| F2 to Q2_C16G2 | TQFRLFNRSFTQALGK | 23 | 47 |
| G15-D15_C16G2 | TFFRLFNRSFTQALDK | 24 | 47 |
| G15-A15_C16G2 | TFFRLFNRSFTQALAK | 25 | 47 |
| K16-E16_C16G2 | TFFRLFNRSFTQALGE | 26 | 48 |
| N7-S7_C16G2 | TFFRLFSRSFTQALGK | 27 | 50 |
| K16-A16_C16G2 | TFFRLFNRSFTQALGA | 28 | 51 |
| N7-D7_C16G2 | TFFRLFDRSFTQALGK | 29 | 52 |
| K16-F16_C16G2 | TFFRLFNRSFTQALGF | 30 | 53 |
| C16AG2 (T1,S9,T11) | TFFRAFARSFTQAAAK | 31 | 56 |
| C16AG2 | TFFRLFARSFTQAAGK | 32 | 57 |
| (T1,L5,S9,T11,G15) | | | |
| ΔA13_ΔG15_C16G2 | TFFRLFNRSFTQLK | 33 | 57 |
| K16-S16_C16G2 | TFFRLFNRSFTQALGS | 34 | 59 |
| F2-L2_C16G2 | TLFRLFNRSFTQALGK | 35 | 63 |
| N7-F6/N21-I24 | TFFRLNFRSFTQALGK | 36 | 65 |
| F10 to Q10_C16G2 | TFFRLFNRSQTQALGK | 37 | 73 |
| Scan-16 | TFFRLFAAAFTQALGK | 38 | 73 |
| Scan-24 | TFFRLFNRSFTQALGK** | 39 | 75 |
| Scan-17 | TFFRLFNRSAAAALGK | 40 | 76 |
| N7-F10/N21-I32 | TFFRLFFRSNTQALGK*** | 41 | 76 |
| Scan-22 | TFFRLFNRSFTQPLGK | 42 | 77 |
| F2/6/10-A2/6/10_ | TAFRLANRSATQALGK | 43 | 78 |
| C16G2 | | | |
| Scan-18 | TFFRLFNRSFTQAAAA | 44 | 78 |
| F6 to Q6_C16G2 | TFFRLQNRSFTQALGK | 45 | 79 |
| Scan-23 | TFFRLFNRSFTQALPK | 46 | 79 |
| TFF-TYY_C16G2 | TYYRLFNRSFTQALGK | 47 | 80 |
| ΔN7_C16G2 | TFFRLF RSFTQALGK | 48 | 84 |
| F7/11/15 sub | TQFRLQNRSQTQALGK | 49 | 93 |
| Q_C16G2 | | | |

### Design and Construction of STAMPs and Other Chimeric Constructs.

In various embodiments, one or more targeting peptides described herein can be attached to one or more effectors (e.g., an antimicrobial peptide, an antibiotic, a ligand, a lipid or liposome, an agent that physically disrupts the extracellular matrix within a community of microorganisms, a detectable label, a porphyrin, a photosensitizing agent, an epitope tag, etc.) to form a chimeric constructs.

The effector comprises a moiety whose activity is typically to be delivered to the target microorganism(s), to a biofilm comprising the target microorganism(s), to a cell or tissue comprising the target microorganism(s), and the like.

In certain embodiments one or more targeting peptides are attached to a single effector. In certain embodiments one or more effectors are attached to a single targeting peptide. In certain embodiments multiple targeting peptides are attached to multiple effectors. The targeting peptide(s) can be attached directly to the effector(s) or through a linker. Where the targeting peptide and the effector comprise peptides the chimeric moiety can be a fusion protein.

Targeting Enhancers/Opsonins

In certain embodiments compositions are contemplated that incorporate a targeting enhancer (e.g., an opsonin) along with one or more targeting peptides. Targeting enhancers include moieties that increase binding affinity, and/or binding specificity, and/or internalization of a moiety by the target cell/microorganism.

Accordingly, in certain embodiments, a targeting peptide and/or a targeted antimicrobial molecule comprise a targeting peptide described herein attached (e.g., conjugated) to an opsonin. When bound to a target cell through the targeting peptide, the opsonin component encourages phagocytosis and destruction by resident macrophages, dendritic cells, monocytes, or PMNs. Opsonins contemplated for conjugation can be of a direct or indirect type.

Direct opsonins include, for example, any bacterial surface antigen, PAMP (pathogen-associated molecular pattern), or other molecule recognized by host PRRs (pathogen recognizing receptors). Opsonins can include, but are not limited to, bacterial protein, lipid, nucleic acid, carbohydrate and/or oligosaccharide moieties.

In certain embodiments opsonins include, but are not limited to, N-acetyl-D-glucosamine (GlcNAc), N-acetyl-D-galactosamine (GlaNAc), N-acetylglucosamine-containing muramyl peptides, NAG-muramyl peptides, NAG-NAM, peptidoglycan, teichoic acid, lipoteichoic acid, LPS, o-antigen, mannose, fucose, ManNAc, galactose, maltose, glucose, glucosamine, sucrose, mannosamine, galactose-alpha-1,3-galactosyl-beta-1,4-N-acetyl glucosamine, or alpha-1,3-gal-gal, or other sugars.

In certain embodiments, opsonins include indirect opsonins Indirect opsonins function through binding to a direct opsonin already present. For example an Fc portion of an antibody, a sugar-binding lectin protein (example MBL), or host complement factors (example C3b, C4b, iC3b).

In certain embodiments the opsonin is to galactose-alpha-1,3-galactosyl-beta-1,4-N-acetyl glucosamine, or alpha-1,3-gal-gal.

Other examples of opsonin molecules include, but are not limited to antibodies (e.g., IgG and IgA), components of the complement system (e.g., C3b, C4b, and iC3b), mannose-binding lectin (MBL) (initiates the formation of C3b), and the like.

Methods of coupling an opsonin to a targeting peptide are well known to those of skill in the art (see, e.g., discussion below regarding attachment of effectors to targeting peptides).

### Effectors.

Any of a wide number of effectors can be coupled to targeting peptides as described herein to preferentially deliver the effector to a target organism and/or tissue. Illustrative effectors include, but are not limited to detectable labels, small molecule antibiotics, antimicrobial peptides, porphyrins or other photosensitizers, epitope tags/antibodies for use in a pretargeting protocol, agents that physically disrupt the extracellular matrix within a community of microorganisms, microparticles and/or microcapsules, nanoparticles and/or nanocapsules, “carrier” vehicles including, but not limited to lipids, liposomes, dendrimers, cholic acid-based peptide mimics or other peptide mimics, steroid antibiotics, and the like.

Detectable Labels.

In certain embodiments chimeric moieties are provided comprising one or more targeting peptides (e.g., as described in Table 2) attached directly or through a linker to a detectable label. Such chimeric moieties are effective for detecting the presence and/or quantity, and/or location of the microorganism(s) (e.g., S. mutans) to which the targeting peptide is directed. Similarly these chimeric moieties are useful to identify cells and/or tissues and/or food stuffs and/or other compositions that are infected with the targeted microorganism(s).

Detectable labels suitable for use in such chimeric moieties include any composition detectable by spectroscopic, photochemical, biochemical, immunochemical, electrical, optical, or chemical means. Illustrative useful labels include, but are not limited to, biotin for staining with labeled streptavidin conjugates, avidin or streptavidin for labeling with biotin conjugates fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, ³²P, ⁹⁹Tc, ²⁰³Pb, ⁶⁷Ga, ⁶⁸Ga, ⁷²As, ¹¹¹In, ^{113m}In, ⁹⁷Ru, ⁶²Cu, ⁶⁴¹Cu, ⁵²Fe, ^{52m}Mn, ⁵¹Cr, ¹⁸⁶Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, ¹¹¹Ag, and the like), enzymes (e.g., horse radish peroxidase, alkaline phosphatase and others commonly used in an ELISA), various colorimetric labels, magnetic or paramagnetic labels (e.g., magnetic and/or paramagnetic nanoparticles), spin labels, radio-opaque labels, and the like. Patents teaching the use of such labels include, for example, U.S. Pat. Nos. 3,817,837; 3,850,752; 3,939,350; 3,996,345; 4,277,437; 4,275,149; and 4,366,241.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe—CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

In various embodiments spin labels are provided by reporter molecules with an unpaired electron spin which can be detected by electron spin resonance (ESR) spectroscopy. Illustrative spin labels include organic free radicals, transitional metal complexes, particularly vanadium, copper, iron, and manganese, and the like. Exemplary spin labels include, for example, nitroxide free radicals.

Means of detecting such labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence, e.g., by microscopy, visual inspection, via photographic film, by the use of electronic detectors such as charge coupled devices (CCDs) or photomultipliers and the like. Similarly, enzymatic labels may be detected by providing appropriate substrates for the enzyme and detecting the resulting reaction product. Finally, simple colorimetric labels may be detected simply by observing the color associated with the label.

Antibiotics.

In certain embodiments chimeric moieties are provided comprising one or more a targeting peptides (e.g. as described in Table 2) attached directly or through a linker to a small molecule antibiotic and/or to a carrier (e.g., a lipid or liposome, a polymer, etc.) comprising a small molecule antibiotic. Illustrative antibiotics are shown in Table 3.

TABLE 3
Illustrative antibiotics for use in the
chimeric moieties described herein.

| Class | Generic Name | BRAND NAME |
|---|---|---|
| Aminoglycosides | Amikacin | AMIKIN ® |
| | Gentamicin | GARAMYCIN ® |
| | Kanamycin | KANTREX ® |
| | Neomycin | |
| | Netilmicin | NETROMYCIN ® |
| | Streptomycin | |
| | Tobramycin | NEBCIN ® |
| | Paromomycin | HUMATIN ® |
| Carbacephem | Loracarbef | LORABID ® |
| Carbapenems | Ertapenem | INVANZ ® |
| | Doripenem | FINIBAX ® |
| | Imipenem/Cilastatin | PRIMAXIN ® |
| | Meropenem | MERREM ® |
| Cephalosporins | Cefadroxil | DURICEF ® |
| (First generation) | Cefazolin | ANCEF ® |
| | Cefalotin or Cefalothin | KEFLIN ® |
| | Cefalexin | KEFLEX ® |
| Cephalosporins | Cefaclor | CECLOR ® |
| (Second generation) | Cefamandole | MANDOLE ® |
| | Cefoxitin | MEFOXIN ® |
| | Cefprozil | CEFZIL ® |
| | Cefuroxime | CEFTIN, ZINNAT ® |
| Cephalosporins | Cefixime | SUPRAX ® |
| (Third generation) | Cefdinir | OMNICEF ® |
| | Cefditoren | SPECTRACEF ® |
| | Cefoperazone | CEFOBID ® |
| | Cefotaxime | CLAFORAN ® |
| | Cefpodoxime | |
| | Ceftazidime | FORTAZ ® |
| | Ceftibuten | CEDAX ® |
| | Ceftizoxime | |
| | Ceftriaxone | ROCEPHIN ® |
| Cephalosporins | Cefepime | MAXIPIME ® |
| (Fourth generation) | | |
| Cephalosporins | Ceftobiprole | |
| (Fifth generation) | | |
| Glycopeptides | Teicoplanin | |
| | Vancomycin | VANCOCIN ® |
| | Dalbavancin | |
| Macrolides | | |
| Azithromycin | Zithromax | |
| Clarithromycin | Biaxin | |
| Dirithromycin | | |
| Erythromycin | Erythocin, Erythroped | |
| Roxithromycin | | |
| Troleandomycin | | |
| Telithromycin | Ketek | |
| Monobactams | Aztreonam | |
| Penicillins | Amoxicillin | NOVAMOX ®, |
| | | AMOXIL ® |
| | Ampicillin | |
| | Azlocillin | |
| | Carbenicillin | |
| | Cloxacillin | |
| | Dicloxacillin | |
| | Flucloxacillin | FLOXAPEN ® |
| | Mezlocillin | |
| | Meticillin | |
| | Nafcillin | |
| | Oxacillin | |
| | Penicillin | |
| | Piperacillin | |
| | Ticarcillin | |
| Polypeptides | Bacitracin | |
| | Colistin | |
| | Polymyxin B | |
| Quinolones | Mafenide | |
| | Prontosil (archaic) | |
| | Sulfacetamide | |
| | Sulfamethizole | |
| | Sulfanilimide (archaic) | |
| | Sulfasalazine | |
| | Sulfisoxazole | |
| | Trimethoprim | BACTRIM ® |
| | Trimethoprim- | |
| | Sulfamethoxazole | |
| | (Co-trimoxazole) | |
| | (TMP-SMX) | |
| Tetracyclines | Demeclocycline | |
| | Doxycycline | VIBRAMYCIN ® |
| | Minocycline | MINOCIN ® |
| | Oxytetracycline | TERRACIN ® |
| | Tetracycline | SUMYCIN ® |
| Natural products | Antimicrobial herbal | |
| | extracts | |
| | Essential oils | |
| | Farnesol | |
| | Licorice root extracts | |
| | Glycyrrhizol A | |
| | Glycyrrhizol B | |
| | 6,8-diisoprenyl-5,7,4′- | |
| | trihydroxyisoflavone | |
| Others | Arsphenamine | SALVARSAN ® |
| | Chloramphenicol | CHLOROMYCETIN ® |
| | Clindamycin | CLEOCIN ® |
| | Lincomycin | |
| | Ethambutol | |
| | Fosfomycin | |
| | Fusidic acid | FUCIDIN ® |
| | Furazolidone | |
| | Isoniazid | |
| | Linezolid | ZYVOX ® |
| | Tedizolid | |
| | Metronidazole | FLAGYL ® |
| | Mupirocin | BACTROBAN ® |
| | Nitrofurantoin | MACRODANTIN ®, |
| | | MACROBID ® |
| | Platensimycin | |
| | Pyrazinamide | |
| | Quinupristin/Dalfopristin | SYNCERCID ® |
| | Rifampin or Rifampicin | |
| | Tinidazole | |
| | Artemisinin | |
| | Fidaxomicin | |
| Antifungals | Amphotericin B | |
| | Anidulafungin | |
| | Caspofungin acetate | |
| | Clotrimazole | |
| | Fluconazole | |
| | Flucytosine | |
| | Griseofulvin | |
| | Itraconazole | |
| | Ketoconazole | |
| | Micafungin | |
| | Miconazole | |
| | Nystatin | |
| | Pentamidine | |
| | Posaconazole | |
| | Terbinafine | |
| | Voriconazole | |
| Antimycobiotics | Aminosalicylic Acid | |
| | Capreomycin | |
| | Clofazimine | |
| | Cycloserine | |
| | Ethionamide | |
| | Rifabutin | |
| | Rifapentine | |
| Antivirals | Abacavir | |
| | Acyclovir | |
| | Adefovir | |
| | Amantadine | |
| | Atazanavir | |
| | Cidofovir | |
| | Darunavir | |
| | Didanosine | |
| | Docosanol | |
| | Efavirenz | |
| | Emtricitabine | |
| | Enfuvirtide | |
| | Entecavir | |
| | Etravirine | |
| | Famciclovir | |
| | Fomivirsen | |
| | Fosamprenavir | |
| | Foscarnet | |
| | Ganciclovir | |
| | Idoxuridine | |
| | Indinavir | |
| | Interferon alpha | |
| | Lamivudine | |
| | Lopinavir/ritonavir | |
| | Maraviroc | |
| | Nelfinavir | |
| | Nevirapine | |
| | Oseltamivir | |
| | Penciclovir | |
| | Peramivir | |
| | Raltegravir | |
| | Ribavirin | |
| | Rimantadine | |
| | Ritonavir | |
| | Saquinavir | |
| | Stavudine | |
| | Telbivudine | |
| | Tenofovir | |
| | Tipranavir | |
| | Trifluridine | |
| | Valacyclovir | |
| | Valganciclovir | |
| | Zanamivir | |
| | Zidovudine | |
| Anti-parasitics | Albendazole | |
| | Artesunate | |
| | Atovaquone | |
| | Bephenium | |
| | hydroxynaphthoate | |
| | Chloroquine | |
| | Dapsone | |
| | Diethyl-carbamazine | |
| | Diloxanide furoate | |
| | Eflornithine | |
| | Emetine HCl | |
| | Furazolidone | |
| | Ivermectin | |
| | Lindane | |
| | Mebendazole | |
| | Mefloquine | |
| | Melarsoprol | |
| | Miltefosine | |
| | Niclosamide | |
| | Nifurtimox | |
| | Nitazoxanide | |
| | Oxamniquine | |
| | Paromomycin | |
| | Permethrin | |
| | Piperazine | |
| | Praziquantel | |
| | Primaquine | |
| | Pyrantel pamoate | |
| | Pyrimethamine | |
| | Proguanil | |
| | Quinacrine HCl | |
| | Quinidine | |
| | Quinine | |
| | Sodium Stibogluconate | |
| | Spiramycin | |
| | Thiabendazole | |
| | Tinidazole | |

Porphyrins and Non-Porphyrin Photosensitizers.

In certain embodiments, the targeting peptides described herein (e.g., peptides shown in Table 2) can be attached to porphyrins and other photosensitizers. A photosensitizer is a drug or other chemical that increases photosensitivity of the organism (e.g., bacterium, yeast, fungus, etc.). Photosensitizers can be useful in photodynamic antimicrobial chemotherapy (PACT). In various embodiments PACT utilizes photosensitizers and light (e.g., visible, ultraviolet, infrared, etc.) in order to give a phototoxic response in the target organism(s), often via oxidative damage.

Currently, the major use of PACT is in the disinfection of blood products, particularly for viral inactivation, although more clinically-based protocols are used, e.g. in the treatment of oral infection or topical infection. The technique has been shown to be effective in vitro against bacteria (including drug-resistant strains), yeasts, viruses, parasites, and the like.

Attaching a targeting peptide described herein to the photosensitizer provides a means of specifically or preferentially targeting the photosensitizer(s) to particular species or strains(s) of microorganism (e.g., S. mutans).

A wide range of photosensitizers, both natural and synthetic are known to those of skill in the art (see, e.g., Wainwright (1998) J. Antimicrob. Chemotherap. 42: 13-28). Photosensitizers are available with differing physicochemical make-up and light-absorption properties. In various embodiments photosensitizers are usually aromatic molecules that are efficient in the formation of long-lived triplet excited states. In terms of the energy absorbed by the aromatic-system, this again depends on the molecular structure involved. For example, furocoumarin photosensitizers (psoralens) absorb relatively high energy ultraviolet (UV) light (c. 300-350 nm), whereas macrocyclic, heteroaromatic molecules such as the phthalocyanines absorb lower energy, near-infrared light.

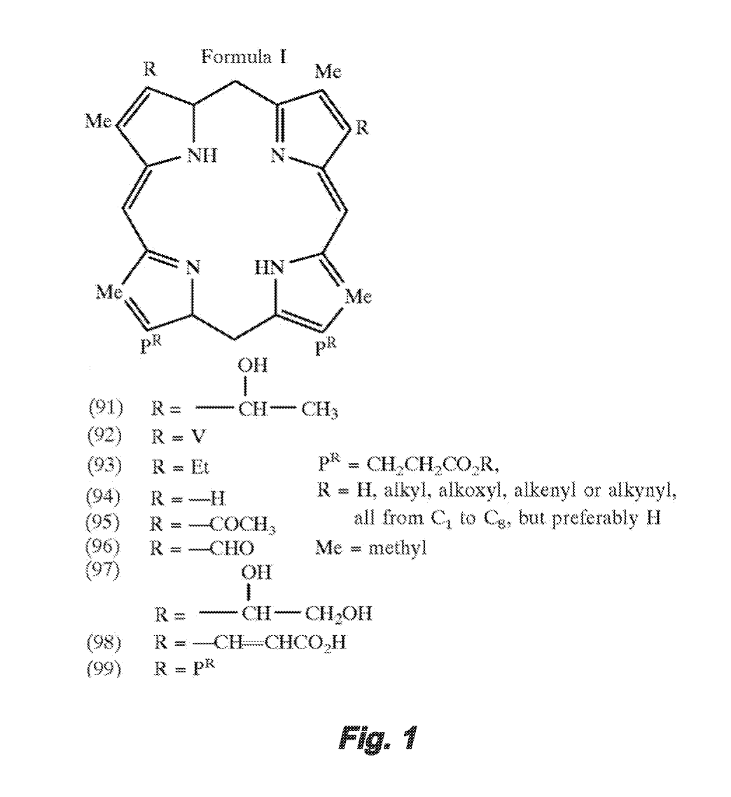

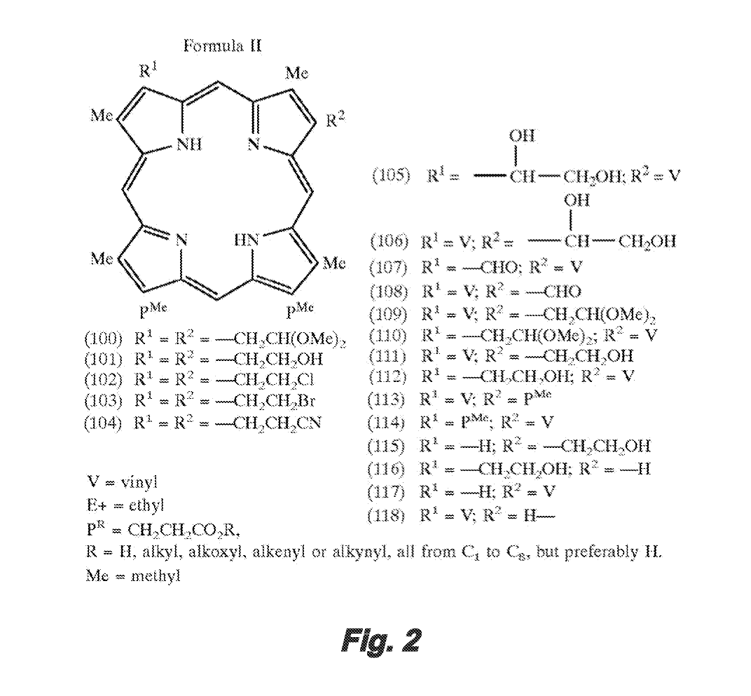

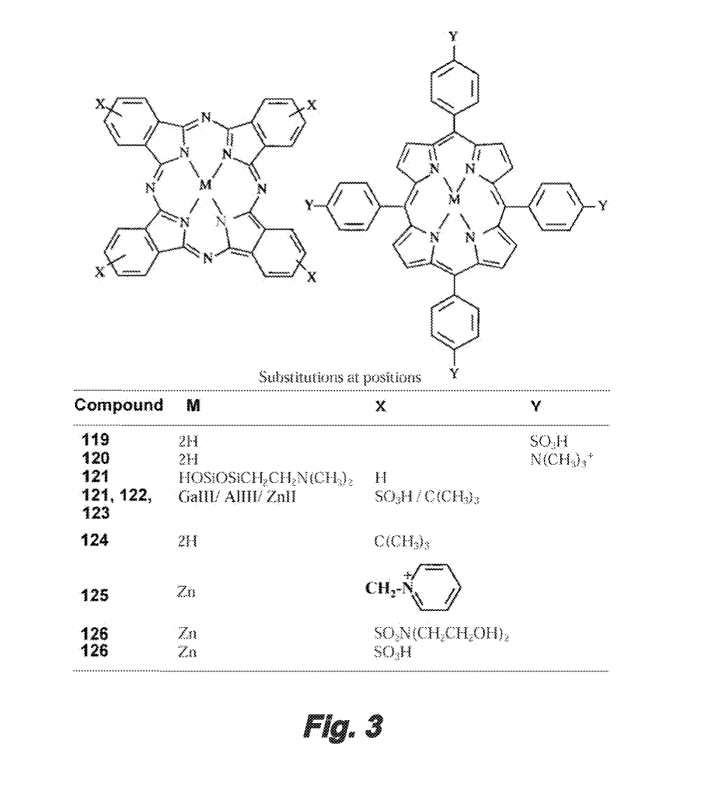

Illustrative photosensitizers include, but are not limited to porphyrinic macrocyles (especially porphyrins, chlorines, etc., see, e.g., FIGS. 1 and 2). In particular, metalloporphyrins, particularly a number of non-iron metalloporphyrins mimic haem in their molecular structure and are actively accumulated by bacteria via high affinity haem-uptake systems. The same uptake systems can be used to deliver antibiotic-porphyrin and antibacterial-porphyrin conjugates. Illustrative targeting porphyrins suitable for this purpose are described in U.S. Pat. No. 6,066,628 and shown herein in FIGS. 1 and 2.

An illustrative example of targeted porphyrins is shown in FIG. 13.

For example, certain artificial (non-iron) metalloporphyrins (MPs) (Ga-IX, Mn-IX,) are active against Gram-negative and Gram-positive bacteria and acid-fast bacilli (e.g., Y. enterocolitica, N. meningitides, S. marcescens, E. coli, P. mirabills, K. pneumoniae, K. oxytoca, Ps. aeruginosa, C. freundii, E. aerogenes, F. menigosepticum, S. aureus, B. subtilis, S. pyogenes A, E. faecalis, M. smegmatis, M. bovis, M. tuber., S. crevisiae) as described in Tables 1-5 of U.S. Pat. No. 6,066,628. These MPs can be used as targeting peptides against these microorganisms.

Similarly, some MPs are also growth-inhibitory against yeasts, indicating their usefulness targeting peptides to target Candida species (e.g., Candida albicans, C. krusei, C. pillosus, C. glabrata, etc.) and other mycoses including but not limited to those caused by as Trichophyton, Epidermophyton, Histoplasma, Aspergillus, Cryptococcus, and the like.

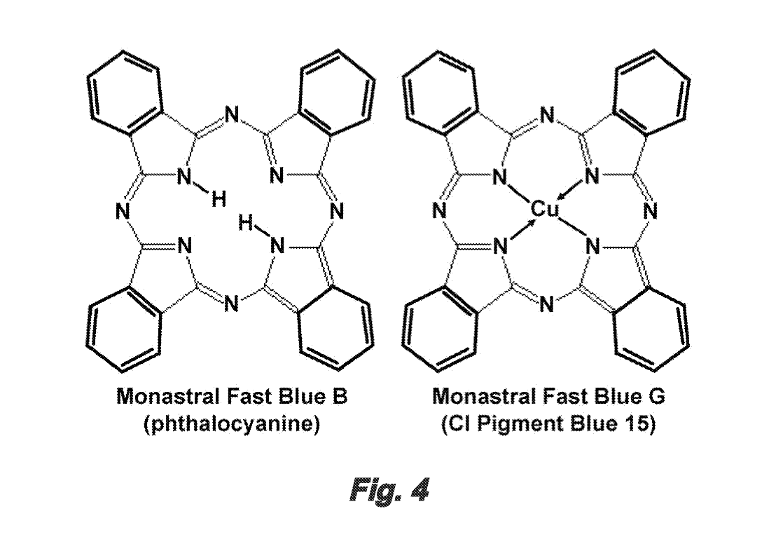

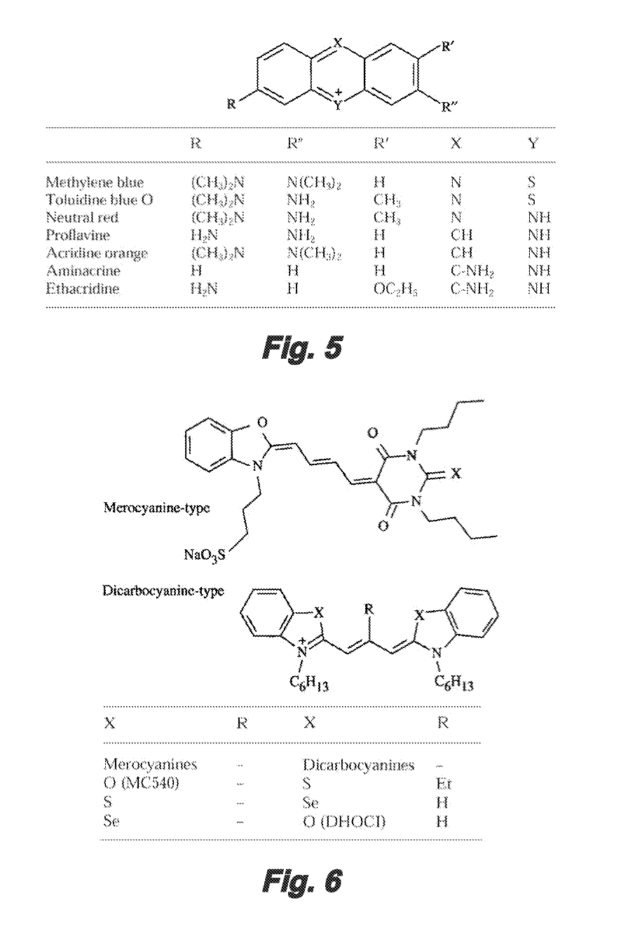

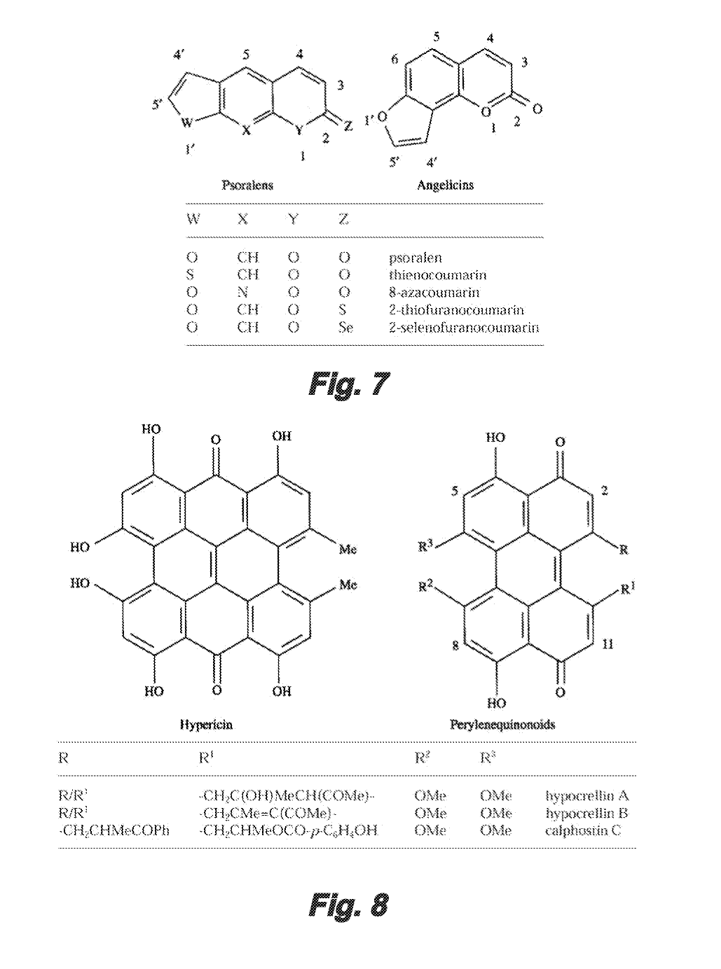

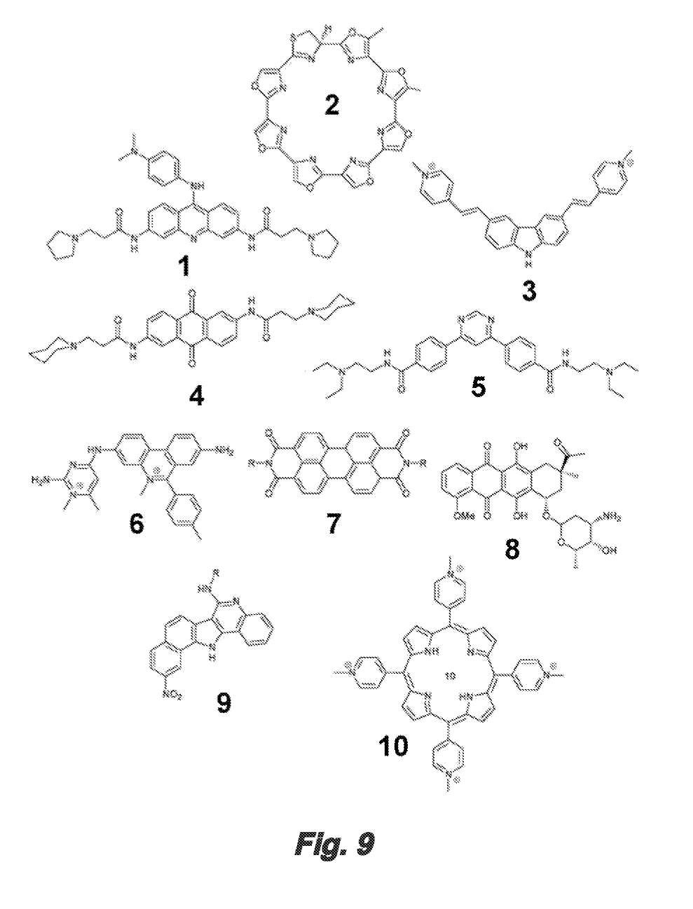

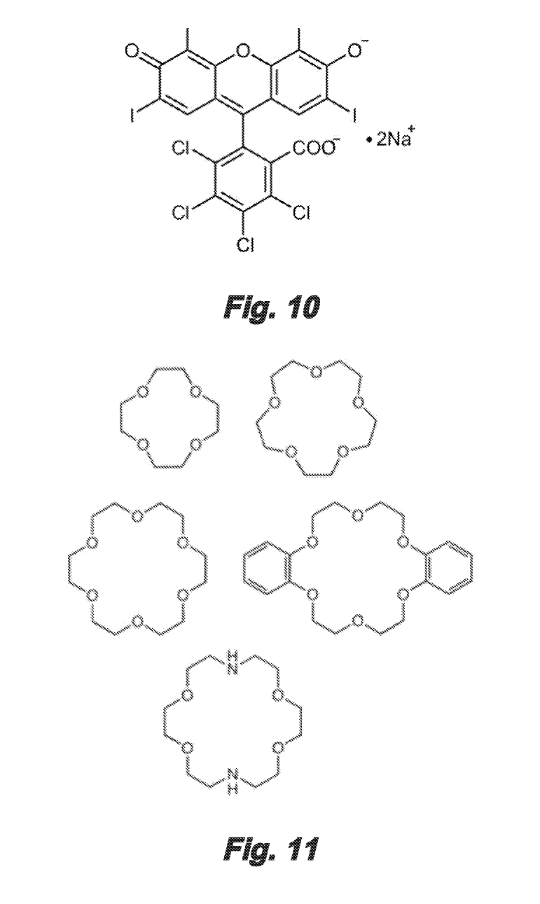

Other photosensitizers include, but are not limited to cyanines (see, e.g., FIG. 6) and phthalocyanines (see, e.g., FIG. 4), azines (see, e.g., FIG. 5) including especially methylene blue and touidine blue, hypericin (see, e.g., FIG. 8), acridines (see, e.g., FIG. 9) including especially Rose Bengal (see, e.g., FIG. 10), crown ethers (see, e.g., FIG. 11), and the like. In certain embodiments, the photosensitizers include tin chlorin 6 and related compounds (e.g., other chlorines and tin porphyrins).

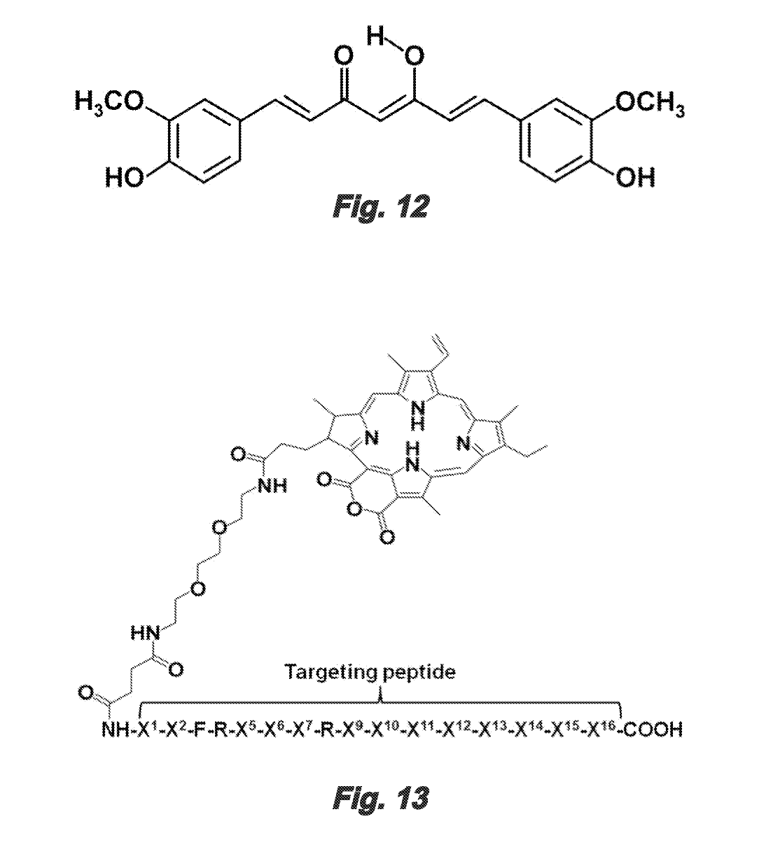

Another light-activated compound is cucumin (see, FIG. 12).

In certain embodiments the photosensitizers are toxic or growth inhibitors without light activation. For example, some non-iron metalloporphyrins (MPs) (see, e.g., FIGS. 1 and 2 herein) possess a powerful light-independent antimicrobial activity. In addition, haemin, the most well-known natural porphyrin, possesses a significant antibacterial activity that can be augmented by the presence of physiological concentrations of hydrogen peroxide or a reducing agent.

Typically when activated by light, the toxicity or growth inhibition effect is substantially increased. Typically, they generate radical species that affect anything within proximity. In certain embodiments to get the best selectivity from targeted photosensitizers, anti-oxidants can be used to quench un-bound photosensitizers, limiting the damage only to cells where the conjugates have accumulated due to the targeting peptide. The membrane structures of the target cell act as the proton donors in this case.

In typical photodynamic antimicrobial chemotherapy (PACT) the targeted photosensitizer is “activated by the application of a light source (e.g., a visible light source, an ultraviolet light source, an infrared light source, etc.). PACT applications however need not be limited to topical use. Regions of the mouth, throat, nose, sinuses are readily illuminated. Similarly regions of the gut can readily be illuminated using endoscopic techniques. Other internal regions can be illumined using laparoscopic methods or during other surgical procedures. For example, in certain embodiments involving the insertion or repair or replacement of an implantable device (e.g., a prosthetic device) it contemplated that the device can be coated or otherwise contacted with a chimeric moiety comprising a targeting peptide attached to a photosensitizer as described herein. During the surgical procedure and/or just before closing, the device can be illuminated with an appropriate light source to activate the photosensitizer.

The targeted photosensitizers and uses thereof described herein are illustrative and not to be limiting. Using the teachings provided herein, other targeted photosensitizers and uses thereof will be available to one of skill in the art.

Antimicrobial Peptides.

In certain embodiments, the targeting peptides described herein (e.g., peptides shown in Table 2) can be attached to one or more antimicrobial peptides to form selectively targeted antimicrobial peptides (STAMPs). Numerous antimicrobial peptides are well known to those of skill in the art.

In certain embodiments the antimicrobial peptides comprise one or more amino acid sequences described for example below in Table 4). In certain embodiments the antimicrobial peptides comprise one or more amino acid sequences described in the “Collection of Anti-Microbial Peptides” (CAMP) an online database developed for advancement the understanding of antimicrobial peptides (see, e.g., Thomas et al. (2009) Nucleic Acids Research, 2009, 1-7. doi:10.1093/nar/gkp1021) available at www.bicnirrh.res.in/antimicrobial.

TABLE 4
Novel antimicrobial peptides, target microorganisms and MIC values.

| | Organism | | SEQ |
|---|---|---|---|
| ID | MIC | Sequence | ID NO |
| G2 | | KNLRIIRKGIHIIKKY* | 3 |
| Novispirin | | KNLRRIIRKGIHIIKKYG | 50 |
| G10 | | | |
| Novispirin | | KNLRRIIRKTIHIIKKYG | 51 |
| T10 | | | |
| Novispirin G7 | | KNLRRIGRKIIHIIKKYG | 52 |
| Novispirin T7 | | KNLRRITRKIIHIIKKYG | 53 |
| Ovispirin | | KNLRRIIRKIIHIIKKYG | 54 |
| PGG | | GLLRRLRKKIGEIFKKYG | 55 |
| Protegrin-1 | | RGGRLCYCRRRFCVCVGR* | 56 |
| K-1 | S. mutans, 25 | GLGRVIGRLIKQIIWRR | 57 |
| | μM | | |
| K-2 | S. mutans, | VYRKRKSILKIYAKLKGWH | 58 |
| | 12.5 μM | | |
| K-7 | S. mutans, | NYRLVNAIFSKIFKKKFIKF | 59 |
| | 12.5 μM | | |
| K-8 | S. mutans, 4 | KILKFLFKKVF | 60 |
| | μM | | |
| K-9 | S. mutans, 4 | FIRKFLKKWLL | 61 |
| | μM | | |
| K-10 | S. mutans, 4 | KLFKFLRKHLL | 62 |
| | μM | | |
| K-11 | S. mutans, 4 | KILKFLFKQVF | 63 |
| | μM | | |
| K-12 | S. mutans, 8 | KILKKLFKFVF | 64 |
| | μM | | |
| K-13 | S. mutans, 16 | GILKKLFTKVF | 65 |
| | μM | | |
| K-14 | S. mutans, 8 | LRKFLHKLF | 66 |
| | μM | | |
| K-15 | S. mutans, 4 | LRKNLRWLF | 67 |
| | μM | | |
| K-16 | S. mutans, 8 | FIRKFLQKLHL | 68 |
| | μM | | |
| | P. | | |
| | aeruginosa, | | |
| | 12.5 μM | | |
| | MRSA, 25 | | |
| | μM | | |
| K-17 | S. mutans, 8 | FTRKFLKFLHL | 69 |
| | μM | | |
| K-18 | S. mutans, 16 | KKFKKFKVLKIL | 70 |
| | μM | | |
| K-19 | S. mutans, 16 | LLKLLKLKKLKF | 71 |
| | μM | | |
| K-20 | S. mutans, 8 | FLKFLKKFFKKLKY | 72 |
| | μM | | |
| K-21 | S. mutans, 8 | GWLKMFKKIIGKFGKF | 73 |
| | μM | | |
| K-22 | S. mutans, 8 | GIFKKFVKILYKVQKL | 74 |
| | μM | | |
| 1T-88 | | GRLVLEITADEVKALGEALANAKI | 75 |
| PF-531 | A. baumannii, | YIQFHLNQQPRPKVKKIKIFL-NH2 | 76 |
| | 25 μM | | |
| | P. | | |
| | aeruginosa, | | |
| | 50 μM | | |
| | T. rubrum, 50 | | |
| | μM | | |
| | A. niger, 25 | | |
| | μM | | |
| | B. subtilis, 25 | | |
| | μM | | |
| | C. difficile, | | |
| | 12.5 μM | | |
| | C. jeikeium, | | |
| | 6.25 μM | | |
| | S. | | |
| | epidermidis, | | |
| | 50 μM | | |
| | S. mutans, | | |
| | 12.5 μM | | |
| PF-527 | P. | GSVIKKRRKRMAKKKHRKLLKKTRIQR | 77 |
| | aeruginosa, | RRAGK | |
| | 50 μM | | |
| | T. rubrum, 25 | | |
| | μM | | |
| | A. niger, 50 | | |
| | μM | | |
| | B. subtilis, | | |
| | 12.5 μM | | |
| | C. jeikeium, | | |
| | 6.25 μM | | |
| | MRSA, 50 μM | | |
| | S. | | |
| | epidermidis, | | |
| | 25 μM | | |
| PF-672 | C. albicans, | MRFGSLALVAYDSAIKHSWPRPSSVRR | 78 |
| | 1.56 μM | LRM | |
| | T. rubrum, | | |
| | 0.78 μM | | |
| | A. niger, 3 | | |
| | μM | | |
| | B. subtilis, | | |
| | 0.78 μM | | |
| | E. faecalis, | | |
| | 3.13 μM | | |
| | MRSA, 1.56 | | |
| | μM | | |
| | S. | | |
| | epidermidis, | | |
| | 0.39 μM | | |
| PF-606 | E. coli, 50 μM | FESKILNASKELDKEKKVNTALSFNSHQ | 79 |
| | MRSA, 50 μM | DFAKAYQNGKI | |
| | S. | | |
| | epidermidis, | | |
| | 50 μM | | |
| | S. mutans, 50 | | |
| | μM | | |
| | S. | | |
| | pneumoniae, | | |
| | 50 μM | | |
| PF-547 | T. rubrum, 25 | WSRVPGHSDTGWKVWHRW-NH2 | 80 |
| | μM | | |
| | B. subtilis, 25 | | |
| | μM | | |
| | S. mutans, | | |
| | 12.5 μM | | |
| PF-006 | A. baumannii, | MGIIAGIIKFIKGLIEKFTGK | 81 |
| | 50 μM | | |
| | B. subtilis, 25 | | |
| | μM | | |
| | MRSA, 50 μM | | |
| PF-545 | A. niger, 50 | RESKLIAMADMIRRRI-NH2 | 82 |
| | μM | | |
| | B. subtilis, 25 | | |
| | μM | | |
| | MRSA, 50 μM | | |
| PF-278 | C. albicans, | LSLATFAKIFMTRSNWSLKRFNRL | 83 |
| | 50 μM | | |
| | T. rubrum, 50 | | |
| | μM | | |
| | S. | | |
| | epidermidis, | | |
| | 50 μM | | |
| PF-283 | T. rubrum, 50 | MIRIRSPTKKKLNRNSISDWKSNTSGRF | 84 |
| | μM | FY | |
| | B. subtilis, 50 | | |
| | μM | | |
| | S. | | |
| | epidermidis, | | |
| | 50 μM | | |
| PF-307 | C. albicans, | MKRRRCNWCGKLFYLEEKSKEAYCCK | 85 |
| | 50 μM | ECRKKAKKVKK | |
| | T. rubrum, 50 | | |
| | μM | | |
| | B. subtilis, 50 | | |
| | μM | | |
| PF-168 | T. rubrum, 50 | VLPFPAIPLSRRRACVAAPRPRSRQRAS | 86 |
| | μM | | |
| | A. niger, 50 | | |
| | μM | | |
| | MRSA, 50 μM | | |
| PF-538 | A. baumannii, | KNKKQTDILEKVKEILDKKKKTKSVGQ | 87 |
| | 25 μM | KLY | |
| | C. difficile, 25 | | |
| | μM | | |
| PF-448 | A. niger, 25 | SLQSQLGPCLHDQRH | 88 |
| | μM | | |
| | S. | | |
| | pneumoniae, | | |
| | 50 μM | | |
| PF-583 | MRSA, 50 μM | KFQGEFTNIGQSYIVSASHMSTSLNTGK | 89 |
| | S. | | |
| | epidermidis, | | |
| | 50 μM | | |
| PF-600 | E. coli, 50 μM | TKKIELKRFVDAFVKKSYENYILERELK | 90 |
| | S. | KLIKAINEELPTK | |
| | pneumoniae, | | |
| | 50 μM | | |
| PF-525 | A. niger, 50 | KFSDQIDKGQDALKDKLGDL | 91 |
| | μM | | |
| | S. | | |
| | pneumoniae, | | |
| | 50 μM | | |
| PF-529 | A. niger, 50 | LSEMERRRLRKRA-NH2 | 92 |
| | μM | | |
| | S. | | |
| | pneumoniae, | | |
| | 50 μM | | |
| PF-148 | A. niger, 50 | RRGCTERLRRMARRNAWDLYAEHFY | 93 |
| | μM | | |
| | B. subtilis, 50 | | |
| | μM | | |
| PF-530 | A. baumannii, | SKFKVLRKIIIKEYKGELMLSIQKQR | 94 |
| | 25 μM | | |
| PF-522 | C. difficile, 25 | FELVDWLETNLGKILKSKSA-NH2 | 95 |
| | μM | | |
| PF-497 | B. subtilis, 50 | LVLRICTDLFTFIKWTIKQRKS | 96 |
| | μM | | |
| PF-499 | B. subtilis, 50 | VYSFLYVLVIVRKLLSMKKRIERL | 97 |
| | μM | | |
| PF-322 | B. subtilis, 50 | GIVLIGLKLIPLLANVLR | 98 |
| | μM | | |
| PF-511 | S. | VMQSLYVKPPLILVTKLAQQN | 99 |
| | pneumoniae, | | |
| | 50 μM | | |
| PF-512 | S. | SFMPEIQKNTIPTQMK | 100 |
| | pneumoniae, | | |
| | 50 μM | | |
| PF-520 | S. | LGLTAGVAYAAQPTNQPTNQPTNQPTN | 101 |
| | pneumoniae, | QPTNQPTNQPRW-NH2 | |
| | 50 μM | | |
| PF-521 | S. | CGKLLEQKNFFLKTR | 102 |
| | pneumoniae, | | |
| | 50 μM | | |
| PF-523 | S. | ASKQASKQASKQASKQASKQASRSLKN | 103 |
| | pneumoniae, | HLL | |
| | 50 μM | | |
| PF-524 | S. | PDAPRTCYHKPILAALSRIVVTDR | 104 |
| | pneumoniae, | | |
| | 50 μM | | |
| PF-209 | MRSA, 50 μM | NYAVVSHT | 105 |
| PF-437 | S. pneumoniae, | FQKPFTGEEVEDFQDDDEIPTII | 106 |
| | 50 μM | | |
| CAM 135 | | GWRLIKKLLRVFKGL | 107 |
| B-33 | | FKKFWKWFRRF | 108 |
| B-34 | | LKRFLKWFKRF | 109 |
| B-35 | | KLFKRWKHLFR | 110 |
| B-36 | | RLLKRFKHLFK | 111 |
| B-37 | | FKTFLKWLHRF | 112 |
| B-38 | | IKQLLHFFQRF | 113 |
| B-39 | | KLLQTFKQIFR | 114 |
| B-40 | | RILKELKNLFK | 115 |
| B-41 | | LKQFVHFIHRF | 116 |
| B-42 | | VKTLLHIFQRF | 117 |
| B-43 | | KLVEQLKEIFR | 118 |
| B-44 | | RVLQEIKQILK | 119 |
| B-45 | | VKNLAELVHRF | 120 |
| B-46 | | ATHLLHALQRF | 121 |
| B-47 | | KLAENVKEILR | 122 |
| B-48 | | RALHEAKEALK | 123 |
| B-49 | | FHYFWHWFHRF | 124 |
| B-50 | | LYHFLHWFQRF | 125 |
| B-51 | | YLFQTWQHLFR | 126 |
| B-52 | | YLLTEFQHLFK | 127 |
| B-53 | | FKTFLQWLIIRF | 128 |
| B-54 | | IKTLLHFFQRF | 129 |
| B-55 | | KLLQTFNQIFR | 130 |
| B-56 | | TILQSLKNIFK | 131 |
| B-57 | | LKQFVKFIHRF | 132 |
| B-58 | | VKQLLKIFNRF | 133 |
| B-59 | | KLVQQLKNIFR | 134 |
| B-60 | | RVLNQVKQILK | 135 |
| B-61 | | VKKLAKLVRRF | 136 |
| B-62 | | AKRLLKVLKRF | 137 |
| B-63 | | KLAQKVKRVLR | 138 |
| B-64 | | RALKRIKHVLK | 139 |
| IC-1 | | RRRRWWW | 140 |
| IC-2 | | RRWWRRW | 141 |
| IC-3 | | RRRWWWR | 142 |
| IC-4 | | RWRWRWR | 143 |
| 2C-1 | | RRRFWWR | 144 |
| 2C-2 | | RRWWRRF* | 145 |
| 2C-3 | | RRRWWWF* | 146 |
| 2C-4 | | RWRWRWF* | 147 |
| 3C-1 | | RRRRWWK | 148 |
| 3C-2 | | RRWWRRK | 149 |
| 3C-3 | | RRRWWWK | 150 |
| 3C-4 | | RWRWRWK | 151 |
| 4C-1 | | RRRKWWK | 152 |
| 4C-2 | | RRWKRRK | 153 |
| 4C-3 | | RRRKWWK | 154 |
| 4C-4 | | RWRKRWK | 155 |
| a-3 | | LHLLHQLLHLLHQF* | 156 |
| a-4 | | AQAAHQAAHAAHQF* | 157 |
| a-5 | | KLKKLLKKLKKLLK | 158 |
| a-6 | | LKLLKKLLKLLKKF* | 159 |
| a-7 | | LQLLKQLLKLLKQF* | 160 |
| a-8 | | AQAAKQAAKAAKQF* | 161 |
| a-9 | | RWRRWWRHFHHFFH* | 162 |
| a-10 | | KLKKLLKRWRRWWR | 163 |
| a-11 | | RWRRLLKKLHHLLH* | 164 |
| a-12 | | KLKKLLKHLHHLLH* | 165 |
| BD-1 | | FVFRHKWVWKHRFLF | 166 |
| BD-2 | | VFIHRHVWVHKHVLF | 167 |
| BD-3 | | WRWRARWRWRLRWRF | 168 |
| BD-4 | | WRIHLRARLHVKFRF | 169 |
| BD-5 | | LRIHARFKVHIRLKF | 170 |
| BD-6 | | FHIKFRVHLKVRFHF | 171 |
| BD-7 | | FHVKIHFRLHVKFHF | 172 |
| BD-8 | | LHIHAHFHVHIHLHF | 173 |
| BD-9 | | FKIHFRLKVHIRFKF | 174 |
| BD-10 | | FKAHIRFKLRVKFHF | 175 |
| BD-11 | | LKAKIKFKVKLKIKF | 176 |
| BD-12 | | WIWKHKFLHRHFLF | 177 |
| BD-13 | | VFLHRHVIKHKLVF | 178 |
| BD-14 | | FLHKHVLRHRIVF | 179 |
| BD-15 | | VFKHKIVHRHILF | 180 |
| BD-16 | | FLFKHLFLHRIFF | 181 |
| BD-17 | | LFKHILIHRVIF | 182 |
| BD-18 | | FLHKHLFKHKLF | 183 |
| BD-19 | | VFRHRFIHRHVF | 184 |
| BD-20 | | FIHKLVHKHVLF | 185 |
| BD-21 | | VLRHLFRHRIVF | 186 |
| BD-22 | | LVHKLILRHLLF | 187 |
| BD-23 | | VFKRVLIHKLIF | 188 |
| BD-24 | | IVRKFLFRHKVF | 189 |
| BD-25 | | VLKHVIAHKRLF | 190 |
| BD-26 | | FIRKFLFKHLF | 191 |
| BD-27 | | VIRHVWVRKLF | 192 |
| BD-28 | | FLFRHRFRHRLVF | 193 |
| BD-29 | | LFLHKHAKHKFLF | 194 |
| BD-30 | | FKHKFKHKFIF | 195 |
| BD-31 | | LRHRLRHRLIF | 196 |
| BD-32 | | LILKFLFKFVF | 197 |
| BD-33 | | VLIRILVRVIF | 198 |
| BD-34 | | FRHRFRHRF | 199 |
| BD-35 | | LKHKLKHKF | 200 |
| BD-36 | | FKFKHKLIF | 201 |
| BD-37 | | LRLRHRVLF | 202 |
| BD-38 | | FKFLFKFLF | 203 |
| BD-39 | | LRLFLRWLF | 204 |
| BD-40 | | FKFLFKHKF | 205 |
| BD-41 | | LRLFLRHRF | 206 |
| BD-42 | | FKFLFKF | 207 |
| BD-43 | | LRLFLRF | 208 |
| AA-1 | | HHFFHHFHHFFHHF* | 209 |
| AA-2 | | FHFFHHFFHFFHHF* | 210 |
| AA-3 | | KLLKGATFHFFHHFFHFFHHF | 211 |
| AA-4 | | KLLKFHFFHHFFHFFHHF | 212 |
| AA-5 | | FHFFHHFFHFFHHFKLLK | 213 |
| RIP | | YSPWTNF* | 214 |
| LL-37 | | LLGDFFRKSKEKIGKEFKRIVQRIKDFLR | 215 |
| | | NLVPRTES | |
| Cys-LL-37 | | CLLGDFFRKSKEKIGKEFKRIVQRIKDFL | 216 |
| | | RNLVPRTES | |
| LL-37(17-32) | | FKRIVQRIKDFLRNLV | 217 |
| Cys-LL-37- | | CLLGDFFRKSKEKIGKEFKRIVQRIKDFL | 218 |
| Cys | | RNLVPRTESC | |
| LL-37FK-13 | | FKRIVQRIKDFLR | 219 |
| LL-37FKR | | FKRIVQRIKDFLRNLVPRTES | 220 |
| LL-37GKE | | GKEFKRIVQRIKDFLRNLVPR | 221 |
| LL-37KRI | | KRIVQRIKDFLRNLVPRTES | 222 |
| LL-37LLG | | LLGDFFRKSKEKIGKEFKRIV | 223 |
| LL-37RKS | | RKSKEKIGKEFKRIVQRIKDFLRNLVPR | 224 |
| | | TES | |
| LL-37SKE | | SKEKIGKEFKRIVQRIKDFLR | 225 |
| LL-37-Cys | | LLGDFFRKSKEKIGKEFKRIVQRIKDFLR | 226 |
| | | NLVPRTESC | |
| BD2.21 | | KLFKFLRKHLL | 227 |
| AF5 | | FLKFLKKFFKKLK | 228 |
| | | FIGAIARLLSKIFGKR-NH2 | 229 |
| | | GIFSKLAGKKIKNLLISG-NH2 | 230 |
| | | GIFSKLAGKKIKNLLISGLKG-NH2 | 231 |
| | | GLFSKFVGKGIKNFLIKGVK-NH2 | 232 |
| | | KAYSTPRCKGLFRALMCWL | 233 |
| | | KIFGAIWPLALGALKNLIK-NH₂ | 234 |
| | | GWGSFFKKAAHVGKHVGKAALTHYL- | 235 |
| | | NH2 | |
| | | RGLRRLGRKIAHGVKKYG-NH2 | 236 |
| | | RGLRRLGRKIAHGVKKYGPTVLRIIRIA | 237 |
| | | G | |
| | | KIAHGVKKYGPTVLRIIR | 238 |
| | | LLGDFFRKSKEKIGKEFKRIVQRIKDFLR | 239 |
| | | NLVPRTES | |
| | | FLPLIGRVLSGIL-NH₂ | 240 |
| | | IGKFLKKAKKFGKAFVKILKK-NH2 | 241 |
| | | GKFLKKAKKFGKAFVKIL-NH2 | 242 |
| | | WFLKFLKKFFKKLKY | 243 |
| | | RGLRRLGRKIAHGVKKY | 244 |
| | | LLGDFFRKSKEKI | 245 |
| | | ILRWPWWPWRRK-amide | 246 |

A number of antimicrobial peptides are also disclosed in U.S. Pat. Nos. 7,271,239, 7,223,840, 7,176,276, 6,809,181, 6,699,689, 6,420,116, 6,358,921, 6,316,594, 6,235,973, 6,183,992, 6,143,498, 6,042,848, 6,040,291, 5,936,063, 5,830,993, 5,428,016, 5,424,396, 5,032,574, 4,623,733, which are incorporated herein by reference for the disclosure of particular antimicrobial peptides.

In certain embodiments the antimicrobial peptides comprise one or more amino acid sequences described in the “Collection of Anti-Microbial Peptides” (CAMP) an online database developed for advancement the understanding of antimicrobial peptides (see, e.g., Thomas et al. (2009) Nucleic Acids Research, 2009, 1-7. doi:10.1093/nar/gkp1021) available at www.bicnirrh.res.in/antimicrobial.

In certain embodiments, the antimicrobial peptide is a novaspririn, a novaspirin fragment or analog, e.g., as shown above in Table 4. In certain embodiments constructs are contemplated where one or more of the targeting peptides described herein are attached (e.g., directly or through a linker) to a modulated version of novispirin G10 designated G2 (KNLRIIRKGIHIIKKY (SEQ ID NO:3). In this case, the C terminal amino acids are removed and an internal arginine is eliminated to facilitate chemical synthesis. Novispirin G10 (the “parent molecule”) is an antimicrobial alpha-helical octadecapeptide structurally related to cathelicidins and other innate immunity peptides.

Ligands.

In certain embodiments the effector can comprise one or more ligands, epitope tags, and/or antibodies. In certain embodiments preferred ligands and antibodies include those that bind to surface markers on immune cells. Chimeric moieties utilizing such antibodies as effector molecules act as bifunctional linkers establishing an association between the immune cells bearing binding partner for the ligand or antibody and the target microorganism(s).

The terms “epitope tag” or “affinity tag” are used interchangeably herein, and used refers to a molecule or domain of a molecule that is specifically recognized by an antibody or other binding partner. The term also refers to the binding partner complex as well. Thus, for example, biotin or a biotin/avidin complex are both regarded as an affinity tag. In addition to epitopes recognized in epitope/antibody interactions, affinity tags also comprise “epitopes” recognized by other binding molecules (e.g. ligands bound by receptors), ligands bound by other ligands to form heterodimers or homodimers, His₆ bound by Ni-NTA, biotin bound by avidin, streptavidin, or anti-biotin antibodies, and the like.

Epitope tags are well known to those of skill in the art. Moreover, antibodies specific to a wide variety of epitope tags are commercially available. These include but are not limited to antibodies against the DYKDDDDK (SEQ ID NO:247) epitope, c-myc antibodies (available from Sigma, St. Louis), the HNK-1 carbohydrate epitope, the HA epitope, the HSV epitope, the His₄ (SEQ ID NO:248), His₅ (SEQ ID NO:249), and His₆ (SEQ ID NO:250) epitopes that are recognized by the His epitope specific antibodies (see, e.g., Qiagen), and the like. In addition, vectors for epitope tagging proteins are commercially available. Thus, for example, the pCMV-Tag1 vector is an epitope tagging vector designed for gene expression in mammalian cells. A target gene inserted into the pCMV-Tag1 vector can be tagged with the FLAG® epitope (N-terminal, C-terminal or internal tagging), the c-myc epitope (C-terminal) or both the FLAG (N-terminal) and c-myc (C-terminal) epitopes.

Lipids and Liposomes.

In certain embodiments the targeting peptides described herein (e.g., the peptides shown in Table 2) are attached to one or more microparticles or nanoparticles that can be loaded with an effector agent (e.g., a pharmaceutical, a label, etc.). In certain embodiments the microparticles or nanoparticles are lipidic particles. Lipidic particles are microparticles or nanoparticles that include at least one lipid component forming a condensed lipid phase. Typically, a lipidic nanoparticle has preponderance of lipids in its composition. Various condensed lipid phases include solid amorphous or true crystalline phases; isomorphic liquid phases (droplets); and various hydrated mesomorphic oriented lipid phases such as liquid crystalline and pseudocrystalline bilayer phases (L-alpha, L-beta, P-beta, Lc), interdigitated bilayer phases, and nonlamellar phases (see, e.g., The Structure of Biological Membranes, ed. by P. Yeagle, CRC Press, Boca Raton, Fla., 1991). Lipidic microparticles include, but are not limited to a liposome, a lipid-nucleic acid complex, a lipid-drug complex, a lipid-label complex, a solid lipid particle, a microemulsion droplet, and the like. Methods of making and using these types of lipidic microparticles and nanoparticles, as well as attachment of affinity moieties, e.g., antibodies, to them are known in the art (see, e.g., U.S. Pat. Nos. 5,077,057; 5,100,591; 5,616,334; 6,406,713; 5,576,016; 6,248,363; Bondi et al. (2003) Drug Delivery 10: 245-250; Pedersen et al., (2006) Eur. J. Pharm. Biopharm. 62: 155-162, 2006 (solid lipid particles); U.S. Pat. Nos. 5,534,502; 6,720,001; Shiokawa et al. (2005) Clin. Cancer Res. 11: 2018-2025 (microemulsions); U.S. Pat. No. 6,071,533 (lipid-nucleic acid complexes), and the like).

A liposome is generally defined as a particle comprising one or more lipid bilayers enclosing an interior, typically an aqueous interior. Thus, a liposome is often a vesicle formed by a bilayer lipid membrane. There are many methods for the preparation of liposomes. Some of them are used to prepare small vesicles (d<0.05 micrometer), some for larger vesicles (d>0.05 micrometer). Some are used to prepare multilamellar vesicles, some for unilamellar ones. Methods for liposome preparation are exhaustively described in several review articles such as Szoka and Papahadjopoulos (1980) Ann. Rev. Biophys. Bioeng., 9: 467, Deamer and Uster (1983) Pp. 27-51 In: Liposomes, ed. M. J. Ostro, Marcel Dekker, New York, and the like.

In various embodiments the liposomes include a surface coating of a hydrophilic polymer chain. “Surface-coating” refers to the coating of any hydrophilic polymer on the surface of liposomes. The hydrophilic polymer is included in the liposome by including in the liposome composition one or more vesicle-forming lipids derivatized with a hydrophilic polymer chain. In certain embodiments, vesicle-forming lipids with diacyl chains, such as phospholipids, are preferred. One illustrative phospholipid is phosphatidylethanolamine (PE), which contains a reactive amino group convenient for coupling to the activated polymers. One illustrative PE is distearoyl PE (DSPE). Another example is non-phospholipid double chain amphiphilic lipids, such as diacyl- or dialkylglycerols, derivatized with a hydrophilic polymer chain.

In certain embodiments a hydrophilic polymer for use in coupling to a vesicle forming lipid is polyethyleneglycol (PEG), preferably as a PEG chain having a molecular weight between 1,000-10,000 Daltons, more preferably between 1,000-5,000 Daltons, most preferably between 2,000-5,000 Daltons. Methoxy or ethoxy-capped analogues of PEG are also useful hydrophilic polymers, commercially available in a variety of polymer sizes, e.g., 120-20,000 Daltons.

Other hydrophilic polymers that can be suitable include, but are not limited to polylactic acid, polyglycolic acid, polyvinylpyrrolidone, polymethyloxazoline, polyethyloxazoline, polyhydroxypropyl methacrylamide, polymethacrylamide, polydimethylacrylamide, and derivatized celluloses, such as hydroxymethylcellulose or hydroxyethylcellulose.

Preparation of lipid-polymer conjugates containing these polymers attached to a suitable lipid, such as PE have been described, for example in U.S. Pat. No. 5,395,

The liposomes can, optionally be prepared for attachment to one or more targeting peptides described herein. Here the lipid component included in the liposomes would include either a lipid derivatized with the targeting peptide, or a lipid having a polar-head chemical group, e.g., on a linker, that can be derivatized with the targeting peptide in preformed liposomes, according to known methods.

Methods of functionalizing lipids and liposomes with affinity moieties such as antibodies are well known to those of skill in the art (see, e.g., DE 3,218,121; Epstein et al. (1985) Proc. Natl. Acad. Sci., USA, 82:3688 (1985); Hwang et al. (1980) Proc. Natl. Acad. Sci., USA, 77: 4030; EP 52,322; EP 36,676; EP 88,046; EP 143,949; EP 142,641; Japanese patent application 83-118008; U.S. Pat. Nos. 4,485,045 and 4,544,545; and EP 102,324, all of which are incorporated herein by reference).

Agents that Physically Disrupt the Extracellular Matrix within a Community of Microorganisms

In certain embodiments the targeting peptides described herein (e.g., the peptides shown in Table 2) can be coupled to agents that physically disrupt the extracellular matrix within a community of microorganisms, for example a biofilm. In certain preferred embodiments, such an agent could be a bacterial cell-wall degrading enzyme, for example SAL-2, or Dispersin B, or any species of glycosidase, alginase, peptidase, proteinase, lipase, or DNA or RNA degrading enzyme or compound, for example rhRNase. Disruption of extracellular matrix of biofilms can result in clearance and therapeutic benefit.

The peptides can also be attached to antimicrobial proteins, such as Protein Inhibitor C or Colicin, or fragments thereof, for example the IIa domain of Colicin, or the heparin-binding domain of Protein Inhibitor C.

Polymeric Microparticles and/or Nanoparticles.

In certain embodiments the targeting peptides described herein (e.g., the peptides shown in Table 2) are attached to polymeric microparticles and/or nanoparticles and/or micelles.

Microparticle and nanoparticle-based drug delivery systems have considerable potential for treatment of various microorganisms. Technological advantages of polymeric microparticles or nanoparticles used as drug carriers are high stability, high carrier capacity, feasibility of incorporation of both hydrophilic and hydrophobic substances, and feasibility of variable routes of administration, including oral application and inhalation. Polymeric nanoparticles can also be designed to allow controlled (sustained) drug release from the matrix. These properties of nanoparticles enable improvement of drug bioavailability and reduction of the dosing frequency.

Polymeric nanoparticles are typically micron or submicron (<1 μm) colloidal particles. This definition includes monolithic nanoparticles (nanospheres) in which the drug is adsorbed, dissolved, or dispersed throughout the matrix and nanocapsules in which the drug is confined to an aqueous or oily core surrounded by a shell-like wall. Alternatively, in certain embodiments, the drug can be covalently attached to the surface or into the matrix.

Polymeric microparticles and nanoparticles are typically made from biocompatible and biodegradable materials such as polymers, either natural (e.g., gelatin, albumin) or synthetic (e.g., polylactides, polyalkylcyanoacrylates), or solid lipids. In the body, the drug loaded in nanoparticles is usually released from the matrix by diffusion, swelling, erosion, or degradation. One commonly used material is poly(lactide-co-glycolide) (PLG).

Methods of fabricating and loading polymeric nanoparticles or microparticles are well known to those of skill in the art. Thus, for example, Matsumoto et al. (1999) Intl. J. Pharmaceutics, 185: 93-101 teaches the fabrication of poly(L-lactide)—poly(ethylene glycol)—poly(L-lactide) nanoparticles, Chawla et al. (2002) Intl. J. Pharmaceutics 249: 127-138, teaches the fabrication and use of poly(e-caprolactone) nanoparticles delivery of tamifoxen, and Bodmeier et al. (1988) Intl. J. Pharmaceutics, 43: 179-186, teaches the preparation of poly(D,L-lactide) microspheres using a solvent evaporation method. “Intl. J. Pharmaceutics, 1988, 43, 179-186. Other nanoparticle formulations are described, for example, by Williams et al. (2003) J. Controlled Release, 91: 167-172; Leroux et al. (1996) J. Controlled Release, 39: 339-350; Soppimath et al. (2001) J. Controlled Release, 70: 1-20; Brannon-Peppas (1995) Intl. J. Pharmaceutics, 116: 1-9; and the like.

### Peptide Preparation.

The peptides described herein can be chemically synthesized using standard chemical peptide synthesis techniques or, particularly where the peptide does not comprise “D” amino acid residues, the peptide can be recombinantly expressed. Where the “D” polypeptides are recombinantly expressed, a host organism (e.g. bacteria, plant, fungal cells, etc.) can be cultured in an environment where one or more of the amino acids is provided to the organism exclusively in a D form. Recombinantly expressed peptides in such a system then incorporate those D amino acids.

In certain embodiments, D amino acids can be incorporated in recombinantly expressed peptides using modified amino acyl-tRNA synthetases that recognize D-amino acids.

In certain embodiments the peptides are chemically synthesized by any of a number of fluid or solid phase peptide synthesis techniques known to those of skill in the art. Solid phase synthesis in which the C-terminal amino acid of the sequence is attached to an insoluble support followed by sequential addition of the remaining amino acids in the sequence is a preferred method for the chemical synthesis of the polypeptides of this invention. Techniques for solid phase synthesis are well known to those of skill in the art and are described, for example, by Barany and Merrifield (1963) Solid-Phase Peptide Synthesis; pp. 3-284 in The Peptides: Analysis, Synthesis, Biology. Vol. 2: Special Methods in Peptide Synthesis, Part A.; Merrifield et al. (1963) J. Am. Chem. Soc., 85: 2149-2156, and Stewart et al. (1984) Solid Phase Peptide Synthesis, 2nd ed. Pierce Chem. Co., Rockford, Ill.

In one embodiment, the peptides can be synthesized by the solid phase peptide synthesis procedure using a benzhyderylamine resin (Beckman Bioproducts, 0.59 mmol of NH₂/g of resin) as the solid support. The COOH terminal amino acid (e.g., t-butylcarbonyl-Phe) is attached to the solid support through a 4-(oxymethyl)phenacetyl group. This is a more stable linkage than the conventional benzyl ester linkage, yet the finished peptide can still be cleaved by hydrogenation. Transfer hydrogenation using formic acid as the hydrogen donor can be used for this purpose.

It is noted that in the chemical synthesis of peptides, particularly peptides comprising D amino acids, the synthesis usually produces a number of truncated peptides in addition to the desired full-length product. Thus, the peptides are typically purified using, e.g., HPLC.

D-amino acids, beta amino acids, non-natural amino acids, and the like can be incorporated at one or more positions in the peptide simply by using the appropriately derivatized amino acid residue in the chemical synthesis. Modified residues for solid phase peptide synthesis are commercially available from a number of suppliers (see, e.g., Advanced Chem Tech, Louisville; Nova Biochem, San Diego; Sigma, St Louis; Bachem California Inc., Torrance, etc.). The D-form and/or otherwise modified amino acids can be completely omitted or incorporated at any position in the peptide as desired. Thus, for example, in certain embodiments, the peptide can comprise a single modified acid, while in other embodiments, the peptide comprises at least two, generally at least three, more generally at least four, most generally at least five, preferably at least six, more preferably at least seven or even all modified amino acids. In certain embodiments, essentially every amino acid is a D-form amino acid.

As indicated above, the peptides and/or fusion proteins of this invention can also be recombinantly expressed. Accordingly, in certain embodiments, the antimicrobial peptides and/or targeting peptides, and/or fusion proteins described herein are synthesized using recombinant expression systems. Generally this involves creating a DNA sequence that encodes the desired peptide or fusion protein, placing the DNA in an expression cassette under the control of a particular promoter, expressing the peptide or fusion protein in a host, isolating the expressed peptide or fusion protein and, if required, renaturing the peptide or fusion protein.

DNA encoding the peptide(s) or fusion protein(s) described herein can be prepared by any suitable method as described above, including, for example, cloning and restriction of appropriate sequences or direct chemical synthesis.

This nucleic acid can be easily ligated into an appropriate vector containing appropriate expression control sequences (e.g. promoter, enhancer, etc.), and, optionally, containing one or more selectable markers (e.g. antibiotic resistance genes).

The nucleic acid sequences encoding the peptides or fusion proteins described herein can be expressed in a variety of host cells, including, but not limited to, E. coli, other bacterial hosts, yeast, fungus, and various higher eukaryotic cells such as insect cells (e.g. SF3), the COS, CHO and HeLa cells lines and myeloma cell lines. The recombinant protein gene will typically be operably linked to appropriate expression control sequences for each host. For E. coli this can include a promoter such as the T7, trp, or lambda promoters, a ribosome binding site and preferably a transcription termination signal. For eukaryotic cells, the control sequences can include a promoter and often an enhancer (e.g., an enhancer derived from immunoglobulin genes, SV40, cytomegalovirus, etc.), and a polyadenylation sequence, and may include splice donor and acceptor sequences.

The plasmids can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant peptide(s) or fusion protein(s) can be purified according to standard procedures of the art, including ammonium sulfate precipitation, affinity columns, column chromatography, gel electrophoresis and the like (see, generally, R. Scopes, (1982) Protein Purification, Springer-Verlag, N.Y.; Deutscher (1990) Methods in Enzymology Vol. 182: Guide to Protein Purification., Academic Press, Inc. N.Y.). Substantially pure compositions of at least about 90 to 95% homogeneity are preferred, and 98 to 99% or more homogeneity are most preferred.

One of skill in the art would recognize that after chemical synthesis, biological expression, or purification, the peptide(s) or fusion protein(s) may possess a conformation substantially different than desired native conformation. In this case, it may be necessary to denature and reduce the peptide or fusion protein and then to cause the molecule to re-fold into the preferred conformation. Methods of reducing and denaturing proteins and inducing re-folding are well known to those of skill in the art (see, e.g., Debinski et al. (1993) J. Biol. Chem., 268: 14065-14070; Kreitman and Pastan (1993) Bioconjug. Chem., 4: 581-585; and Buchner, et al., (1992) Anal. Biochem., 205: 263-270). Debinski et al., for example, describes the denaturation and reduction of inclusion body proteins in guanidine-DTE. The protein is then refolded in a redox buffer containing oxidized glutathione and L-arginine.

One of skill would recognize that modifications can be made to the peptide(s) and/or fusion protein(s) proteins without diminishing their biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

### Joining Targeting Peptides to Other Moieties.

Chemical Conjugation.

Chimeric moieties are formed by joining one or more of the targeting peptides described herein to one or more effectors. In certain embodiments the targeting peptides are attached directly to the effector(s) via naturally occurring reactive groups or the targeting peptide(s) and/or the effector(s) can be functionalized to provide such reactive groups.

In various embodiments the targeting peptides are attached to effector(s) via one or more linking agents. Thus, in various embodiments the targeting peptides and the effector(s) can be conjugated via a single linking agent or multiple linking agents. For example, the targeting peptide and the effector can be conjugated via a single multifunctional (e.g., bi-, tri-, or tetra-) linking agent or a pair of complementary linking agents. In another embodiment, the targeting peptide and the effector are conjugated via two, three, or more linking agents. Suitable linking agents include, but are not limited to, e.g., functional groups, affinity agents, stabilizing groups, and combinations thereof.

In certain embodiments the linking agent is or comprises a functional group. Functional groups include monofunctional linkers comprising a reactive group as well as multifunctional crosslinkers comprising two or more reactive groups capable of forming a bond with two or more different functional targets (e.g., labels, proteins, macromolecules, semiconductor nanocrystals, or substrate). In some preferred embodiments, the multifunctional crosslinkers are heterobifunctional crosslinkers comprising two or more different reactive groups.

Suitable reactive groups include, but are not limited to thiol (—SH), carboxylate (COOH), carboxyl (—COOH), carbonyl, amine (NH₂), hydroxyl (—OH), aldehyde (—CHO), alcohol (ROH), ketone (R₂CO), active hydrogen, ester, sulfhydryl (SH), phosphate (—PO₃), or photoreactive moieties. Amine reactive groups include, but are not limited to e.g., isothiocyanates, isocyanates, acyl azides, NHS esters, sulfonyl chlorides, aldehydes and glyoxals, epoxides and oxiranes, carbonates, arylating agents, imidoesters, carbodiimides, and anhydrides. Thiol-reactive groups include, but are not limited to e.g., haloacetyl and alkyl halide derivates, maleimides, aziridines, acryloyl derivatives, arylating agents, and thiol-disulfides exchange reagents. Carboxylate reactive groups include, but are not limited to e.g., diazoalkanes and diazoacetyl compounds, such as carbonyldiimidazoles and carbodiimides. Hydroxyl reactive groups include, but are not limited to e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, and isocyanates. Aldehyde and ketone reactive groups include, but are not limited to e.g., hydrazine derivatives for schiff base formation or reduction amination. Active hydrogen reactive groups include, but are not limited to e.g., diazonium derivatives for mannich condensation and iodination reactions. Photoreactive groups include, but are not limited to e.g., aryl azides and halogenated aryl azides, benzophenones, diazo compounds, and diazirine derivatives.

Other suitable reactive groups and classes of reactions useful in forming chimeric moieties include those that are well known in the art of bioconjugate chemistry. Currently favored classes of reactions available with reactive chelates are those which proceed under relatively mild conditions. These include, but are not limited to, nucleophilic substitutions (e.g., reactions of amines and alcohols with acyl halides, active esters), electrophilic substitutions (e.g., enamine reactions), and additions to carbon-carbon and carbon-heteroatom multiple bonds (e.g., Michael reaction, Diels-Alder addition). These and other useful reactions are discussed in, for example, March (1985) Advanced Organic Chemistry, 3rd Ed., John Wiley & Sons, New York, Hermanson (1996) Bioconjugate Techniques, Academic Press, San Diego; and Feeney et al. (1982) Modification of Proteins; Advances in Chemistry Series, Vol. 198, American Chemical Society, Washington, D.C.

In certain embodiments, the linking agent comprises a chelator. For example, the chelator comprising the molecule, DOTA (DOTA=1,4,7,10-tetrakis(carboxymethyl)-1,4,7,10-tetraazacyclododecane), can readily be labeled with a radiolabel, such as Gd³⁺ and ⁶⁴Cu, resulting in Gd³⁺-DOTA and ⁶⁴Cu-DOTA respectively, attached to the targeting peptide. Other suitable chelates are known to those of skill in the art, for example, 1,4,7-triazacyclononane-N,N′,N″-triacetic acid (NOTA) derivatives being among the most well-known (see, e.g., Lee et al. (1997) Nucl. Med. Biol. 24: 2225-23019).

A “linker” or “linking agent” as used herein, is a molecule that is used to join two or more molecules. In certain embodiments the linker is typically capable of forming covalent bonds to both molecule(s) (e.g., the targeting peptide and the effector). Suitable linkers are well known to those of skill in the art and include, but are not limited to, straight or branched-chain carbon linkers, heterocyclic carbon linkers, or peptide linkers. In certain embodiments the linkers can be joined to the constituent amino acids through their side groups (e.g., through a disulfide linkage to cysteine). However, in certain embodiments, the linkers will be joined to the alpha carbon amino and carboxyl groups of the terminal amino acids.

A bifunctional linker having one functional group reactive with a group on one molecule (e.g., a targeting peptide), and another group reactive on the other molecule (e.g., an antimicrobial peptide), can be used to form the desired conjugate. Alternatively, derivatization can be performed to provide functional groups. Thus, for example, procedures for the generation of free sulfhydryl groups on peptides are also known (See U.S. Pat. No. 4,659,839).

In certain embodiments the linking agent is a heterobifunctional crosslinker comprising two or more different reactive groups that form a heterocyclic ring that can interact with a peptide. For example, a heterobifunctional crosslinker such as cysteine may comprise an amine reactive group and a thiol-reactive group can interact with an aldehyde on a derivatized peptide. Additional combinations of reactive groups suitable for heterobifunctional crosslinkers include, for example, amine- and sulfhydryl reactive groups; carbonyl and sulfhydryl reactive groups; amine and photoreactive groups; sulfhydryl and photoreactive groups; carbonyl and photoreactive groups; carboxylate and photoreactive groups; and arginine and photoreactive groups. In one embodiment, the heterobifunctional crosslinker is SMCC.

Many procedures and linker molecules for attachment of various molecules to peptides or proteins are known (see, e.g., European Patent Application No. 188,256; U.S. Pat. Nos. 4,671,958, 4,659,839, 4,414,148, 4,699,784; 4,680,338; 4,569,789; and 4,589,071; and Borlinghaus et al. (1987) Cancer Res. 47: 4071-4075). Illustrative linking protocols are provided herein in Examples 2 and 3.

Fusion Proteins.

In certain embodiments where the targeting peptide and the moiety to be attached are both peptides or both comprise peptides, the chimeric moiety can be chemically synthesized or expressed as a recombinant fusion protein (i.e., a chimeric fusion protein).

In certain embodiments the chimeric fusion proteins are synthesized using recombinant DNA methodology. Generally this involves creating a DNA sequence that encodes the fusion protein, placing the DNA in an expression cassette under the control of a particular promoter, expressing the protein in a host, isolating the expressed protein and, if required, renaturing the protein.

DNA encoding the fusion proteins can be prepared by any suitable method, including, for example, cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066.

Chemical synthesis produces a single stranded oligonucleotide. This can be converted into double stranded DNA by hybridization with a complementary sequence or by polymerization with a DNA polymerase using the single strand as a template. One of skill would recognize that while chemical synthesis of DNA is limited to sequences of about 100 bases, longer sequences can be obtained by the ligation of shorter sequences.

Alternatively, subsequences can be cloned and the appropriate subsequences cleaved using appropriate restriction enzymes. The fragments can then be ligated to produce the desired DNA sequence.

In certain embodiments, DNA encoding fusion proteins of the present invention may be cloned using DNA amplification methods such as polymerase chain reaction (PCR). Thus, for example, the nucleic acid encoding a targeting antibody, a targeting peptide, and the like is PCR amplified, using a sense primer containing the restriction site for NdeI and an antisense primer containing the restriction site for HindIII. This produces a nucleic acid encoding the targeting sequence and having terminal restriction sites. Similarly an effector and/or effector/linker/spacer can be provided having complementary restriction sites. Ligation of sequences and insertion into a vector produces a vector encoding the fusion protein.

While the targeting peptides and other moieties (e.g., AMPs) can be directly joined together, one of skill will appreciate that they can be separated by a peptide spacer/linker consisting of one or more amino acids. Generally the spacer will have no specific biological activity other than to join the proteins or to preserve some minimum distance or other spatial relationship between them. However, the constituent amino acids of the spacer may be selected to influence some property of the molecule such as the folding, net charge, or hydrophobicity.

The nucleic acid sequences encoding the fusion proteins can be expressed in a variety of host cells, including E. coli, other bacterial hosts, yeast, and various higher eukaryotic cells such as the COS, CHO and HeLa cells lines and myeloma cell lines. The recombinant protein gene will be operably linked to appropriate expression control sequences for each host. For E. coli this includes a promoter such as the T7, trp, or lambda promoters, a ribosome binding site and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences.

The plasmids can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant fusion proteins can be purified according to standard procedures of the art, including ammonium sulfate precipitation, affinity columns, column chromatography, gel electrophoresis and the like (see, generally, R. Scopes (1982) Protein Purification, Springer-Verlag, N.Y.; Deutscher (1990) Methods in Enzymology Vol. 182: Guide to Protein Purification., Academic Press, Inc. N.Y.). Substantially pure compositions of at least about 90 to 95% homogeneity are preferred, and 98 to 99% or more homogeneity are most preferred for pharmaceutical uses. Once purified, partially or to homogeneity as desired, the polypeptides may then be used therapeutically.

One of skill in the art would recognize that after chemical synthesis, biological expression, or purification, the fusion protein may possess a conformation substantially different than the native conformations of the constituent polypeptides. In this case, it may be necessary to denature and reduce the polypeptide and then to cause the polypeptide to re-fold into the preferred conformation. Methods of reducing and denaturing proteins and inducing re-folding are well known to those of skill in the art (See, Debinski et al. (1993) J. Biol. Chem., 268: 14065-14070; Kreitman and Pastan (1993) Bioconjug. Chem., 4: 581-585; and Buchner, et al. (1992) Anal. Biochem., 205: 263-270).

One of skill would recognize that modifications can be made to the fusion proteins without diminishing their biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids placed on either terminus to create conveniently located restriction sites or termination codons.

As indicated above, in various embodiments a peptide linker/spacer is used to join the one or more targeting peptides to one or more effector(s). In various embodiments the peptide linker is relatively short, typically less than about 10 amino acids, preferably less than about 8 amino acids and more preferably about 3 to about 5 amino acids. Suitable illustrative linkers include, but are not limited to PSGSP ((SEQ ID NO:251), ASASA (SEQ ID NO: 252), or GGG. In certain embodiments longer linkers such as (GGGGS)₃ (SEQ ID NO:253) can be used. Illustrative peptide linkers and other linkers are shown in Table 5.

TABLE 5
Illustrative peptide and non-peptide linkers .

| Linker | SEQ ID NO: |
|---|---|
| AAA | |
| GGG | |
| SGG | |
| SAT | |
| PYP | |
| ASA | |
| GGGG | 254 |
| PSPSP | 255 |
| PSPSP | 256 |
| KKKK | 257 |
| RRRR | 258 |
| ASASA | 259 |
| GGSGGS | 260 |
| GGGGS | 261 |
| GGGGS GGGGS | 262 |
| GGGGS GGGGS GGGGS | 263 |
| GGGGS GGGGS GGGGS GGGGS | 264 |
| GGGGS GGGGS GGGGS GGGGS GGGGS | 265 |
| GGGGS GGGGS GGGGS GGGGS GGGGS GGGGS | 266 |
| 2-nitrobenzene or O-nitrobenzyl | |
| Nitropyridyl disulfide | |
| Dioleoylphosphatidylethanolamine (DOPE) | |
| S-acetylmercaptosuccinic acid | |
| 1, 4, 7, 10-tetraazacyclododecane-1, 4, 7, 10-tetracetic | |
| acid (DOTA) | |
| β-glucuronide and β-glucuronide variants | |
| Poly(alkylacrylic acid) | |
| Benzene-based linkers (for example: 2,5-Bis(hexyloxy)- | |
| 1,4-bis[2,5-bis(hexyloxy)-4-formyl-phenylenevinylene] | |
| benzene) and like molecules | |
| Disulfide linkages | |
| Poly(amidoamine) or like dendrimers linking multiple | |
| target and killing peptides in one molecule | |
| Carbon nanotubes | |
| Hydrazone and hydrazone variant linkers | |
| PEG of any chain length | |
| Succinate, formate, acetate butyrate, other like organic | |
| acids | |
| Aldols, alcohols, or enols | |
| Peroxides | |
| alkane or alkene groups of any chain length | |
| One or more porphyrin or dye molecules containing free | |
| amide and carboxylic acid groups | |
| One or more DNA or RNA nucleotides, including polyamine | |
| and polycarboxyl-containing variants | |
| Inulin, sucrose, glucose, or other single, di or | |
| polysaccharides | |
| Linoleic acid or other polyunsaturated fatty acids | |
| Variants of any of the above linkers containing halogen | |
| or thiol groups | |

(All amino-acid-based linkers could be L, D, combinations of L and D forms, (β-form, and the like)

Multiple Targeting Peptides and/or Effectors.

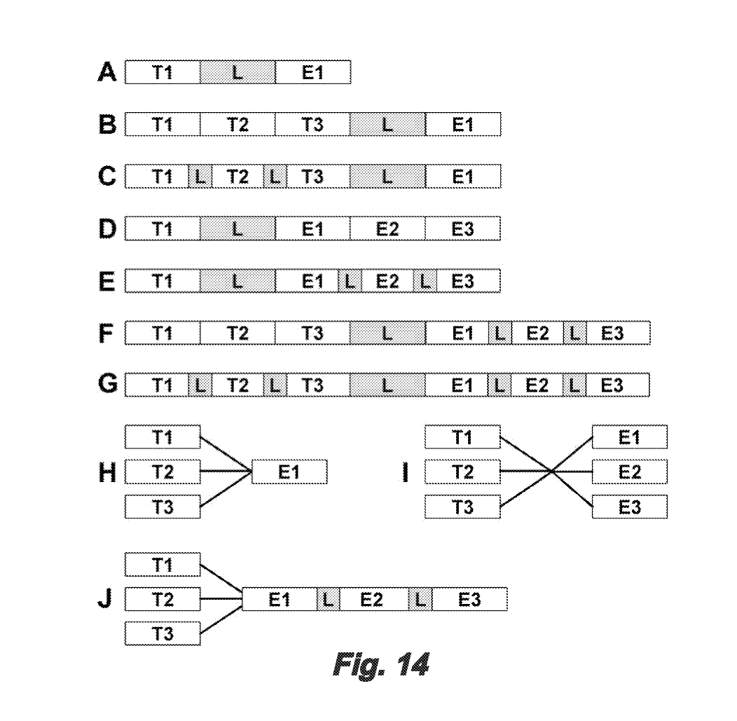

As indicated above, in certain embodiments, the chimeric moieties described herein can comprise multiple targeting peptides attached to a single effector or multiple effectors attached to a single targeting peptide, or multiple targeting peptides attached to multiple effectors.

Where the chimeric construct is a fusion protein this is easily accomplished by providing multiple domains that are targeting domains attached to one or more effector domains. FIG. 14 schematically illustrates a few, but not all, configurations. In various embodiments the multiple targeting domains and/or multiple effector domains can be attached to each other directly or can be separated by linkers (e.g., amino acid or peptide linkers as described above).

When the chimeric construct is a chemical conjugate linear or branched configurations (e.g., as illustrated in FIG. 14) are readily produced by using branched or multifunctional linkers and/or a plurality of different linkers.

Protecting Groups.

While the various peptides described herein may be shown with no protecting groups, in certain embodiments they can bear one, two, three, four, or more protecting groups. In various embodiments, the protecting groups can be coupled to the C- and/or N-terminus of the peptide(s) and/or to one or more internal residues comprising the peptide(s) (e.g., one or more R-groups on the constituent amino acids can be blocked). Thus, for example, in certain embodiments, any of the peptides described herein can bear, e.g., an acetyl group protecting the amino terminus and/or an amide group protecting the carboxyl terminus. Examples of such protected peptides include AFFRAFNRAFAQALAKGGGKNLRIIRKGIHIIKKY* (SEQ ID NO:267), TFFRAFARAFAQAAAKGGGKNLRIIRKGIHIIKKY* (SEQ ID NO:268), AFFRAFARAFAQALAKGGGKNLRIIRKGIHIIKKY* (SEQ ID NO:269), AFFRLFARAFAQAAAKGGGKNLRIIRKGIHIIKKY* (SEQ ID NO:270), TLFRLLNRSLTQALGKGGGKNLRIIRKGIHIIKKY* (SEQ ID NO:271), TFFRLFNRSFTQALFKGGGKNLRIIRKGIHIIKKY* (SEQ ID NO:272), TFFRLFNRSLTQALGKGGGKNLRIIRKGIHIIKKY* (SEQ ID NO:273), TFFRLFNRSFTQALNKGGGKNLRIIRKGIHIIKKY* (SEQ ID NO:274), AFFRAFARAFAQAAAKGGGKNLRIIRKGIHIIKKY* (SEQ ID NO:275), AFFRAFNRAFAQAAAKGGGKNLRIIRKGIHIIKKY* (SEQ ID NO:276), TFFRLFNRSFTQALSKGGGKNLRIIRKGIHIIKKY* (SEQ ID NO:277), AFFRAFARSFAQAAAKGGGKNLRIIRKGIHIIKKY* (SEQ ID NO:278), AFFRAFARAFAQAAGKGGGKNLRIIRKGIHIIKKY* (SEQ ID NO:279), AFFRAFARAFTQAAAKGGGKNLRIIRKGIHIIKKY* (SEQ ID NO:280), TFFRLFNRSFTQALGQGGGKNLRIIRKGIHIIKKY* (SEQ ID NO:281), TFFRLLNRSFTQALGKGGGKNLRIIRKGIHIIKKY* (SEQ ID NO:282), TWFRLFNRSFTQALGKGGGKNLRIIRKGIHIIKKY* (SEQ ID NO:283), AFFRAFARAFAQAFAKGGGKNLRIIRKGIHIIKKY* (SEQ ID NO:284), TQFRLFNRSFTQALGKGGGKNLRIIRKGIHIIKKY* (SEQ ID NO:285), TFFRLFNRSFTQALDKGGGKNLRIIRKGIHIIKKY* (SEQ ID NO:286), TFFRLFNRSFTQALAKGGGKNLRIIRKGIHIIKKY* (SEQ ID NO:287), TFFRLFNRSFTQALGEGGGKNLRIIRKGIHIIKKY* (SEQ ID NO:288), TFFRLFSRSFTQALGKGGGKNLRIIRKGIHIIKKY* (SEQ ID NO:289), TFFRLFNRSFTQALGAGGGKNLRIIRKGIHIIKKY* (SEQ ID NO:290), TFFRLFDRSFTQALGKGGGKNLRIIRKGIHIIKKY* (SEQ ID NO:291), TFFRLFNRSFTQALGFGGGKNLRIIRKGIHIIKKY* (SEQ ID NO:292), TFFRAFARSFTQAAAKGGGKNLRIIRKGIHIIKKY* (SEQ ID NO:293), TFFRLFARSFTQAAGKGGGKNLRIIRKGIHIIKKY* (SEQ ID NO:294), TFFRLFNRSFTQLKGGGKNLRIIRKGIHIIKKY* (SEQ ID NO:295), TFFRLFNRSFTQALGSGGGKNLRIIRKGIHIIKKY* (SEQ ID NO:296), TLFRLFNRSFTQALGKGGGKNLRIIRKGIHIIKKY* (SEQ ID NO:297), TFFRLNFRSFTQALGKGGGKILRNIRKGIHIIKKY* (SEQ ID NO:298), TFFRLFNRSQTQALGKGGGKNLRIIRKGIHIIKKY* (SEQ ID NO:299), TFFRLFAAAFTQALGKGGGKNLRIIRKGIHIIKKY* (SEQ ID NO:300), TFFRLFNRSFTQALGKPYPKNLRIIRKGIHIIKKY* (SEQ ID NO:301), TFFRLFNRSAAAALGKGGGKNLRIIRKGIHIIKKY* (SEQ ID NO:302), TFFRLFFRSNTQALGKGGGKILRIIRKGIHINKKY* (SEQ ID NO:303), TFFRLFNRSFTQPLGKGGGKNLRIIRKGIHIIKKY* (SEQ ID NO:304), TAFRLANRSATQALGKGGGKNLRIIRKGIHIIKKY* (SEQ ID NO:305), TFFRLFNRSFTQAAAAGGGKNLRIIRKGIHIIKKY* (SEQ ID NO:306), TFFRLQNRSFTQALGKGGGKNLRIIRKGIHIIKKY* (SEQ ID NO:307), TFFRLFNRSFTQALPKGGGKNLRIIRKGIHIIKKY* (SEQ ID NO:308), TYYRLFNRSFTQALGKGGGKNLRIIRKGIHIIKKY* (SEQ ID NO:309), and TFFRLFRSFTQALGKGGGKNLRIIRKGIHIIKKY* (SEQ ID NO:310) where the asterisk indicates an optional amidated carboxyl terminus. Of course, this protecting group can be can be eliminated and/or substituted with another protecting group as described herein.

Without being bound by a particular theory, it was discovered that addition of a protecting group, particularly to the carboxyl and in certain embodiments the amino terminus can improve the stability and efficacy of the peptide.

A wide number of protecting groups are suitable for this purpose. Such groups include, but are not limited to acetyl, amide, and alkyl groups with acetyl and alkyl groups being particularly preferred for N-terminal protection and amide groups being preferred for carboxyl terminal protection. In certain particularly preferred embodiments, the protecting groups include, but are not limited to alkyl chains as in fatty acids, propionyl, formyl, and others. Particularly preferred carboxyl protecting groups include amides, esters, and ether-forming protecting groups. In one preferred embodiment, an acetyl group is used to protect the amino terminus and an amide group is used to protect the carboxyl terminus. These blocking groups enhance the helix-forming tendencies of the peptides. Certain particularly preferred blocking groups include alkyl groups of various lengths, e.g., groups having the formula: CH₃—(CH₂)ₙ—CO— where n ranges from about 1 to about 20, preferably from about 1 to about 16 or 18, more preferably from about 3 to about 13, and most preferably from about 3 to about 10.

In certain embodiments, the protecting groups include, but are not limited to alkyl chains as in fatty acids, propionyl, formyl, and others. Particularly preferred carboxyl protecting groups include amides, esters, and ether-forming protecting groups. In one embodiment, an acetyl group is used to protect the amino terminus and/or an amino group is used to protect the carboxyl terminus (i.e., amidated carboxyl terminus). In certain embodiments blocking groups include alkyl groups of various lengths, e.g., groups having the formula: CH₃—(CH₂)ₙ—CO— where n ranges from about 3 to about 20, preferably from about 3 to about 16, more preferably from about 3 to about 13, and most preferably from about 3 to about 10.

In certain embodiments, the acid group on the C-terminal can be blocked with an alcohol, aldehyde or ketone group and/or the N-terminal residue can have the natural amide group, or be blocked with an acyl, carboxylic acid, alcohol, aldehyde, or ketone group.

Other protecting groups include, but are not limited to Fmoc, t-butoxycarbonyl (t-BOC), 9-fluoreneacetyl group, 1-fluorenecarboxylic group, 9-florenecarboxylic group, 9-fluorenone-1-carboxylic group, benzyloxycarbonyl, xanthyl (Xan), trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), benzyloxy (BzlO), benzyl (Bzl), benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl—Z), 2-bromobenzyloxycarbonyl (2-Br—Z), Benzyloxymethyl (Bom), cyclohexyloxy (cHxO), t-butoxymethyl (Bum), t-butoxy (tBuO), t-Butyl (tBu), Acetyl (Ac), and Trifluoroacetyl (TFA).

Protecting/blocking groups are well known to those of skill as are methods of coupling such groups to the appropriate residue(s) comprising the peptides of this invention (see, e.g., Greene et al., (1991) Protective Groups in Organic Synthesis, 2nd ed., John Wiley & Sons, Inc. Somerset, N.J.). In illustrative embodiment, for example, acetylation is accomplished during the synthesis when the peptide is on the resin using acetic anhydride. Amide protection can be achieved by the selection of a proper resin for the synthesis. For example, a rink amide resin can be used. After the completion of the synthesis, the semipermanent protecting groups on acidic bifunctional amino acids such as Asp and Glu and basic amino acid Lys, hydroxyl of Tyr are all simultaneously removed. The peptides released from such a resin using acidic treatment comes out with the n-terminal protected as acetyl and the carboxyl protected as NH₂ and with the simultaneous removal of all of the other protecting groups.

Where amino acid sequences are disclosed herein, amino acid sequences comprising, one or more protecting groups, e.g., as described above (or any other commercially available protecting groups for amino acids used, e.g., in boc or fmoc peptide synthesis) are also contemplated.

### Peptide Circularization.

In certain embodiments the peptides described herein are circularized/cyclized to produce cyclic peptides. Cyclic peptides, as contemplated herein, include head/tail, head/side chain, tail/side chain, and side chain/side chain cyclized peptides. In addition, peptides contemplated herein include homodet, containing only peptide bonds, and heterodet containing in addition disulfide, ester, thioester-bonds, or other bonds.

The cyclic peptides can be prepared using virtually any art-known technique for the preparation of cyclic peptides. For example, the peptides can be prepared in linear or non-cyclized form using conventional solution or solid phase peptide syntheses and cyclized using standard chemistries. Preferably, the chemistry used to cyclize the peptide will be sufficiently mild so as to avoid substantially degrading the peptide. Suitable procedures for synthesizing the peptides described herein as well as suitable chemistries for cyclizing the peptides are well known in the art.

In various embodiments cyclization can be achieved via direct coupling of the N- and C-terminus to form a peptide (or other) bond, but can also occur via the amino acid side chains. Furthermore it can be based on the use of other functional groups, including but not limited to amino, hydroxy, sulfhydryl, halogen, sulfonyl, carboxy, and thiocarboxy. These groups can be located at the amino acid side chains or be attached to their N- or C-terminus.

Accordingly, it is to be understood that the chemical linkage used to covalently cyclize the peptides of the invention need not be an amide linkage. In many instances it may be desirable to modify the N- and C-termini of the linear or non-cyclized peptide so as to provide, for example, reactive groups that may be cyclized under mild reaction conditions. Such linkages include, by way of example and not limitation amide, ester, thioester, CH₂—NH, etc. Techniques and reagents for synthesizing peptides having modified termini and chemistries suitable for cyclizing such modified peptides are well-known in the art.

Alternatively, in instances where the ends of the peptide are conformationally or otherwise constrained so as to make cyclization difficult, it may be desirable to attach linkers to the N- and/or C-termini to facilitate peptide cyclization. Of course, it will be appreciated that such linkers will bear reactive groups capable of forming covalent bonds with the termini of the peptide. Suitable linkers and chemistries are well-known in the art and include those previously described.

Cyclic peptides and depsipeptides (heterodetic peptides that include ester (depside) bonds as part of their backbone) have been well characterized and show a wide spectrum of biological activity. The reduction in conformational freedom brought about by cyclization often results in higher receptor-binding affinities. Frequently in these cyclic compounds, extra conformational restrictions are also built in, such as the use of D- and N-alkylated-amino acids, α,β-dehydro amino acids or α,α-disubstituted amino acid residues.

Methods of forming disulfide linkages in peptides are well known to those of skill in the art (see, e.g., Eichler and Houghten (1997) Protein Pept. Lett. 4: 157-164).

Reference may also be made to Marlowe (1993) Biorg. Med. Chem. Lett. 3: 437-44 who describes peptide cyclization on TFA resin using trimethylsilyl (TMSE) ester as an orthogonal protecting group; Pallin and Tam (1995) J. Chem. Soc. Chem. Comm. 2021-2022) who describe the cyclization of unprotected peptides in aqueous solution by oxime formation; Algin et al. (1994) Tetrahedron Lett. 35: 9633-9636 who disclose solid-phase synthesis of head-to-tail cyclic peptides via lysine side-chain anchoring; Kates et al. (1993) Tetrahedron Lett. 34: 1549-1552 who describe the production of head-to-tail cyclic peptides by three-dimensional solid phase strategy; Tumelty et al. (1994) J. Chem. Soc. Chem. Comm. 1067-1068, who describe the synthesis of cyclic peptides from an immobilized activated intermediate, where activation of the immobilized peptide is carried out with N-protecting group intact and subsequent removal leading to cyclization; McMurray et al. (1994) Peptide Res. 7: 195-206) who disclose head-to-tail cyclization of peptides attached to insoluble supports by means of the side chains of aspartic and glutamic acid; Hruby et al. (1994) Reactive Polymers 22: 231-241) who teach an alternate method for cyclizing peptides via solid supports; and Schmidt and Langer (1997) J. Peptide Res. 49: 67-73, who disclose a method for synthesizing cyclotetrapeptides and cyclopentapeptides.

These methods of peptide cyclization are illustrative and non-limiting. Using the teaching provide herein, other cyclization methods will be available to one of skill in the art.

### Identification/Verification of Active Peptides

The active AMPs, STAMPs and the like can be identified and/or validated using an in vitro screening assay. Indeed, in many instances the AMPs and/or STAMPS described herein will be used in vitro as preservatives, topical antimicrobial treatments, and the like. Additionally, despite certain apparent limitations of in vitro susceptibility tests, clinical data indicate that a good correlation exists between minimal inhibitory concentration (MIC) test results and in vivo efficacy of antibiotic compounds (see, e.g., Murray et al. (1994) Antimicrobial Susceptibility Testing, Poupard et al., eds., Plenum Press, New York; Knudsen et al. (1995) Antimicrob. Agents Chemother. 39(6): 1253-1258; and the like). Thus, AMPs useful for treating infections and diseases related thereto are also conveniently identified by demonstrated in vitro antimicrobial activity against specified microbial targets, e.g., as illustrated in Table 4).

Typically, the in vitro antimicrobial activity of antimicrobial agents is tested using standard NCCLS bacterial inhibition assays, or MIC tests (see, National Committee on Clinical Laboratory Standards “Performance Standards for Antimicrobial Susceptibility Testing,” NCCLS Document M100-S5 Vol. 14, No. 16, December 1994; “Methods for dilution antimicrobial susceptibility test for bacteria that grow aerobically-Third Edition,” Approved Standard M7-A3, National Committee for Clinical Standards, Villanova, Pa.).

It will be appreciated that other assays as are well known in the art or that will become apparent to those having skill in the art upon review of this disclosure may also be used to identify active AMPs. Such assays include, for example, the assay described in Lehrer et al. (1988) J. Immunol. Meth., 108: 153 and Steinberg and Lehrer, “Designer Assays for Antimicrobial Peptides: Disputing the ‘One Size Fits All’ Theory,” In: Antibacterial Peptide Protocols, Shafer, Ed., Humana Press, N.J. Generally, active peptides of the invention will exhibit MICs (as measured using the assays described in the examples) of less than about 100 μM, preferably less than about 80 or 60 μM, more preferably about 50 μM or less, about 25 μM or less, or about 15 μM or less, or about 10 μM or less.

### Administration and Formulations.

Pharmaceutical Formulations.

In certain embodiments, the constructs described herein (e.g., targeting peptides attached to antimicrobial peptide(s), targeting peptides attached to detectable label(s), etc.) are administered to a mammal in need thereof, to a cell, to a tissue, to a composition (e.g., a food), etc.). In various embodiments the compositions can be administered to detect and/or locate, and/or quantify the presence of particular microorganisms, microorganism populations, biofilms comprising particular microorganisms, and the like. In various embodiments the compositions can be administered to inhibit particular microorganisms, microorganism populations, biofilms comprising particular microorganisms, and the like.

These active agents (antimicrobial peptides and/or chimeric moieties) can be administered in the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Methods of formulating such derivatives are known to those of skill in the art. For example, the disulfide salts of a number of delivery agents are described in PCT Publication WO 2000/059863 which is incorporated herein by reference. Similarly, acid salts of therapeutic peptides, peptoids, or other mimetics, and can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the active agents herein include halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. In certain embodiments basic salts include alkali metal salts, e.g., the sodium salt, and copper salts.

For the preparation of salt forms of basic drugs, the pKa of the counterion is preferably at least about 2 pH lower than the pKa of the drug. Similarly, for the preparation of salt forms of acidic drugs, the pKa of the counterion is preferably at least about 2 pH higher than the pKa of the drug. This permits the counterion to bring the solution's pH to a level lower than the pHmax to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (i.e., break down into the individual entities of drug and counterion) in an aqueous environment.

Preferably, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

In various embodiments preparation of esters typically involves functionalization of hydroxyl and/or carboxyl groups that are present within the molecular structure of the active agent. In certain embodiments, the esters are typically acyl-substituted derivatives of free alcohol groups, i.e., moieties that are derived from carboxylic acids of the formula RCOOH where R is alkyl, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the active agents identified herein are useful for parenteral, topical, oral, nasal (or otherwise inhaled), rectal, or local administration, such as by aerosol or transdermally, for detection and/or quantification, and or localization, and/or prophylactic and/or therapeutic treatment of infection (e.g., microbial infection). The compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, lipid complexes, etc.

The active agents (e.g., antimicrobial peptides and/or chimeric constructs) described herein can also be combined with a pharmaceutically acceptable carrier (excipient) to form a pharmacological composition. In certain embodiments, pharmaceutically acceptable carriers include those approved by a regulatory agency of the Federal or a state government or listed in the U.S. Pharmacopeia or other generally recognized pharmacopeia for use in/on animals, and more particularly in/on humans. A “carrier” refers to, for example, a diluent, adjuvant, excipient, auxiliary agent or vehicle with which an active agent of the present invention is administered.

Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds, particularly of use in the preparation of tablets, capsules, gel caps, and the like include, but are not limited to binders, diluent/fillers, disentegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g. calcium carbonate, carboxymethylcellulose calcium, sodium starch glycollate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., active peptide) and the resulting composition is compressed. Where necessary the compressed product is coated, e.g., known methods for masking the taste or for enteric dissolution or sustained release. Suitable coating materials include, but are not limited to ethyl-cellulose, hydroxymethylcellulose, polyoxyethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain therapeutic applications, the compositions of this invention are administered, e.g., topically administered or administered to the oral or nasal cavity, to a patient suffering from infection or at risk for infection or prophylactically to prevent dental caries or other pathologies of the teeth or oral mucosa characterized by microbial infection in an amount sufficient to prevent and/or cure and/or at least partially prevent or arrest the disease and/or its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms in) the patient.

The concentration of active agent(s) can vary widely, and will be selected primarily based on activity of the active ingredient(s), body weight and the like in accordance with the particular mode of administration selected and the patient's needs. Concentrations, however, will typically be selected to provide dosages ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 50 mg/kg/day. In certain embodiments, dosages range from about 20 mg to about 50 mg given orally twice daily. It will be appreciated that such dosages may be varied to optimize a therapeutic and/or phophylactic regimen in a particular subject or group of subjects.

In certain embodiments, the active agents of this invention are administered to the oral cavity. This is readily accomplished by the use of lozenges, aerosol sprays, mouthwash, coated swabs, and the like.

In certain embodiments, the active agent(s) of this invention are administered topically, e.g., to the skin surface, to a topical lesion or wound, to a surgical site, and the like.

In certain embodiments the active agents of this invention are administered systemically (e.g., orally, or as an injectable) in accordance with standard methods well known to those of skill in the art. In other preferred embodiments, the agents, can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Other formulations for topical delivery include, but are not limited to, ointments, gels, sprays, fluids, and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

As indicated above, various buccal, and sublingual formulations are also contemplated.

In certain embodiments, one or more active agents of the present invention can be provided as a “concentrate”, e.g., in a storage container (e.g., in a premeasured volume) ready for dilution, or in a soluble capsule ready for addition to a volume of water, alcohol, hydrogen peroxide, or other diluent.

While the invention is described with respect to use in humans, it is also suitable for animal, e.g., veterinary use. Thus certain preferred organisms include, but are not limited to humans, non-human primates, canines, equines, felines, porcines, ungulates, largomorphs, and the like.

Nanoemulsion Formulations.

In certain embodiments peptides and/or chimeric moieties (e.g., STAMPs) as described herein are formulated in a nanoemulsion. Nanoemulsions include, but are not limited to oil in water (O/W) nanoemulsions, and water in oil (W/O) nanoemulsions. Nanoemulsions can be defined as emulsions with mean droplet diameters ranging from about 20 to about 1000 nm. Usually, the average droplet size is between about 20 nm or 50 nm and about 500 nm. The terms sub-micron emulsion (SME) and mini-emulsion are used as synonyms.

Illustrative oil in water (O/W) nanoemulsions include, but are not limited to:

Surfactant micelles—micelles composed of small molecules surfactants or detergents (e.g., SDS/PBS/2-propanol) which are suitable for predominantly hydrophobic peptides.

Polymer micelles—micelles composed of polymer, copolymer, or block copolymer surfactants (e.g., Pluronic L64/PBS/2-propanol) which are suitable for predominantly hydrophobic peptides;

Blended micelles: micelles in which there is more than one surfactant component or in which one of the liquid phases (generally an alcohol or fatty acid compound) participates in the formation of the micelle (e.g., Octanoic acid/PBS/EtOH) which are suitable for predominantly hydrophobic peptides;

Integral peptide micelles—blended micelles in which the peptide serves as an auxiliary surfactant, forming an integral part of the micelle (e.g., amphipathic peptide/PBS/mineral oil) which are suitable for amphipathic peptides; and

Pickering (solid phase) emulsions—emulsions in which the peptides are associated with the exterior of a solid nanoparticle (e.g., polystyrene nanoparticles/PBS/no oil phase) which are suitable for amphipathic peptides.

Illustrative water in oil (W/O) nanoemulsions include, but are not limited to:

Surfactant micelles—micelles composed of small molecules surfactants or detergents (e.g., dioctyl sulfosuccinate/PBS/2-propanol, Isopropylmyristate/PBS/2-propanol, etc.) which are suitable for predominantly hydrophilic peptides;

Polymer micelles—micelles composed of polymer, copolymer, or block copolymer surfactants (e.g., PLURONIC® L121/PBS/2-propanol), which are suitable for predominantly hydrophilic peptides;

Blended micelles—micelles in which there is more than one surfactant component or in which one of the liquid phases (generally an alcohol or fatty acid compound) participates in the formation of the micelle (e.g., capric/caprylic diglyceride/PBS/EtOH) which are suitable for predominantly hydrophilic peptides;

Integral peptide micelles—blended micelles in which the peptide serves as an auxiliary surfactant, forming an integral part of the micelle (e.g., amphipathic peptide/PBS/polypropylene glycol) which are suitable for amphipathic peptides; and

Pickering (solid phase) emulsions—emulsions in which the peptides are associated with the exterior of a solid nanoparticle (e.g., chitosan nanoparticles/no aqueous phase/mineral oil) which are suitable for amphipathic peptides.

As indicated above, in certain embodiments the nanoemulsions comprise one or more surfactants or detergents. In some embodiments the surfactant is a non-anionic detergent (e.g., a polysorbate surfactant, a polyoxyethylene ether, etc.). Surfactants that find use in the present invention include, but are not limited to surfactants such as the TWEEN®, TRITON®, and TYLOXAPOL® families of compounds.

In certain embodiments the emulsions further comprise one or more cationic halogen containing compounds, including but not limited to, cetylpyridinium chloride. In still further embodiments, the compositions further comprise one or more compounds that increase the interaction (“interaction enhancers”) of the composition with microorganisms (e.g., chelating agents like ethylenediaminetetraacetic acid, or ethylenebis(oxyethylenenitrilo)tetraacetic acid in a buffer).

In some embodiments, the nanoemulsion further comprises an emulsifying agent to aid in the formation of the emulsion. Emulsifying agents include compounds that aggregate at the oil/water interface to form a kind of continuous membrane that prevents direct contact between two adjacent droplets. Certain embodiments of the present invention feature oil-in-water emulsion compositions that may readily be diluted with water to a desired concentration without impairing their anti-pathogenic properties.

In addition to discrete oil droplets dispersed in an aqueous phase, certain oil-in-water emulsions can also contain other lipid structures, such as small lipid vesicles (e.g., lipid spheres that often consist of several substantially concentric lipid bilayers separated from each other by layers of aqueous phase), micelles (e.g., amphiphilic molecules in small clusters of 50-200 molecules arranged so that the polar head groups face outward toward the aqueous phase and the apolar tails are sequestered inward away from the aqueous phase), or lamellar phases (lipid dispersions in which each particle consists of parallel amphiphilic bilayers separated by thin films of water).

These lipid structures are formed as a result of hydrophobic forces that drive apolar residues (e.g., long hydrocarbon chains) away from water. The above lipid preparations can generally be described as surfactant lipid preparations (SLPs). SLPs are minimally toxic to mucous membranes and are believed to be metabolized within the small intestine (see e.g., Hamouda et al., (1998) J. Infect. Disease 180: 1939).

In certain embodiments the emulsion comprises a discontinuous oil phase distributed in an aqueous phase, a first component comprising an alcohol and/or glycerol, and a second component comprising a surfactant or a halogen-containing compound. The aqueous phase can comprise any type of aqueous phase including, but not limited to, water (e.g., dionized water, distilled water, tap water) and solutions (e.g., phosphate buffered saline solution, or other buffer systems). The oil phase can comprise any type of oil including, but not limited to, plant oils (e.g., soybean oil, avocado oil, flaxseed oil, coconut oil, cottonseed oil, squalene oil, olive oil, canola oil, corn oil, rapeseed oil, safflower oil, and sunflower oil), animal oils (e.g., fish oil), flavor oil, water insoluble vitamins, mineral oil, and motor oil. In certain embodiments, the oil phase comprises 30-90 vol % of the oil-in-water emulsion (i.e., constitutes 30-90% of the total volume of the final emulsion), more preferably 50-80%.

In certain embodiments the alcohol, when present, is ethanol.

While the present invention is not limited by the nature of the surfactant, in some preferred embodiments, the surfactant is a polysorbate surfactant (e.g., TWEEN 20®, TWEEN 40®, TWEEN 60®, and TWEEN 80®), a pheoxypolyethoxyethanol (e.g., TRITON® X-100, X-301, X-165, X-102, and X-200, and TYLOXAPOL®), or sodium dodecyl sulfate, and the like.

In certain embodiments a halogen-containing component is present. the nature of the halogen-containing compound, in some preferred embodiments the halogen-containing compound comprises a chloride salt (e.g., NaCl, KCl, etc.), a cetylpyridinium halide, a cetyltrimethylammonium halide, a cetyldimethylethylammonium halide, a cetyldimethylbenzylammonium halide, a cetyltributylphosphonium halide, dodecyltrimethylammonium halides, tetradecyltrimethylammonium halides, cetylpyridinium chloride, cetyltrimethylammonium chloride, cetylbenzyldimethylammonium chloride, cetylpyridinium bromide, cetyltrimethylammonium bromide, cetyldimethylethylammonium bromide, cetyltributylphosphonium bromide, dodecyltrimethylammonium bromide, tetradecyltrimethylammonium bromide, and the like

In certain embodiments the emulsion comprises a quaternary ammonium compound. Quaternary ammonium compounds include, but are not limited to, N-alkyldimethyl benzyl ammonium saccharinate, 1,3,5-Triazine-1,3,5(2H,4H,6H)-triethanol; 1-Decanaminium, N-decyl-N,N-dimethyl-, chloride (or) Didecyl dimethyl ammonium chloride; 2-(2-(p-(Diisobutyl)cresosxy)ethoxy)ethyl dimethyl benzyl ammonium chloride; 2-(2-(p-(Diisobutyl)phenoxy)ethoxy)ethyl dimethyl benzyl ammonium chloride; alkyl 1 or 3 benzyl-1-(2-hydroxethyl)-2-imidazolinium chloride; alkyl bis(2-hydroxyethyl)benzyl ammonium chloride; alkyl demethyl benzyl ammonium chloride; alkyl dimethyl 3,4-dichlorobenzyl ammonium chloride (100% C12); alkyl dimethyl 3,4-dichlorobenzyl ammonium chloride (50% C14, 40% C12, 10% C16); alkyl dimethyl 3,4-dichlorobenzyl ammonium chloride (55% C14, 23% C12, 20% C16); alkyl dimethyl benzyl ammonium chloride; alkyl dimethyl benzyl ammonium chloride (100% C14); alkyl dimethyl benzyl ammonium chloride (100% C16); alkyl dimethyl benzyl ammonium chloride (41% C14, 28% C12); alkyl dimethyl benzyl ammonium chloride (47% C12, 18% C14); alkyl dimethyl benzyl ammonium chloride (55% C16, 20% C14); alkyl dimethyl benzyl ammonium chloride (58% C14, 28% C16); alkyl dimethyl benzyl ammonium chloride (60% C14, 25% C12); alkyl dimethyl benzyl ammonium chloride (61% C11, 23% C14); alkyl dimethyl benzyl ammonium chloride (61% C12, 23% C14); alkyl dimethyl benzyl ammonium chloride (65% C12, 25% C14); alkyl dimethyl benzyl ammonium chloride (67% C12, 24% C14); alkyl dimethyl benzyl ammonium chloride (67% C12, 25% C14); alkyl dimethyl benzyl ammonium chloride (90% C14, 5% C12); alkyl dimethyl benzyl ammonium chloride (93% C14, 4% C12); alkyl dimethyl benzyl ammonium chloride (95% C16, 5% C18); alkyl dimethyl benzyl ammonium chloride (and) didecyl dimethyl ammonium chloride; alkyl dimethyl benzyl ammonium chloride (as in fatty acids); alkyl dimethyl benzyl ammonium chloride (C12-C16); alkyl dimethyl benzyl ammonium chloride (C12-C18); alkyl dimethyl benzyl and dialkyl dimethyl ammonium chloride; alkyl dimethyl dimethylbenzyl ammonium chloride; alkyl dimethyl ethyl ammonium bromide (90% C14, 5% C16, 5% C12); alkyl dimethyl ethyl ammonium bromide (mixed alkyl and alkenyl groups as in the fatty acids of soybean oil); alkyl dimethyl ethylbenzyl ammonium chloride; alkyl dimethyl ethylbenzyl ammonium chloride (60% C14); alkyl dimethyl isopropylbenzyl ammonium chloride (50% C12, 30% C14, 17% C16, 3% C18); alkyl trimethyl ammonium chloride (58% C18, 40% C16, 1% C14, 1% C12); alkyl trimethyl ammonium chloride (90% C18, 10% C16); alkyldimethyl(ethylbenzyl) ammonium chloride (C12-18); Di-(C8-10)-alkyl dimethyl ammonium chlorides; dialkyl dimethyl ammonium chloride; dialkyl dimethyl ammonium chloride; dialkyl dimethyl ammonium chloride; dialkyl methyl benzyl ammonium chloride; didecyl dimethyl ammonium chloride; diisodecyl dimethyl ammonium chloride; dioctyl dimethyl ammonium chloride; dodecyl bis(2-hydroxyethyl) octyl hydrogen ammonium chloride; dodecyl dimethyl benzyl ammonium chloride; dodecylcarbamoyl methyl dimethyl benzyl ammonium chloride; heptadecyl hydroxyethylimidazolinium chloride; hexahydro-1,3,5-tris(2-hydroxyethyl)-s-triazine; myristalkonium chloride (and) Quat RNIUM 14; N,N-Dimethyl-2-hydroxypropylammonium chloride polymer; n-alkyl dimethyl benzyl ammonium chloride; n-alkyl dimethyl ethylbenzyl ammonium chloride; n-tetradecyl dimethyl benzyl ammonium chloride monohydrate; octyl decyl dimethyl ammonium chloride; octyl dodecyl dimethyl ammonium chloride; octyphenoxyethoxyethyl dimethyl benzyl ammonium chloride; oxydiethylenebis (alkyl dimethyl ammonium chloride); quaternary ammonium compounds, dicoco alkyldimethyl, chloride; trimethoxysilyl propyl dimethyl octadecyl ammonium chloride; trimethoxysilyl quats, trimethyl dodecylbenzyl ammonium chloride; n-dodecyl dimethyl ethylbenzyl ammonium chloride; n-hexadecyl dimethyl benzyl ammonium chloride; n-tetradecyl dimethyl benzyl ammonium chloride; n-tetradecyl dimethyl ethylbenzyl ammonium chloride; and n-octadecyl dimethyl benzyl ammonium chloride.

Nanoemulsion formulations and methods of making such are well known to those of skill in the art and described for example in U.S. Pat. Nos. 7,476,393, 7,468,402, 7,314,624, 6,998,426, 6,902,737, 6,689,371, 6,541,018, 6,464,990, 6,461,625, 6,419,946, 6,413,527, 6,375,960, 6,335,022, 6,274,150, 6,120,778, 6,039,936, 5,925,341, 5,753,241, 5,698,219, an d5,152,923 and in Fanun et al. (2009) Microemulsions: Properties and Applications (Surfactant Science), CRC Press, Boca Raton Fla.

Formulations Optimizing Activity.

In certain embodiments, formulations are selected to optimize binding specificity, and/or binding avidity, and/or antimicrobial activity, and/or stability/conformation of the targeting peptide, antimicrobial peptide, chimeric moiety, and/or STAMP. In this regard, it was a surprising discovery that the activity of certain STAMPs, and presumably the constituent targeting peptides and/or antimicrobial peptides was optimized in the presence of a salt. Accordingly, certain embodiments are contemplated where the targeting peptide and/or antimicrobial peptide, and/or STAMP is formulated in combination with one or more salts. The formulations disclosed herein, however, are not limited to those containing salt(s). Embodiments, are also contemplated where the targeting peptide and/or antimicrobial peptide, and/or STAMP is formulated without the presence of a salt.

In certain embodiments, sodium chloride plus a little potassium chloride resulted in the best activity of the salts tested. However, other salts, e.g., CaCl₂, MgCl₂, MnCl₂ also enhanced activity. Accordingly, in certain embodiments, it is contemplated that the targeting peptide(s), and/or antimicrobial peptide(s), and/or chimeric moieties, and/or STAMPs are formulated with one or more salts.

In certain embodiments suitable salts include any of a number of pharmaceutically acceptable salts. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, besylate, glucoheptonate, lactobionate, and laurylsulphonate salts and the like (see, e.g., Berge et al. (1977) J. Pharm. Sci. 66: 1-19).

In certain embodiments pharmaceutically acceptable salts of the present invention include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, benzenesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately treating the compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra; and Stahl and Wermuth (2002) Handbook of Pharmaceutical Salts: Properties, Selection, and Use, Wiley-VCH, Zurich, Switzerland).

In various embodiments, the salt is simply a sodium chloride and/or a potassium chloride and can readily be prepared, for example, as a phosphate buffered saline (PBS) solution. In certain embodiments, the salt concentration is comparable to that found in 0.5×PBS to about 2.5×PBS, more preferably from about 0.5×PBS to about 1.5×PBS. In certain embodiments optimum activity has been observed in 1×PBS.

In various embodiments, the pH of the formulation ranges from about pH 5.0 to about pH 8.5, preferably from about pH 6.0 to about pH 8.0, more preferably from about pH 7.0 to about pH 8.0. In certain embodiments the pH is about pH 7.4.

While optimum results have been observed for certain STAMPs using a PBS buffer system, other buffer systems are also acceptable. Such buffers include, but are not limited to sulfate buffers, carbonate buffers, Tris buffers, CHAPS buffers, PIPES buffers, and the like, as long as the salt is included.

In various embodiments, the targeting peptide, and/or antimicrobial peptide, and/or chimeric moiety, and/or STAMP is present in the formulation at a concentration ranging from about 1 nM, to about 1, 10, or 100 mM, more preferably from about 1 nM, about 10 nM, about 100 nM, about 1 μM, or about 10 μM to about 50 μM, about 100 μM, about 200 μm, about 300 μM, about 400 μM, or about 500 μM, preferably from about 1 μM, about 10 μM, about 25 μM, or about 50 μM to about 1 mM, about 10 mM, about 20 mM, or about 5 mM, most preferably from about 10 μM, about 20 μM, or about 50 μM to about 100 μM, about 150 μM, or about 200 μm.

Home Health Care/Hygiene Product Formulations.

In certain embodiments, one or more of the targeting peptide(s), and/or antimicrobial peptides (AMPs) and/or chimeric moieties, and/or STAMPS described herein are incorporated into healthcare formulations, e.g., for home use. Such formulations include, but are not limited to toothpaste, mouthwash, tooth whitening strips or solutions, contact lens storage, wetting, or cleaning solutions, dental floss, toothpicks, toothbrush bristles, oral sprays, oral lozenges, nasal sprays, aerosolizers for oral and/or nasal application, wound dressings (e.g., bandages), and the like.

For example, chimeric moieties and/or STAMPs, and/or AMPs directed against S. mutans are well suited for inhibiting frequency or severity of dental caries formation, plaque formation, periodontal disease, and/or halitosis.

Chimeric moieties and/or STAMPs, and/or AMPs directed against Corynebacterium spp, when applied to a skin surface can reduce/eliminate Corynebacterium resulting in a reduction of odors. Such moieties are readily incorporated in soaps, antibiotics, antiseptics, disinfectants, and the like.

The formulation of such health products is well known to those of skill, and the antimicrobial peptides and/or chimeric constructs are simply added to such formulations in an effective dose (e.g., a prophylactic dose to inhibit dental care formation, etc.).

For example, toothpaste formulations are well known to those of skill in the art. Typically such formulations are mixtures of abrasives and surfactants; anticaries agents, such as fluoride; tartar control ingredients, such as tetrasodium pyrophosphate and methyl vinyl ether/maleic anhydride copolymer; pH buffers; humectants, to prevent dry-out and increase the pleasant mouth feel; and binders, to provide consistency and shape (see, e.g., Table 6). Binders keep the solid phase properly suspended in the liquid phase to prevent separation of the liquid phase out of the toothpaste. They also provide body to the dentifrice, especially after extrusion from the tube onto the toothbrush.

TABLE 6
Typical components of toothpaste.

| Ingredients | Wt % |
|---|---|
| Humectants | 40-70 |
| Water | 0-50 |
| Buffers/salts/tartar | 0.5-10 |
| control | |
| Organic thickeners | 0.4-2 |
| (gums) | |
| Inorganic thickeners | 0-12 |
| Abrasives | 10-50 |
| Actives (e.g., triclosan) | 0.2-1.5 |
| Surfactants | 0.5-2 |
| Flavor and sweetener | 0.8-1.5 |

Fluoride sources provide 468-15000 ppm fluorine.

Table 7 lists typical ingredients used in formulations; the final combination will depend on factors such as ingredient compatibility and cost, local customs, and desired benefits and quality to be delivered in the product. It will be recognized that one or more antimicrobial peptides and/or chimeric constructs described herein can simply be added to such formulations or used in place of one or more of the other ingredients.

TABLE 7
List of typical ingredients.

| | Inorganic | | | | Tartar Control |
|---|---|---|---|---|---|
| Gums | Thickeners | Abrasives | Surfactants | Humectants | Ingredient |
| Sodium | Silica | Hydrated | Sodium lauryl | Glycerine | Tetrasodium |
| carboxymethyl | thickeners | silica | sulfate | | pyrophosphate |
| cellulose | | | | | |
| Cellulose | Sodium | Dicalcium | Sodium N- | Sorbitol | Gantrez S-70 |
| ethers | aluminum | phosphate | lauryl | | |
| | silicates | dihydrate | sarcosinate | | |
| Xanthan Gum | Clays | Calcium | Pluronics | Propylene | Sodium tri- |
| | | carbonate | | glycol | polyphosphate |
| Carrageenans | | Sodium | | Xylitol | |
| | | bicarbonate | | | |
| Sodium | | Calcium | Sodium lauryl | Polyethylene | |
| alginate | | pyrophosphate | sulfoacetate | glycol | |
| Carbopols | | Alumina | | | |

One illustrative formulation described in U.S. Pat. No. 6,113,887 comprises (1) a water-soluble bactericide selected from the group consisting of pyridinium compounds, quaternary ammonium compounds and biguanide compounds in an amount of 0.001% to 5.0% by weight, based on the total weight of the composition; (2) a cationically-modified hydroxyethylcellulose having an average molecular weight of 1,000,000 or higher in the hydroxyethylcellulose portion thereof and having a cationization degree of 0.05 to 0.5 mol/glucose in an amount of 0.5% to 5.0% by weight, based on the total weight of the composition; (3) a surfactant selected from the group consisting of polyoxyethylene polyoxypropylene block copolymers and alkylolamide compounds in an amount of 0.5% to 13% by weight, based on the total weight of the composition; and (4) a polishing agent of the non-silica type in an amount of 5% to 50% by weight, based on the total weight of the composition. In certain embodiments, the antimicrobial peptide(s) and/or chimeric construct(s) described herein can be used in place of the bactericide or in combination with the bactericide.

Similarly, mouthwash formulations are also well known to those of skill in the art. Thus, for example, mouthwashes containing sodium fluoride are disclosed in U.S. Pat. Nos. 2,913,373, 3,975,514, and 4,548,809, and in US Patent Publications US 2003/0124068 A1, US 2007/0154410 A1, and the like. Mouthwashes containing various alkali metal compounds are also known: sodium benzoate (WO 9409752); alkali metal hypohalite (US 20020114851A1); chlorine dioxide (CN 1222345); alkali metal phosphate (US 2001/0002252 A1, US 2003/0007937 A1); hydrogen sulfate/carbonate (JP 8113519); cetylpyridium chloride (CPC) (see, e.g., U.S. Pat. No. 6,117,417, U.S. Pat. No. 5,948,390, and JP 2004051511). Mouthwashes containing higher alcohol (see, e.g., US 2002/0064505 A1, US 2003/0175216 A1); hydrogen peroxide (see, e.g., CN 1385145); CO₂ gas bubbles (see, e.g., JP 1275521 and JP 2157215) are also known. In certain embodiments, these and other mouthwash formulations can further comprise one or more of the AMPs or compound AMPs of this invention.

Contact lens storage, wetting, or cleaning solutions, dental floss, toothpicks, toothbrush bristles, oral sprays, oral lozenges, nasal sprays, and aerosolizers for oral and/or nasal application, and the like are also well known to those of skill in the art and can readily be adapted to incorporate one or more antimicrobial peptide(s) and/or chimeric construct(s) described herein.

The foregoing pharmaceutical and/or home healthcare formulations and/or devices are meant to be illustrative and not limiting. Using teaching provided herein, the antimicrobial peptide(s) and/or chimeric construct(s) described herein can readily be incorporated into other products.

Illustrative Oral Care Formulations.

The targeting peptide(s), and/or chimeric moieties, and/or STAMPs described herein can be used for a number of applications, e.g., as described above. In certain embodiments anti-S. mutans STAMPs, AMPs, and/or other chimeric moieties can be used to reduce the incidence or severity of dental caries, inhibit plaque formation, reduce halitosis, and the like. Accordingly, in certain embodiments, such moieties are included in devices and formulations for dental applications e.g., tea or other drinks, toothpick coatings, dental floss coatings, toothpaste, gel, mouthwash, varnish, even professional dental products.

In certain embodiments, methods of treating or reducing the incidence, duration, or severity of periodontal disease are provided. The methods can include applying to the gingival crevice or periodontal pocket a composition comprising a targeting peptide, and/or antimicrobial peptide, and/or STAMP, and/or other chimeric moiety as described herein with a carrier/stabilizing agent. In the composition applied, the carrier/stabilizing agent can provide retention, tissue penetration, deposition and sustained release of the active agent (e.g., STAMP) for reducing the population of specific bacterial species within a periodontal biofilm and associated tissues. In certain embodiments, the carrier agent provides penetration and retention into the gingival crevice or periodontal pocket and associated tissues with sustained release of the active agent to enhance the reduction in population of select bacteria within the gingival tissue and dentinal tubule tissue.

In various embodiments, carrier agents can include, but are not limited to polylactide, polyglycolide, polylactide-co-glycolide, polycaprolactone, cellulosic-based polymers, ethylene glycol polymers and its copolymers, oxyethylene polymers, polyvinyl alcohol, chitosan and hyaluronan and its copolymers. In an aspect, the carrier agents include hydroxyethyl cellulose, hydroxypropyl cellulose, hydroxypropylmethyl cellulose, hydroxymethyl cellulose, polyvinyl alcohol, polyethylene glycol, polyethylene oxide, ethylene oxide-propylene oxide co-polymers, chitosan, hyaluronan and its copolymers, or combinations thereof. In another aspect, the carrier agents include hyaluronan or hyaluronic acid and copolymers including salts of hyaluronic acid, esters of hyaluronic acid, cross-linked gels of hyaluronic acid, enzymatic derivatives of hyaluronic acid, chemically modified derivatives of hyaluronic acid or combinations thereof. As used herein, hyaluronic acid broadly refers to naturally occurring, microbial and synthetic derivatives of acidic polysaccharides of various molecular weights constituted by residues of D-glucuronic acid polysaccharides and N-acetyl-D-glucosamine.

In certain embodiments, the active agent (e.g., STAMP, AMP, etc.) and the carrier agent are in the form of an admixture, in the form of a complex, covalently coupled, or a combination thereof. In certain embodiments, the carrier agent comprises a bioadhesive. Suitable bioadhesive carrier agents include, but are not limited to a cellulose based polymer and/or a dextrin. Suitable cellulose based polymers include, but are not limited to hydroxyethyl cellulose, hydroxypropyl cellulose, hydroxymethyl cellulose, or a mixture thereof. In one illustrative embodiment, the bioadhesive carrier agent includes polylactide, polyglycolide, polylactide-co-glycolide, polyethylene glycol, hyaluronan, hyaluronic acid, chitosan, or a mixture thereof. In certain embodiments the bioadhesive carrier agent can include a copolymer comprising polyethylene glycol, hyaluronan, hyaluronic acid, chitosan, or a mixture thereof.

In certain embodiments, the carrier agent penetrates periodontal tissues. Suitable penetrating carrier agents include, but are not limited to hyaluronic acid, a hyaluronic acid derivative, chitosan, a chitosan derivative, or a mixture thereof. In an embodiment, the penetrating carrier agent includes a salt of hyaluronic acid, an ester of hyaluronic acid, an enzymatic derivative of hyaluronic acid, a cross-linked gel of hyaluronic acid, a chemically modified derivative of hyaluronic acid, or a mixture thereof.

### Microorganism Detection.

As indicated above, the targeting peptides and/or STAMPs are useful in diagnostic compositions and methods to determine the presence or absence and/or to quantify the amount of one or microorganisms present in the environment, in a food stuff, in a biological sample, and the like.

For example, targeting peptide-antimicrobial peptide conjugates (e.g. Specifically targeted antimicrobial peptides (STAMPs)) can be used as diagnostic reagents. STAMPs (and other targeted antimicrobial constructs described herein) have the ability to specifically bind to microorganisms, for example, S. mutans, and permeabilize or disrupt their membrane such that cell impermeable dyes or other reagent (propidium iodide, etc.) may enter the microorganism or intracellular molecules or contents (ATP, DNA, Calcium, etc.) of the targeted microorganism are caused to be released into the environment for analysis. In one method a STAMP, for example, C16G2, can permeabilize or disrupt the membrane of target microorganisms, for example, S. mutans, in a prepared culture or clinical sample by itself, in a biofilm in vitro or in vivo. To the sample a cell impermeable dye (e.g. propidium iodide, etc.) is added to label and allow for detection of those microorganisms targeted by the STAMP. Cell permeable dyes (e.g. SYTO9) can also be added to label and detect the entire population of microorganisms in the sample. Labeled cells can then be quantified by fluorescence microscopy, fluorometry, flow cytometry or other method.

In another example, a STAMP treated sample is mixed with luciferase and luciferin which reacts with the ATP released from the STAMP treated cells and the resulting luminescence is used to detected and quantify targeted cells.

### Kits.

In another embodiment kits are provided for the inhibition of an infection and/or for the treatment and/or prevention of dental caries in a mammal. The kits typically comprise a container containing one or more of the active agents (i.e., the antimicrobial peptide(s) and/or chimeric construct(s)) described herein. In certain embodiments the active agent(s) can be provided in a unit dosage formulation (e.g., suppository, tablet, caplet, patch, etc.) and/or may be optionally combined with one or more pharmaceutically acceptable excipients.

In certain embodiments the kits comprise one or more of the home healthcare product formulations described herein (e.g., toothpaste, mouthwash, tooth whitening strips or solutions, contact lens storage, wetting, or cleaning solutions, dental floss, toothpicks, toothbrush bristles, oral sprays, oral lozenges, nasal sprays, aerosolizers for oral and/or nasal application, and the like).

In certain embodiments kits are provided for detecting and/or locating and/or quantifying certain target microorganisms and/or cells or tissues comprising certain target microorganisms, and/or prosthesis bearing certain target microorganisms, and/or biofilms comprising certain target microorganisms. In various embodiments these kits typically comprise a chimeric moiety comprising a targeting peptide and a detectable label as described herein and/or a targeting peptide attached to an affinity tag for use in a pretargeting strategy as described herein.

In addition, the kits optionally include labeling and/or instructional materials providing directions (i.e., protocols) for the practice of the methods or use of the “therapeutics” or “prophylactics” or detection reagents of this invention. Certain instructional materials describe the use of one or more active agent(s) of this invention to therapeutically or prophylactically to inhibit or prevent infection and/or to inhibit the formation of dental caries. The instructional materials may also, optionally, teach preferred dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A targeting peptide that binds to Streptococcus mutans, where said peptide comprises or consists of the amino acid sequence
X¹-X²-F-R-X⁵-X⁶-X⁷-R-X⁹-X¹⁰-X¹¹-X¹²-X¹³-X¹⁴-X¹⁵-X¹⁶
or the inverse of said amino acid sequence, wherein:
X¹ is a polar amino acid, or A;
X² is F, W, Q, A, or an analog thereof;
X⁵ is a hydrophobic amino acid;
X⁶ is a hydrophobic amino acid, N, Q, or an analog thereof;
X⁷ is a polar amino acid, A, F, or an analog thereof;
X⁹ is a polar amino acid, A or an analog thereof;
X¹⁰ is a hydrophobic amino acid, Q, A, or an analog thereof;
X¹¹ is a hydrophobic amino acid;
X¹² is Q, A, or an analog thereof;
X¹³ is a non-polar amino acid;
X¹⁴ is a hydrophobic amino acid;
X¹⁵ is a non-polar amino acid, N, S, D, or an analog thereof;
X¹⁶ is a polar amino acid, F, A, or an analog thereof; and
said peptide ranges in length up to 100 amino acids.

2. The peptide of claim 1, wherein:
X¹ is A or T;
X² is F, W, Q, or A;
X⁵ is L, or A;
X⁶ is F, L, N, A, or Q;
X⁷ is A, or F;
X⁹ is S or A;
X¹⁰ is Q, or A;
X¹¹ is T, or A;
X¹² is Q or A;
X¹³ is P, or A;
X¹⁴ is L, or A;
X¹⁵ N, S, or D; and
X¹⁶ is K, or Q.

3. (canceled)

4. The peptide of claim 2, wherein X² is F.

5. (canceled)

6. The peptide of claim 2, wherein X⁵ is L.

7-8. (canceled)

9. The peptide of claim 3, wherein X⁶ is F.

10-13. (canceled)

14. The peptide of claim 2, wherein X⁷ is N.

15-16. (canceled)

17. The peptide of claim 2, wherein X⁹ is S.

18-20. (canceled)

21. The peptide of claim 2, wherein X¹⁰ is F.

22. (canceled)

23. The peptide of claim 2, wherein X¹¹ is T.

24. (canceled)

25. The peptide of claim 2, wherein X¹² is Q.

26. (canceled)

27. The peptide of claim 2, wherein X¹³ is A.

28. (canceled)

29. The peptide of claim 2, wherein X¹⁴ is L.

30-31. (canceled)

32. The peptide of claim 2, wherein X¹⁵ is G, or A.

33-35. (canceled)

36. The peptide of claim 2, wherein X¹⁶ is K.

37. The peptide of claim 1, wherein said peptide does not comprise the amino acid sequence TFFRLFNRSFTQALGK.

38. The peptide of claim 1, wherein said peptide comprises or consists of an amino acid sequence selected from the group consisting of AFFRAFNRAFAQALAK (SEQ ID NO:5), TFFRAFARAFAQAAAK (SEQ ID NO:6), AFFRAFARAFAQALAK (SEQ ID NO:7), AFFRLFARAFAQAAAK (SEQ ID NO:8), TLFRLLNRSLTQALGK (SEQ ID NO:9), TFFRLFNRSFTQALFK (SEQ ID NO:10), TFFRLFNRSLTQALGK (SEQ ID NO:11), TFFRLFNRSFTQALNK (SEQ ID NO:12), AFFRAFARAFAQAAAK (SEQ ID NO:13), AFFRAFNRAFAQAAAK (SEQ ID NO:14), TFFRLFNRSFTQALSK (SEQ ID NO:15), AFFRAFARSFAQAAAK (SEQ ID NO:16), AFFRAFARAFAQAAGK (SEQ ID NO:17), AFFRAFARAFTQAAAK (SEQ ID NO:18), TFFRLFNRSFTQALGQ (SEQ ID NO:19), TFFRLLNRSFTQALGK (SEQ ID NO:20), TWFRLFNRSFTQALGK (SEQ ID NO:21), AFFRAFARAFAQAFAK (SEQ ID NO:22), TQFRLFNRSFTQALGK (SEQ ID NO:23), TFFRLFNRSFTQALDK (SEQ ID NO:24), TFFRLFNRSFTQALAK (SEQ ID NO:25), TFFRLFNRSFTQALGE (SEQ ID NO:26), TFFRLFSRSFTQALGK (SEQ ID NO:27), TFFRLFNRSFTQALGA (SEQ ID NO:28), TFFRLFDRSFTQALGK (SEQ ID NO:29), TFFRLFNRSFTQALGF (SEQ ID NO:30), TFFRAFARSFTQAAAK (SEQ ID NO:31), TFFRLFARSFTQAAGK (SEQ ID NO:32), TFFRLFNRSFTQ L K (SEQ ID NO:33), TFFRLFNRSFTQALGS (SEQ ID NO:34), TLFRLFNRSFTQALGK (SEQ ID NO:35), TFFRLNFRSFTQALGK (SEQ ID NO:36), TFFRLFNRSQTQALGK (SEQ ID NO:37), TFFRLFAAAFTQALGK (SEQ ID NO:38), TFFRLFNRSFTQALGK (SEQ ID NO:39), TFFRLFNRSAAAALGK (SEQ ID NO:40), TFFRLFFRSNTQALGK (SEQ ID NO:41), TFFRLFNRSFTQPLGK (SEQ ID NO:42), TAFRLANRSATQALGK (SEQ ID NO:43), TFFRLFNRSFTQAAAA (SEQ ID NO:44), TFFRLQNRSFTQALGK (SEQ ID NO:45), TFFRLFNRSFTQALPK (SEQ ID NO:46), TYYRLFNRSFTQALGK (SEQ ID NO:47), TFFRLF RSFTQALGK (SEQ ID NO:48), and TQFRLQNRSQTQALGK (SEQ ID NO:49).

39-44. (canceled)

45. The of claim 1, wherein said peptide is attached to an effector moiety selected from the group consisting of a detectable label, a porphyrin or other photosensitizer, an antimicrobial peptide, an antibiotic, a ligand, a lipid or liposome, an agent that physically disrupts the extracellular matrix within a community of microorganisms, and a polymeric particle.

46. The peptide of claim 45, wherein said peptide is attached to an antimicrobial peptide.

47. (canceled)

48. The peptide of claim 46, wherein said peptide is attached to an antimicrobial peptide comprising or consisting of an amino acid sequence selected from the group consisting of G2 KNLRIIRKGIHIIKKY* (SEQ ID NO:2), Novispirin G10 KNLRRIIRKGIHIIKKYG (SEQ ID NO:49), Novispirin T10 KNLRRIIRKTIHIIKKYG (SEQ ID NO:50), Novispirin G7 KNLRRIGRKIIHIIKKYG (SEQ ID NO:51), Novispirin T7 KNLRRITRKIIHIIKKYG (SEQ ID NO:52), Ovispirin KNLRRIIRKIIHIIKKYG (SEQ ID NO:53), PGG GLLRRLRKKIGEIFKKYG (SEQ ID NO:54), Protegrin-1 RGGRLCYCRRRFCVCVGR* (SEQ ID NO:55), K-1 GLGRVIGRLIKQIIWRR (SEQ ID NO:56), K-2 VYRKRKSILKIYAKLKGWH (SEQ ID NO:57), K-7 NYRLVNAIFSKIFKKKFIKF (SEQ ID NO:58), K-8 KILKFLFKKVF (SEQ ID NO:59), K-9 FIRKFLKKWLL (SEQ ID NO:60), K-10 KLFKFLRKHLL (SEQ ID NO:61), K-11 KILKFLFKQVF (SEQ ID NO:62), K-12 KILKKLFKFVF (SEQ ID NO:63), K-13 GILKKLFTKVF (SEQ ID NO:64), K-14 LRKFLHKLF (SEQ ID NO:65), K-15 LRKNLRWLF (SEQ ID NO:66), K-16 FIRKFLQKLHL (SEQ ID NO:67), K-17 FTRKFLKFLHL (SEQ ID NO:68), K-18 KKFKKFKVLKIL (SEQ ID NO:69), K-19 LLKLLKLKKLKF (SEQ ID NO:70), K-20 FLKFLKKFFKKLKY (SEQ ID NO:71), K-21 GWLKMFKKIIGKFGKF (SEQ ID NO:72), K-22 GIFKKFVKILYKVQKL (SEQ ID NO:73), and B-33 FKKFWKWFRRF (SEQ ID NO:107).

49-66. (canceled)

67. An antimicrobial peptide that ranges in length up to 100 amino acids, where said peptide comprises or consists of an amino acid sequence selected from the group consisting of FIGAIARLLSKIFGKR (SEQ ID NO:228), GIFSKLAGKKIKNLLISG (SEQ ID NO:229), GIFSKLAGKKIKNLLISGLKG (SEQ ID NO:230), GLFSKFVGKGIKNFLIKGVK (SEQ ID NO:231), KAYSTPRCKGLFRALMCWL (SEQ ID NO:232), KIFGAIWPLALGALKNLIK (SEQ ID NO:233), GWGSFFKKAAHVGKHVGKAALTHYL (SEQ ID NO:234), RGLRRLGRKIAHGVKKYG (SEQ ID NO:235), RGLRRLGRKIAHGVKKYGPTVLRIIRIAG (SEQ ID NO:236), KIAHGVKKYGPTVLRIIR (SEQ ID NO:237), LLGDFFRKSKEKIGKEFKRIVQRIKDFLRNLVPRTES (SEQ ID NO:238), FLPLIGRVLSGIL (SEQ ID NO:239), IGKFLKKAKKFGKAFVKILKK (SEQ ID NO:240), GKFLKKAKKFGKAFVKIL (SEQ ID NO:241), WFLKFLKKFFKKLKY (SEQ ID NO:242), RGLRRLGRKIAHGVKKY (SEQ ID NO:243), LLGDFFRKSKEKI (SEQ ID NO:244), and ILRWPWWPWRRK (SEQ ID NO:245).

68-74. (canceled)

75. A pharmaceutical composition comprising a peptide of claim 1 attached to an antimicrobial peptide in a pharmaceutically acceptable carrier.

76-77. (canceled)

78. A method of killing or inhibiting the growth or proliferation of a bacterium, said method comprising:
contacting said bacterium or a biofilm comprising said bacterium with a composition comprising a targeting peptide of claim 1 attached to an antimicrobial peptide, and/or to an antibiotic, and/or to a porphyrin or other photosensitizer.

79. A method of reducing or preventing the formation of dental caries and/or the incidence or severity of periodontal disease in a mammal, said method comprising:
administering to the oral cavity of said mammal a composition comprising a targeting peptide of claim 1 attached to an antimicrobial peptide, and/or to an antibiotic, and/or to a porphyrin or other photosensitizer.

80-88. (canceled)

89. A method of detecting a bacterium and/or a bacterial film, said method comprising:
contacting said bacterium or bacterial film with a composition comprising a targeting peptide of claim 1 attached to a detectable label; and
detecting said detectable label wherein the quantity and/or location of said detectable label is an indicator of the presence of said bacterium and/or bacterial film.

90. (canceled)

91. A composition comprising a targeting peptide of claim 1 attached to a photosensitizing agent.

92-95. (canceled)

96. A method of inhibiting the growth or proliferation of a microorganism or a biofilm, said method comprising contacting said microorganism or biofilm with a composition comprising a targeting peptide of claim 1 attached to a photosensitizing agent.

97. A method of reducing or preventing the formation of dental caries and/or the incidence or severity of periodontal disease in a mammal, said method comprising:
administering to the oral cavity of said mammal a composition comprising a targeting peptide of claim 1 attached to a photosensitizing agent.

98-102. (canceled)


SEQUENCE LISTING (310 sequences)

SEQ ID NO: 1 - protein, 16 aa, Artificial
  Chemically synthesized peptide
XXFRXXXRXXXXXXXX

SEQ ID NO: 2 - protein, 16 aa, Artificial
  Chemically synthesized peptide
TFFRLFNRSFTQALGK

SEQ ID NO: 3 - protein, 16 aa, Artificial
  Chemically synthesized peptide
KNLRIIRKGIHIIKKY

SEQ ID NO: 4 - protein, 35 aa, Artificial
  Chemically synthesized peptide
TFFRLFNRSFTQALGKGGGKNLRIIRKGIHIIKKY

SEQ ID NO: 5 - protein, 16 aa, Artificial
  Chemically synthesized peptide
AFFRAFNRAFAQALAK

SEQ ID NO: 6 - protein, 16 aa, Artificial
  Chemically synthesized peptide
TFFRAFARAFAQAAAK

SEQ ID NO: 7 - protein, 16 aa, Artificial
  Chemically synthesized peptide
AFFRAFARAFAQALAK

SEQ ID NO: 8 - protein, 16 aa, Artificial
  Chemically synthesized peptide
AFFRLFARAFAQAAAK

SEQ ID NO: 9 - protein, 16 aa, Artificial
  Chemically synthesized peptide
TLFRLLNRSLTQALGK

SEQ ID NO: 10 - protein, 16 aa, Artificial
  Chemically synthesized peptide
TFFRLFNRSFTQALFK

SEQ ID NO: 11 - protein, 16 aa, Artificial
  Chemically synthesized peptide
TFFRLFNRSLTQALGK

SEQ ID NO: 12 - protein, 16 aa, Artificial
  Chemically synthesized peptide
TFFRLFNRSFTQALNK

SEQ ID NO: 13 - protein, 16 aa, Artificial
  Chemically synthesized peptide
AFFRAFARAFAQAAAK

SEQ ID NO: 14 - protein, 16 aa, Artificial
  Chemically synthesized peptide
AFFRAFNRAFAQAAAK

SEQ ID NO: 15 - protein, 16 aa, Artificial
  Chemically synthesized peptide
TFFRLFNRSFTQALSK

SEQ ID NO: 16 - protein, 16 aa, Artificial
  Chemically synthesized peptide
AFFRAFARSFAQAAAK

SEQ ID NO: 17 - protein, 16 aa, Artificial
  Chemically synthesized peptide
AFFRAFARAFAQAAGK

SEQ ID NO: 18 - protein, 16 aa, Artificial
  Chemically synthesized peptide
AFFRAFARAFTQAAAK

SEQ ID NO: 19 - protein, 16 aa, Artificial
  Chemically synthesized peptide
TFFRLFNRSFTQALGQ

SEQ ID NO: 20 - protein, 16 aa, Artificial
  Chemically synthesized peptide
TFFRLLNRSFTQALGK

SEQ ID NO: 21 - protein, 16 aa, Artificial
  Chemically synthesized peptide
TWFRLFNRSFTQALGK

SEQ ID NO: 22 - protein, 16 aa, Artificial
  Chemically synthesized peptide
AFFRAFARAFAQAFAK

SEQ ID NO: 23 - protein, 16 aa, Artificial
  Chemically synthesized peptide
TQFRLFNRSFTQALGK

SEQ ID NO: 24 - protein, 16 aa, Artificial
  Chemically synthesized peptide
TFFRLFNRSFTQALDK

SEQ ID NO: 25 - protein, 16 aa, Artificial
  Chemically synthesized peptide
TFFRLFNRSFTQALAK

SEQ ID NO: 26 - protein, 16 aa, Artificial
  Chemically synthesized peptide
TFFRLFNRSFTQALGE

SEQ ID NO: 27 - protein, 16 aa, Artificial
  Chemically synthesized peptide
TFFRLFSRSFTQALGK

SEQ ID NO: 28 - protein, 16 aa, Artificial
  Chemically synthesized peptide
TFFRLFNRSFTQALGA

SEQ ID NO: 29 - protein, 16 aa, Artificial
  Chemically synthesized peptide
TFFRLFDRSFTQALGK

SEQ ID NO: 30 - protein, 16 aa, Artificial
  Chemically synthesized peptide
TFFRLFNRSFTQALGF

SEQ ID NO: 31 - protein, 16 aa, Artificial
  Chemically synthesized peptide
TFFRAFARSFTQAAAK

SEQ ID NO: 32 - protein, 16 aa, Artificial
  Chemically synthesized peptide
TFFRLFARSFTQAAGK

SEQ ID NO: 33 - protein, 14 aa, Artificial
  Chemically synthesized peptide
TFFRLFNRSFTQLK

SEQ ID NO: 34 - protein, 16 aa, Artificial
  Chemically synthesized peptide
TFFRLFNRSFTQALGS

SEQ ID NO: 35 - protein, 16 aa, Artificial
  Chemically synthesized peptide
TLFRLFNRSFTQALGK

SEQ ID NO: 36 - protein, 16 aa, Artificial
  Chemically synthesized peptide
TFFRLNFRSFTQALGK

SEQ ID NO: 37 - protein, 16 aa, Artificial
  Chemically synthesized peptide
TFFRLFNRSQTQALGK

SEQ ID NO: 38 - protein, 16 aa, Artificial
  Chemically synthesized peptide
TFFRLFAAAFTQALGK

SEQ ID NO: 39 - protein, 16 aa, Artificial
  Chemically synthesized peptide
TFFRLFNRSFTQALGK

SEQ ID NO: 40 - protein, 16 aa, Artificial
  Chemically synthesized peptide
TFFRLFNRSAAAALGK

SEQ ID NO: 41 - protein, 16 aa, Artificial
  Chemically synthesized peptide
TFFRLFFRSNTQALGK

SEQ ID NO: 42 - protein, 16 aa, Artificial
  Chemically synthesized peptide
TFFRLFNRSFTQPLGK

SEQ ID NO: 43 - protein, 16 aa, Artificial
  Chemically synthesized peptide
TAFRLANRSATQALGK

SEQ ID NO: 44 - protein, 16 aa, Artificial
  Chemically synthesized peptide
TFFRLFNRSFTQAAAA

SEQ ID NO: 45 - protein, 16 aa, Artificial
  Chemically synthesized peptide
TFFRLQNRSFTQALGK

SEQ ID NO: 46 - protein, 16 aa, Artificial
  Chemically synthesized peptide
TFFRLFNRSFTQALPK

SEQ ID NO: 47 - protein, 16 aa, Artificial
  Chemically synthesized peptide
TYYRLFNRSFTQALGK

SEQ ID NO: 48 - protein, 15 aa, Artificial
  Chemically synthesized peptide
TFFRLFRSFTQALGK

SEQ ID NO: 49 - protein, 16 aa, Artificial
  Chemically synthesized peptide
TQFRLQNRSQTQALGK

SEQ ID NO: 50 - protein, 18 aa, Artificial
  Chemically synthesized peptide
KNLRRIIRKGIHIIKKYG

SEQ ID NO: 51 - protein, 18 aa, Artificial
  Chemically synthesized peptide
KNLRRIIRKTIHIIKKYG

SEQ ID NO: 52 - protein, 18 aa, Artificial
  Chemically synthesized peptide
KNLRRIGRKIIHIIKKYG

SEQ ID NO: 53 - protein, 18 aa, Artificial
  Chemically synthesized peptide
KNLRRITRKIIHIIKKYG

SEQ ID NO: 54 - protein, 18 aa, Artificial
  Chemically synthesized peptide
KNLRRIIRKIIHIIKKYG

SEQ ID NO: 55 - protein, 18 aa, Artificial
  Chemically synthesized peptide
GLLRRLRKKIGEIFKKYG

SEQ ID NO: 56 - protein, 18 aa, Artificial
  Chemically synthesized peptide
RGGRLCYCRRRFCVCVGR

SEQ ID NO: 57 - protein, 17 aa, Artificial
  Chemically synthesized peptide
GLGRVIGRLIKQIIWRR

SEQ ID NO: 58 - protein, 19 aa, Artificial
  Chemically synthesized peptide
VYRKRKSILKIYAKLKGWH

SEQ ID NO: 59 - protein, 20 aa, Artificial
  Chemically synthesized peptide
NYRLVNAIFSKIFKKKFIKF

SEQ ID NO: 60 - protein, 11 aa, Artificial
  Chemically synthesized peptide
KILKFLFKKVF

SEQ ID NO: 61 - protein, 11 aa, Artificial
  Chemically synthesized peptide
FIRKFLKKWLL

SEQ ID NO: 62 - protein, 11 aa, Artificial
  Chemically synthesized peptide
KLFKFLRKHLL

SEQ ID NO: 63 - protein, 11 aa, Artificial
  Chemically synthesized peptide
KILKFLFKQVF

SEQ ID NO: 64 - protein, 11 aa, Artificial
  Chemically synthesized peptide
KILKKLFKFVF

SEQ ID NO: 65 - protein, 11 aa, Artificial
  Chemically synthesized peptide
GILKKLFTKVF

SEQ ID NO: 66 - protein, 9 aa, Artificial
  Chemically synthesized peptide
LRKFLHKLF

SEQ ID NO: 67 - protein, 9 aa, Artificial
  Chemically synthesized peptide
LRKNLRWLF

SEQ ID NO: 68 - protein, 11 aa, Artificial
  Chemically synthesized peptide
FIRKFLQKLHL

SEQ ID NO: 69 - protein, 11 aa, Artificial
  Chemically synthesized peptide
FTRKFLKFLHL

SEQ ID NO: 70 - protein, 12 aa, Artificial
  Chemically synthesized peptide
KKFKKFKVLKIL

SEQ ID NO: 71 - protein, 12 aa, Artificial
  Chemically synthesized peptide
LLKLLKLKKLKF

SEQ ID NO: 72 - protein, 14 aa, Artificial
  Chemically synthesized peptide
FLKFLKKFFKKLKY

SEQ ID NO: 73 - protein, 16 aa, Artificial
  Chemically synthesized peptide
GWLKMFKKIIGKFGKF

SEQ ID NO: 74 - protein, 16 aa, Artificial
  Chemically synthesized peptide
GIFKKFVKILYKVQKL

SEQ ID NO: 75 - protein, 24 aa, Artificial
  Chemically synthesized peptide
GRLVLEITADEVKALGEALANAKI

SEQ ID NO: 76 - protein, 21 aa, Artificial
  Chemically synthesized peptide
YIQFHLNQQPRPKVKKIKIFL

SEQ ID NO: 77 - protein, 32 aa, Artificial
  Chemically synthesized peptide
GSVIKKRRKRMAKKKHRKLLKKTRIQRRRAGK

SEQ ID NO: 78 - protein, 30 aa, Artificial
  Chemically synthesized peptide
MRFGSLALVAYDSAIKHSWPRPSSVRRLRM

SEQ ID NO: 79 - protein, 39 aa, Artificial
  Chemically synthesized peptide
FESKILNASKELDKEKKVNTALSFNSHQDFAKAYQNGKI

SEQ ID NO: 80 - protein, 18 aa, Artificial
  Chemically synthesized peptide
WSRVPGHSDTGWKVWHRW

SEQ ID NO: 81 - protein, 21 aa, Artificial
  Chemically synthesized peptide
MGIIAGIIKFIKGLIEKFTGK

SEQ ID NO: 82 - protein, 16 aa, Artificial
  Chemically synthesized peptide
RESKLIAMADMIRRRI

SEQ ID NO: 83 - protein, 24 aa, Artificial
  Chemically synthesized peptide
LSLATFAKIFMTRSNWSLKRFNRL

SEQ ID NO: 84 - protein, 30 aa, Artificial
  Chemically synthesized peptide
MIRIRSPTKKKLNRNSISDWKSNTSGRFFY

SEQ ID NO: 85 - protein, 37 aa, Artificial
  Chemically synthesized peptide
MKRRRCNWCGKLFYLEEKSKEAYCCKECRKKAKKVKK

SEQ ID NO: 86 - protein, 28 aa, Artificial
  Chemically synthesized peptide
VLPFPAIPLSRRRACVAAPRPRSRQRAS

SEQ ID NO: 87 - protein, 30 aa, Artificial
  Chemically synthesized peptide
KNKKQTDILEKVKEILDKKKKTKSVGQKLY

SEQ ID NO: 88 - protein, 15 aa, Artificial
  Chemically synthesized peptide
SLQSQLGPCLHDQRH

SEQ ID NO: 89 - protein, 28 aa, Artificial
  Chemically synthesized peptide
KFQGEFTNIGQSYIVSASHMSTSLNTGK

SEQ ID NO: 90 - protein, 41 aa, Artificial
  Chemically synthesized peptide
TKKIELKRFVDAFVKKSYENYILERELKKLIKAINEELPTK

SEQ ID NO: 91 - protein, 20 aa, Artificial
  Chemically synthesized peptide
KFSDQIDKGQDALKDKLGDL

SEQ ID NO: 92 - protein, 13 aa, Artificial
  Chemically synthesized peptide
LSEMERRRLRKRA

SEQ ID NO: 93 - protein, 25 aa, Artificial
  Chemically synthesized peptide
RRGCTERLRRMARRNAWDLYAEHFY

SEQ ID NO: 94 - protein, 26 aa, Artificial
  Chemically synthesized peptide
SKFKVLRKIIIKEYKGELMLSIQKQR

SEQ ID NO: 95 - protein, 20 aa, Artificial
  Chemically synthesized peptide
FELVDWLETNLGKILKSKSA

SEQ ID NO: 96 - protein, 22 aa, Artificial
  Chemically synthesized peptide
LVLRICTDLFTFIKWTIKQRKS

SEQ ID NO: 97 - protein, 24 aa, Artificial
  Chemically synthesized peptide
VYSFLYVLVIVRKLLSMKKRIERL

SEQ ID NO: 98 - protein, 18 aa, Artificial
  Chemically synthesized peptide
GIVLIGLKLIPLLANVLR

SEQ ID NO: 99 - protein, 21 aa, Artificial
  Chemically synthesized peptide
VMQSLYVKPPLILVTKLAQQN

SEQ ID NO: 100 - protein, 16 aa, Artificial
  Chemically synthesized peptide
SFMPEIQKNTIPTQMK

SEQ ID NO: 101 - protein, 39 aa, Artificial
  Chemically synthesized peptide
LGLTAGVAYAAQPTNQPTNQPTNQPTNQPTNQPTNQPRW

SEQ ID NO: 102 - protein, 15 aa, Artificial
  Chemically synthesized peptide
CGKLLEQKNFFLKTR

SEQ ID NO: 103 - protein, 30 aa, Artificial
  Chemically synthesized peptide
ASKQASKQASKQASKQASKQASRSLKNHLL

SEQ ID NO: 104 - protein, 24 aa, Artificial
  Chemically synthesized peptide
PDAPRTCYHKPILAALSRIVVTDR

SEQ ID NO: 105 - protein, 8 aa, Artificial
  Chemically synthesized peptide
NYAVVSHT

SEQ ID NO: 106 - protein, 23 aa, Artificial
  Chemically synthesized peptide
FQKPFTGEEVEDFQDDDEIPTII

SEQ ID NO: 107 - protein, 15 aa, Artificial
  Chemically synthesized peptide
GWRLIKKILRVFKGL

SEQ ID NO: 108 - protein, 11 aa, Artificial
  Chemically synthesized peptide
FKKFWKWFRRF

SEQ ID NO: 109 - protein, 11 aa, Artificial
  Chemically synthesized peptide
LKRFLKWFKRF

SEQ ID NO: 110 - protein, 11 aa, Artificial
  Chemically synthesized peptide
KLFKRWKHLFR

SEQ ID NO: 111 - protein, 11 aa, Artificial
  Chemically synthesized peptide
RLLKRFKHLFK

SEQ ID NO: 112 - protein, 11 aa, Artificial
  Chemically synthesized peptide
FKTFLKWLHRF

SEQ ID NO: 113 - protein, 11 aa, Artificial
  Chemically synthesized peptide
IKQLLHFFQRF

SEQ ID NO: 114 - protein, 11 aa, Artificial
  Chemically synthesized peptide
KLLQTFKQIFR

SEQ ID NO: 115 - protein, 11 aa, Artificial
  Chemically synthesized peptide
RILKELKNLFK

SEQ ID NO: 116 - protein, 11 aa, Artificial
  Chemically synthesized peptide
LKQFVHFIHRF

SEQ ID NO: 117 - protein, 11 aa, Artificial
  Chemically synthesized peptide
VKTLLHIFQRF

SEQ ID NO: 118 - protein, 11 aa, Artificial
  Chemically synthesized peptide
KLVEQLKEIFR

SEQ ID NO: 119 - protein, 11 aa, Artificial
  Chemically synthesized peptide
RVLQEIKQILK

SEQ ID NO: 120 - protein, 11 aa, Artificial
  Chemically synthesized peptide
VKNLAELVHRF

SEQ ID NO: 121 - protein, 11 aa, Artificial
  Chemically synthesized peptide
ATHLLHALQRF

SEQ ID NO: 122 - protein, 11 aa, Artificial
  Chemically synthesized peptide
KLAENVKEILR

SEQ ID NO: 123 - protein, 11 aa, Artificial
  Chemically synthesized peptide
RALHEAKEALK

SEQ ID NO: 124 - protein, 11 aa, Artificial
  Chemically synthesized peptide
FHYFWHWFHRF

SEQ ID NO: 125 - protein, 11 aa, Artificial
  Chemically synthesized peptide
LYHFLHWFQRF

SEQ ID NO: 126 - protein, 11 aa, Artificial
  Chemically synthesized peptide
YLFQTWQHLFR

SEQ ID NO: 127 - protein, 11 aa, Artificial
  Chemically synthesized peptide
YLLTEFQHLFK

SEQ ID NO: 128 - protein, 11 aa, Artificial
  Chemically synthesized peptide
FKTFLQWLHRF

SEQ ID NO: 129 - protein, 11 aa, Artificial
  Chemically synthesized peptide
IKTLLHFFQRF

SEQ ID NO: 130 - protein, 11 aa, Artificial
  Chemically synthesized peptide
KLLQTFNQIFR

SEQ ID NO: 131 - protein, 11 aa, Artificial
  Chemically synthesized peptide
TILQSLKNIFK

SEQ ID NO: 132 - protein, 11 aa, Artificial
  Chemically synthesized peptide
LKQFVKFIHRF

SEQ ID NO: 133 - protein, 11 aa, Artificial
  Chemically synthesized peptide
VKQLLKIFNRF

SEQ ID NO: 134 - protein, 11 aa, Artificial
  Chemically synthesized peptide
KLVQQLKNIFR

SEQ ID NO: 135 - protein, 11 aa, Artificial
  Chemically synthesized peptide
RVLNQVKQILK

SEQ ID NO: 136 - protein, 11 aa, Artificial
  Chemically synthesized peptide
VKKLAKLVRRF

SEQ ID NO: 137 - protein, 11 aa, Artificial
  Chemically synthesized peptide
AKRLLKVLKRF

SEQ ID NO: 138 - protein, 11 aa, Artificial
  Chemically synthesized peptide
KLAQKVKRVLR

SEQ ID NO: 139 - protein, 11 aa, Artificial
  Chemically synthesized peptide
RALKRIKHVLK

SEQ ID NO: 140 - protein, 7 aa, Artificial
  Chemically synthesized peptide
RRRRWWW

SEQ ID NO: 141 - protein, 7 aa, Artificial
  Chemically synthesized peptide
RRWWRRW

SEQ ID NO: 142 - protein, 7 aa, Artificial
  Chemically synthesized peptide
RRRWWWR

SEQ ID NO: 143 - protein, 7 aa, Artificial
  Chemically synthesized peptide
RWRWRWR

SEQ ID NO: 144 - protein, 7 aa, Artificial
  Chemically synthesized peptide
RRRFWWR

SEQ ID NO: 145 - protein, 7 aa, Artificial
  Chemically synthesized peptide
RRWWRRF

SEQ ID NO: 146 - protein, 7 aa, Artificial
  Chemically synthesized peptide
RRRWWWF

SEQ ID NO: 147 - protein, 7 aa, Artificial
  Chemically synthesized peptide
RWRWRWF

SEQ ID NO: 148 - protein, 7 aa, Artificial
  Chemically synthesized peptide
RRRRWWK

SEQ ID NO: 149 - protein, 7 aa, Artificial
  Chemically synthesized peptide
RRWWRRK

SEQ ID NO: 150 - protein, 7 aa, Artificial
  Chemically synthesized peptide
RRRWWWK

SEQ ID NO: 151 - protein, 7 aa, Artificial
  Chemically synthesized peptide
RWRWRWK

SEQ ID NO: 152 - protein, 7 aa, Artificial
  Chemically synthesized peptide
RRRKWWK

SEQ ID NO: 153 - protein, 7 aa, Artificial
  Chemically synthesized peptide
RRWKRRK

SEQ ID NO: 154 - protein, 7 aa, Artificial
  Chemically synthesized peptide
RRRKWWK

SEQ ID NO: 155 - protein, 7 aa, Artificial
  Chemically synthesized peptide
RWRKRWK

SEQ ID NO: 156 - protein, 14 aa, Artificial
  Chemically synthesized peptide
LHLLHQLLHLLHQF

SEQ ID NO: 157 - protein, 14 aa, Artificial
  Chemically synthesized peptide
AQAAHQAAHAAHQF

SEQ ID NO: 158 - protein, 14 aa, Artificial
  Chemically synthesized peptide
KLKKLLKKLKKLLK

SEQ ID NO: 159 - protein, 14 aa, Artificial
  Chemically synthesized peptide
LKLLKKLLKLLKKF

SEQ ID NO: 160 - protein, 14 aa, Artificial
  Chemically synthesized peptide
LQLLKQLLKLLKQF

SEQ ID NO: 161 - protein, 14 aa, Artificial
  Chemically synthesized peptide
AQAAKQAAKAAKQF

SEQ ID NO: 162 - protein, 14 aa, Artificial
  Chemically synthesized peptide
RWRRWWRHFHHFFH

SEQ ID NO: 163 - protein, 14 aa, Artificial
  Chemically synthesized peptide
KLKKLLKRWRRWWR

SEQ ID NO: 164 - protein, 14 aa, Artificial
  Chemically synthesized peptide
RWRRLLKKLHHLLH

SEQ ID NO: 165 - protein, 14 aa, Artificial
  Chemically synthesized peptide
KLKKLLKHLHHLLH

SEQ ID NO: 166 - protein, 15 aa, Artificial
  Chemically synthesized peptide
FVFRHKWVWKHRFLF

SEQ ID NO: 167 - protein, 15 aa, Artificial
  Chemically synthesized peptide
VFIHRHVWVHKHVLF

SEQ ID NO: 168 - protein, 15 aa, Artificial
  Chemically synthesized peptide
WRWRARWRWRLRWRF

SEQ ID NO: 169 - protein, 15 aa, Artificial
  Chemically synthesized peptide
WRIHLRARLHVKFRF

SEQ ID NO: 170 - protein, 15 aa, Artificial
  Chemically synthesized peptide
LRIHARFKVHIRLKF

SEQ ID NO: 171 - protein, 15 aa, Artificial
  Chemically synthesized peptide
FHIKFRVHLKVRFHF

SEQ ID NO: 172 - protein, 15 aa, Artificial
  Chemically synthesized peptide
FHVKIHFRLHVKFHF

SEQ ID NO: 173 - protein, 15 aa, Artificial
  Chemically synthesized peptide
LHIHAHFHVHIHLHF

SEQ ID NO: 174 - protein, 15 aa, Artificial
  Chemically synthesized peptide
FKIHFRLKVHIRFKF

SEQ ID NO: 175 - protein, 15 aa, Artificial
  Chemically synthesized peptide
FKAHIRFKLRVKFHF

SEQ ID NO: 176 - protein, 15 aa, Artificial
  Chemically synthesized peptide
LKAKIKFKVKLKIKF

SEQ ID NO: 177 - protein, 14 aa, Artificial
  Chemically synthesized peptide
WIWKHKFLHRHFLF

SEQ ID NO: 178 - protein, 14 aa, Artificial
  Chemically synthesized peptide
VFLHRHVIKHKLVF

SEQ ID NO: 179 - protein, 13 aa, Artificial
  Chemically synthesized peptide
FLHKHVLRHRIVF

SEQ ID NO: 180 - protein, 13 aa, Artificial
  Chemically synthesized peptide
VFKHKIVHRHILF

SEQ ID NO: 181 - protein, 13 aa, Artificial
  Chemically synthesized peptide
FLFKHLFLHRIFF

SEQ ID NO: 182 - protein, 12 aa, Artificial
  Chemically synthesized peptide
LFKHILIHRVIF

SEQ ID NO: 183 - protein, 12 aa, Artificial
  Chemically synthesized peptide
FLHKHLFKHKLF

SEQ ID NO: 184 - protein, 12 aa, Artificial
  Chemically synthesized peptide
VFRHRFIHRHVF

SEQ ID NO: 185 - protein, 12 aa, Artificial
  Chemically synthesized peptide
FIHKLVHKHVLF

SEQ ID NO: 186 - protein, 12 aa, Artificial
  Chemically synthesized peptide
VLRHLFRHRIVF

SEQ ID NO: 187 - protein, 12 aa, Artificial
  Chemically synthesized peptide
LVHKLILRHLLF

SEQ ID NO: 188 - protein, 12 aa, Artificial
  Chemically synthesized peptide
VFKRVLIHKLIF

SEQ ID NO: 189 - protein, 12 aa, Artificial
  Chemically synthesized peptide
IVRKFLFRHKVF

SEQ ID NO: 190 - protein, 12 aa, Artificial
  Chemically synthesized peptide
VLKHVIAHKRLF

SEQ ID NO: 191 - protein, 11 aa, Artificial
  Chemically synthesized peptide
FIRKFLFKHLF

SEQ ID NO: 192 - protein, 11 aa, Artificial
  Chemically synthesized peptide
VIRHVWVRKLF

SEQ ID NO: 193 - protein, 13 aa, Artificial
  Chemically synthesized peptide
FLFRHRFRHRLVF

SEQ ID NO: 194 - protein, 13 aa, Artificial
  Chemically synthesized peptide
LFLHKHAKHKFLF

SEQ ID NO: 195 - protein, 11 aa, Artificial
  Chemically synthesized peptide
FKHKFKHKFIF

SEQ ID NO: 196 - protein, 11 aa, Artificial
  Chemically synthesized peptide
LRHRLRHRLIF

SEQ ID NO: 197 - protein, 11 aa, Artificial
  Chemically synthesized peptide
LILKFLFKFVF

SEQ ID NO: 198 - protein, 11 aa, Artificial
  Chemically synthesized peptide
VLIRILVRVIF

SEQ ID NO: 199 - protein, 9 aa, Artificial
  Chemically synthesized peptide
FRHRFRHRF

SEQ ID NO: 200 - protein, 9 aa, Artificial
  Chemically synthesized peptide
LKHKLKHKF

SEQ ID NO: 201 - protein, 9 aa, Artificial
  Chemically synthesized peptide
FKFKHKLIF

SEQ ID NO: 202 - protein, 9 aa, Artificial
  Chemically synthesized peptide
LRLRHRVLF

SEQ ID NO: 203 - protein, 9 aa, Artificial
  Chemically synthesized peptide
FKFLFKFLF

SEQ ID NO: 204 - protein, 9 aa, Artificial
  Chemically synthesized peptide
LRLFLRWLF

SEQ ID NO: 205 - protein, 9 aa, Artificial
  Chemically synthesized peptide
FKFLFKHKF

SEQ ID NO: 206 - protein, 9 aa, Artificial
  Chemically synthesized peptide
LRLFLRHRF

SEQ ID NO: 207 - protein, 7 aa, Artificial
  Chemically synthesized peptide
FKFLFKF

SEQ ID NO: 208 - protein, 7 aa, Artificial
  Chemically synthesized peptide
LRLFLRF

SEQ ID NO: 209 - protein, 14 aa, Artificial
  Chemically synthesized peptide
HHFFHHFHHFFHHF

SEQ ID NO: 210 - protein, 14 aa, Artificial
  Chemically synthesized peptide
FHFFHHFFHFFHHF

SEQ ID NO: 211 - protein, 21 aa, Artificial
  Chemically synthesized peptide
KLLKGATFHFFHHFFHFFHHF

SEQ ID NO: 212 - protein, 18 aa, Artificial
  Chemically synthesized peptide
KLLKFHFFHHFFHFFHHF

SEQ ID NO: 213 - protein, 18 aa, Artificial
  Chemically synthesized peptide
FHFFHHFFHFFHHFKLLK

SEQ ID NO: 214 - protein, 7 aa, Artificial
  Chemically synthesized peptide
YSPWTNF

SEQ ID NO: 215 - protein, 37 aa, Artificial
  Chemically synthesized peptide
LLGDFFRKSKEKIGKEFKRIVQRIKDFLRNLVPRTES

SEQ ID NO: 216 - protein, 38 aa, Artificial
  Chemically synthesized peptide
CLLGDFFRKSKEKIGKEFKRIVQRIKDFLRNLVPRTES

SEQ ID NO: 217 - protein, 16 aa, Artificial
  Chemically synthesized peptide
FKRIVQRIKDFLRNLV

SEQ ID NO: 218 - protein, 39 aa, Artificial
  Chemically synthesized peptide
CLLGDFFRKSKEKIGKEFKRIVQRIKDFLRNLVPRTESC

SEQ ID NO: 219 - protein, 13 aa, Artificial
  Chemically synthesized peptide
FKRIVQRIKDFLR

SEQ ID NO: 220 - protein, 21 aa, Artificial
  Chemically synthesized peptide
FKRIVQRIKDFLRNLVPRTES

SEQ ID NO: 221 - protein, 21 aa, Artificial
  Chemically synthesized peptide
GKEFKRIVQRIKDFLRNLVPR

SEQ ID NO: 222 - protein, 20 aa, Artificial
  Chemically synthesized peptide
KRIVQRIKDFLRNLVPRTES

SEQ ID NO: 223 - protein, 21 aa, Artificial
  Chemically synthesized peptide
LLGDFFRKSKEKIGKEFKRIV

SEQ ID NO: 224 - protein, 31 aa, Artificial
  Chemically synthesized peptide
RKSKEKIGKEFKRIVQRIKDFLRNLVPRTES

SEQ ID NO: 225 - protein, 21 aa, Artificial
  Chemically synthesized peptide
SKEKIGKEFKRIVQRIKDFLR

SEQ ID NO: 226 - protein, 38 aa, Artificial
  Chemically synthesized peptide
LLGDFFRKSKEKIGKEFKRIVQRIKDFLRNLVPRTESC

SEQ ID NO: 227 - protein, 11 aa, Artificial
  Chemically synthesized peptide
KLFKFLRKHLL

SEQ ID NO: 228 - protein, 13 aa, Artificial
  Chemically synthesized peptide
FLKFLKKFFKKLK

SEQ ID NO: 229 - protein, 16 aa, Artificial
  Chemically synthesized peptide
FIGAIARLLSKIFGKR

SEQ ID NO: 230 - protein, 18 aa, Artificial
  Chemically synthesized peptide
GIFSKLAGKKIKNLLISG

SEQ ID NO: 231 - protein, 21 aa, Artificial
  Chemically synthesized peptide
GIFSKLAGKKIKNLLISGLKG

SEQ ID NO: 232 - protein, 20 aa, Artificial
  Chemically synthesized peptide
GLFSKFVGKGIKNFLIKGVK

SEQ ID NO: 233 - protein, 19 aa, Artificial
  Chemically synthesized peptide
KAYSTPRCKGLFRALMCWL

SEQ ID NO: 234 - protein, 19 aa, Artificial
  Chemically synthesized peptide
KIFGAIWPLALGALKNLIK

SEQ ID NO: 235 - protein, 25 aa, Artificial
  Chemically synthesized peptide
GWGSFFKKAAHVGKHVGKAALTHYL

SEQ ID NO: 236 - protein, 18 aa, Artificial
  Chemically synthesized peptide
RGLRRLGRKIAHGVKKYG

SEQ ID NO: 237 - protein, 29 aa, Artificial
  Chemically synthesized peptide
RGLRRLGRKIAHGVKKYGPTVLRIIRIAG

SEQ ID NO: 238 - protein, 18 aa, Artificial
  Chemically synthesized peptide
KIAHGVKKYGPTVLRIIR

SEQ ID NO: 239 - protein, 37 aa, Artificial; identical to SEQ ID NO: 215
  Chemically synthesized peptide

SEQ ID NO: 240 - protein, 13 aa, Artificial
  Chemically synthesized peptide
FLPLIGRVLSGIL

SEQ ID NO: 241 - protein, 21 aa, Artificial
  Chemically synthesized peptide
IGKFLKKAKKFGKAFVKILKK

SEQ ID NO: 242 - protein, 18 aa, Artificial
  Chemically synthesized peptide
GKFLKKAKKFGKAFVKIL

SEQ ID NO: 243 - protein, 15 aa, Artificial
  Chemically synthesized peptide
WFLKFLKKFFKKLKY

SEQ ID NO: 244 - protein, 17 aa, Artificial
  Chemically synthesized peptide
RGLRRLGRKIAHGVKKY

SEQ ID NO: 245 - protein, 13 aa, Artificial
  Chemically synthesized peptide
LLGDFFRKSKEKI

SEQ ID NO: 246 - protein, 12 aa, Artificial
  Chemically synthesized peptide
ILRWPWWPWRRK

SEQ ID NO: 247 - protein, 8 aa, Artificial
  Chemically synthesized peptide
DYKDDDDK

SEQ ID NO: 248 - protein, 4 aa, Artificial
  Chemically synthesized peptide
HHHH

SEQ ID NO: 249 - protein, 5 aa, Artificial
  Chemically synthesized peptide
HHHHH

SEQ ID NO: 250 - protein, 6 aa, Artificial
  Chemically synthesized peptide
HHHHHH

SEQ ID NO: 251 - protein, 5 aa, Artificial
  Chemically synthesized peptide
PSGSP

SEQ ID NO: 252 - protein, 5 aa, Artificial
  Chemically synthesized peptide
ASASA

SEQ ID NO: 253 - protein, 15 aa, Artificial
  Chemically synthesized peptide
GGGGSGGGGSGGGGS

SEQ ID NO: 254 - protein, 4 aa, Artificial
  Chemically synthesized peptide
GGGG

SEQ ID NO: 255 - protein, 5 aa, Artificial
  Chemically synthesized peptide
PSPSP

SEQ ID NO: 256 - protein, 5 aa, Artificial
  Chemically synthesized peptide
PSPSP

SEQ ID NO: 257 - protein, 4 aa, Artificial
  Chemically synthesized peptide
KKKK

SEQ ID NO: 258 - protein, 4 aa, Artificial
  Chemically synthesized peptide
RRRR

SEQ ID NO: 259 - protein, 5 aa, Artificial
  Chemically synthesized peptide
ASASA

SEQ ID NO: 260 - protein, 6 aa, Artificial
  Chemically synthesized peptide
GGSGGS

SEQ ID NO: 261 - protein, 5 aa, Artificial
  Chemically synthesized peptide
GGGGS

SEQ ID NO: 262 - protein, 10 aa, Artificial
  Chemically synthesized peptide
GGGGSGGGGS

SEQ ID NO: 263 - protein, 15 aa, Artificial
  Chemically synthesized peptide
GGGGSGGGGSGGGGS

SEQ ID NO: 264 - protein, 20 aa, Artificial
  Chemically synthesized peptide
GGGGSGGGGSGGGGSGGGGS

SEQ ID NO: 265 - protein, 25 aa, Artificial
  Chemically synthesized peptide
GGGGSGGGGSGGGGSGGGGSGGGGS

SEQ ID NO: 266 - protein, 30 aa, Artificial
  Chemically synthesized peptide
GGGGSGGGGSGGGGSGGGGSGGGGSGGGGS

SEQ ID NO: 267 - protein, 35 aa, Artificial
  Chemically synthesized peptide
AFFRAFNRAFAQALAKGGGKNLRIIRKGIHIIKKY

SEQ ID NO: 268 - protein, 35 aa, Artificial
  Chemically synthesized peptide
TFFRAFARAFAQAAAKGGGKNLRIIRKGIHIIKKY

SEQ ID NO: 269 - protein, 35 aa, Artificial
  Chemically synthesized peptide
AFFRAFARAFAQALAKGGGKNLRIIRKGIHIIKKY

SEQ ID NO: 270 - protein, 35 aa, Artificial
  Chemically synthesized peptide
AFFRLFARAFAQAAAKGGGKNLRIIRKGIHIIKKY

SEQ ID NO: 271 - protein, 35 aa, Artificial
  Chemically synthesized peptide
TLFRLLNRSLTQALGKGGGKNLRIIRKGIHIIKKY

SEQ ID NO: 272 - protein, 35 aa, Artificial
  Chemically synthesized peptide
TFFRLFNRSFTQALFKGGGKNLRIIRKGIHIIKKY

SEQ ID NO: 273 - protein, 35 aa, Artificial
  Chemically synthesized peptide
TFFRLFNRSLTQALGKGGGKNLRIIRKGIHIIKKY

SEQ ID NO: 274 - protein, 35 aa, Artificial
  Chemically synthesized peptide
TFFRLFNRSFTQALNKGGGKNLRIIRKGIHIIKKY

SEQ ID NO: 275 - protein, 35 aa, Artificial
  Chemically synthesized peptide
AFFRAFARAFAQAAAKGGGKNLRIIRKGIHIIKKY

SEQ ID NO: 276 - protein, 35 aa, Artificial
  Chemically synthesized peptide
AFFRAFNRAFAQAAAKGGGKNLRIIRKGIHIIKKY

SEQ ID NO: 277 - protein, 35 aa, Artificial
  Chemically synthesized peptide
TFFRLFNRSFTQALSKGGGKNLRIIRKGIHIIKKY

SEQ ID NO: 278 - protein, 35 aa, Artificial
  Chemically synthesized peptide
AFFRAFARSFAQAAAKGGGKNLRIIRKGIHIIKKY

SEQ ID NO: 279 - protein, 35 aa, Artificial
  Chemically synthesized peptide
AFFRAFARAFAQAAGKGGGKNLRIIRKGIHIIKKY

SEQ ID NO: 280 - protein, 35 aa, Artificial
  Chemically synthesized peptide
AFFRAFARAFTQAAAKGGGKNLRIIRKGIHIIKKY

SEQ ID NO: 281 - protein, 35 aa, Artificial
  Chemically synthesized peptide
TFFRLFNRSFTQALGQGGGKNLRIIRKGIHIIKKY

SEQ ID NO: 282 - protein, 35 aa, Artificial
  Chemically synthesized peptide
TFFRLLNRSFTQALGKGGGKNLRIIRKGIHIIKKY

SEQ ID NO: 283 - protein, 35 aa, Artificial
  Chemically synthesized peptide
TWFRLFNRSFTQALGKGGGKNLRIIRKGIHIIKKY

SEQ ID NO: 284 - protein, 35 aa, Artificial
  Chemically synthesized peptide
AFFRAFARAFAQAFAKGGGKNLRIIRKGIHIIKKY

SEQ ID NO: 285 - protein, 35 aa, Artificial
  Chemically synthesized peptide
TQFRLFNRSFTQALGKGGGKNLRIIRKGIHIIKKY

SEQ ID NO: 286 - protein, 35 aa, Artificial
  Chemically synthesized peptide
TFFRLFNRSFTQALDKGGGKNLRIIRKGIHIIKKY

SEQ ID NO: 287 - protein, 35 aa, Artificial
  Chemically synthesized peptide
TFFRLFNRSFTQALAKGGGKNLRIIRKGIHIIKKY

SEQ ID NO: 288 - protein, 35 aa, Artificial
  Chemically synthesized peptide
TFFRLFNRSFTQALGEGGGKNLRIIRKGIHIIKKY

SEQ ID NO: 289 - protein, 35 aa, Artificial
  Chemically synthesized peptide
TFFRLFSRSFTQALGKGGGKNLRIIRKGIHIIKKY

SEQ ID NO: 290 - protein, 35 aa, Artificial
  Chemically synthesized peptide
TFFRLFNRSFTQALGAGGGKNLRIIRKGIHIIKKY

SEQ ID NO: 291 - protein, 35 aa, Artificial
  Chemically synthesized peptide
TFFRLFDRSFTQALGKGGGKNLRIIRKGIHIIKKY

SEQ ID NO: 292 - protein, 35 aa, Artificial
  Chemically synthesized peptide
TFFRLFNRSFTQALGFGGGKNLRIIRKGIHIIKKY

SEQ ID NO: 293 - protein, 35 aa, Artificial
  Chemically synthesized peptide
TFFRAFARSFTQAAAKGGGKNLRIIRKGIHIIKKY

SEQ ID NO: 294 - protein, 35 aa, Artificial
  Chemically synthesized peptide
TFFRLFARSFTQAAGKGGGKNLRIIRKGIHIIKKY

SEQ ID NO: 295 - protein, 33 aa, Artificial
  Chemically synthesized peptide
TFFRLFNRSFTQLKGGGKNLRIIRKGIHIIKKY

SEQ ID NO: 296 - protein, 35 aa, Artificial
  Chemically synthesized peptide
TFFRLFNRSFTQALGSGGGKNLRIIRKGIHIIKKY

SEQ ID NO: 297 - protein, 35 aa, Artificial
  Chemically synthesized peptide
TLFRLFNRSFTQALGKGGGKNLRIIRKGIHIIKKY

SEQ ID NO: 298 - protein, 35 aa, Artificial
  Chemically synthesized peptide
TFFRLNFRSFTQALGKGGGKILRNIRKGIHIIKKY

SEQ ID NO: 299 - protein, 35 aa, Artificial
  Chemically synthesized peptide
TFFRLFNRSQTQALGKGGGKNLRIIRKGIHIIKKY

SEQ ID NO: 300 - protein, 35 aa, Artificial
  Chemically synthesized peptide
TFFRLFAAAFTQALGKGGGKNLRIIRKGIHIIKKY

SEQ ID NO: 301 - protein, 35 aa, Artificial
  Chemically synthesized peptide
TFFRLFNRSFTQALGKPYPKNLRIIRKGIHIIKKY

SEQ ID NO: 302 - protein, 35 aa, Artificial
  Chemically synthesized peptide
TFFRLFNRSAAAALGKGGGKNLRIIRKGIHIIKKY

SEQ ID NO: 303 - protein, 35 aa, Artificial
  Chemically synthesized peptide
TFFRLFFRSNTQALGKGGGKILRIIRKGIHINKKY

SEQ ID NO: 304 - protein, 35 aa, Artificial
  Chemically synthesized peptide
TFFRLFNRSFTQPLGKGGGKNLRIIRKGIHIIKKY

SEQ ID NO: 305 - protein, 35 aa, Artificial
  Chemically synthesized peptide
TAFRLANRSATQALGKGGGKNLRIIRKGIHIIKKY

SEQ ID NO: 306 - protein, 35 aa, Artificial
  Chemically synthesized peptide
TFFRLFNRSFTQAAAAGGGKNLRIIRKGIHIIKKY

SEQ ID NO: 307 - protein, 35 aa, Artificial
  Chemically synthesized peptide
TFFRLQNRSFTQALGKGGGKNLRIIRKGIHIIKKY

SEQ ID NO: 308 - protein, 35 aa, Artificial
  Chemically synthesized peptide
TFFRLFNRSFTQALPKGGGKNLRIIRKGIHIIKKY

SEQ ID NO: 309 - protein, 35 aa, Artificial
  Chemically synthesized peptide
TYYRLFNRSFTQALGKGGGKNLRIIRKGIHIIKKY

SEQ ID NO: 310 - protein, 34 aa, Artificial
  Chemically synthesized peptide
TFFRLFRSFTQALGKGGGKNLRIIRKGIHIIKKY